(19) United States
(12) Patent Application Publication
(10) Pub. No.: US 2022/0411477 A1
(43) Pub. Date: Dec. 29, 2022

(54) ANTI-MULLERIAN HORMONE POLYPEPTIDES

(71) Applicant: MONASH UNIVERSITY, Clayton, Victoria (AU)

(72) Inventors: Craig Anthony HARRISON, Clayton (AU); Kelly Louise WALTON, Clayton (AU)

(21) Appl. No.: 17/773,529

(22) PCT Filed: Oct. 30, 2020
(86) PCT No.: PCT/AU2020/051187

§ 371 Date: Apr. 29, 2022

(30) Foreign Application Priority Data
Oct. 30, 2019 (AU) 2019904097

(51) Int. Cl. C07K 14/575; C12N 15/861; A61P 35/00; A61P 15/18

(52) U.S. Cl. CPC C07K 14/575; C12N 15/861; A61P 35/00; A61P 15/18

(57) ABSTRACT

The present disclosure relates to anti-mullerian hormone (AMH) analogues, more particularly AMH analogues which are agonists of the AMH type II receptor (AMHR2). More particularly, the present disclosure relates to AMH analogues having a modification present within one or more of amino acid residues 533 to 548 of SEQ ID NO:1.

This application claims priority to AU2019904097 filed 30 Oct. 2019, the entire contents of which are herein incorporated by reference.

All documents cited or referenced herein, and all documents cited or referenced in herein cited documents, together with any manufacturer's instructions, descriptions, product specifications, and product sheets for any products mentioned herein or in any document incorporated by reference herein, are hereby incorporated herein by reference in their entirety.

The entire content of the electronic submission of the sequence listing is incorporated by reference in its entirety for all purposes.

### FIELD OF THE DISCLOSURE

The present disclosure relates to anti-mullerian hormone (AMH) analogues, more particularly AMH analogues which are agonists of the AMH type II receptor (AMHR2).

### BACKGROUND

Cancer is the second leading cause of death globally and is estimated to account for 9.6 million deaths in 2018 in the USA alone. In the USA, it is estimated that there will be just under 2 million young adult cancer survivors (aged 15-39) by 2026. Unfortunately, many of these cancer treatments can also cause infertility, sterility, or early menopause (Jeruss, J S et al., (2009) N Engl J Med 360, 902-911). Infertility or premature ovarian failure has been reported in 40% to 80% of cancer survivors due to chemotoxicity-induced accelerated loss of oocytes (Pereira N et al., (2017) J Oncol Pract 13, 643-651). The reproductive health of cancer survivors is a major concern for future quality of life of patients and will likely lead to psychological distress as infertility is a predictor of stress in present and future relationships (Rosen A et al., (2009) Semin Oncol Nurs 25, 268-277). Established fertility treatment options for female cancer patients are limited to invasive approaches that are only suitable for use in a subset of patients, while investigational approaches have limited utility (Woodruff T K et al., (2010), Nat Rev Clin Oncol 7, 466-475). Moreover, these invasive approaches come with logistical barriers as they are heavily dependent on timely patient referral and coordination of care between specialties which can limit patient access to the available options and prolong cancer treatment wait time. In addition, financial barriers due to high treatment costs and lack of coverage by certain insurance providers further restricts the applicability of invasive fertility treatments. The ideal oncofertility preservation treatment would be a non-interventional drug, administered alongside cancer therapy that preserves fertility in young women being treated for cancer.

The human AMH gene is located on chromosome 19 and its expression is sexually dimorphic. AMH is absolutely required for normal male reproductive tract development because it affects the regression of the Mullerian duct of the bipotential urogenital ridge, which if left undisturbed would give rise to the female reproductive tract structures such as the uterus, cervix, fallopian tubes and upper vagina (Cate et al., (1986) Cell 45:685-698). In males, expression of AMH (also known as mullerian inhibiting substance (MIS)) begins at 9 weeks gestation in the foetal testes and continues at high levels until puberty after which time expression levels fall dramatically. In females, AMH is produced only postnatally in granulosa cells from prepuberty through menopause at levels similar to adult males, after which expression ceases. In male foetuses, AMH causes regression of the Mullerian ducts, the precursors to the Fallopian tubes, uterus, cervix and upper vagina.

AMH exerts its biologic effect after binding to a heterodimer of type I and type II single transmembrane spanning serine threonine kinase receptors. AMH binds to the type II receptor which leads to cross-phosphorylation of the GS box kinase domain of the type I receptor by the type II receptor initiating signalling from the type I receptor. Subsequently SMAD 1, 5 and 8 are activated and together with SMAD 4 regulate gene transcription. AMH type II receptor (also referred to herein as AMHR2) is a 65-kDa protein and has been detected in embryonic and adult Mullerian structures, as well as in breast tissue, prostatic tissue, the gonads, motor neurons, and brain. Expression of AMHR2 can also be detected in the gonads, as well as in the ovarian coelomic epithelium.

There is a need in the art for compounds that facilitate preservation of fertility, for example by preventing premature ovarian failure caused by a cytotoxic drug or other drug treatment (e.g. chemotherapy) or to preserve ovarian reserve while undergoing a long-term treatment where pregnancy would be undesirable for example, during treatment for a chronic disease or disorder such as autoimmune disease.

### SUMMARY OF THE DISCLOSURE

Studies have shown that anti-mullerian hormone (AMH) is a measure of ovarian reserve (i.e. quality and quantity of primordial follicles (Visser J A et al., (2012) Nat Rev Endocrinol 8, 331-341). AMH assays can be used to clinically assess ovarian reserve during infertility treatment and after gonadotoxic cancer treatment or ovarian surgery (Victoria M et al., (2019) J Gynecol Obstet Hum Reprod 48, 19-24). Chemotherapeutic agents are postulated to damage the ovary by (i) inducing apoptosis in growing follicles and (ii) upregulating Akt-dependent primordial follicles recruitment. Together, these processes lead to rapid “burnout” of the ovarian reserve (R. R. Wong R R et al., (2014) Endocr Relat Cancer 21, R227-233).

The inventors used site-directed mutagenesis to identify the putative type II receptor (AMHR2) binding site on human AMH. Utilising cell models, they identified AMH analogues that increase signalling from the AMH receptor complex relative to wild-type AMH mature domain. It is thought that the AMH analogues may be useful as agonists of the AMH type II receptor. These analogues may have applications in oncofertility (i.e. preservation of fertility during and after cancer treatment) as well as in treatment of gynecological cancers and as a reversible contraceptive agent. Surprisingly, the inventors found that mutation of Gly533 in the wild-type AMH mature domain sequence resulted in significantly increased AMH activity compared to wild-type/native AMH (SEQ ID NO:1). This finding was surprising given that glycine is not a residue typically involved in protein-protein interactions. It is anticipated that such analogues will have utility in oncologic and fertility-related applications as a fertility preservation agent during chemotherapy, as an agent for the treatment of genealogical cancer and as a potential reversible contraceptive that can limit damage to the ovarian reserve caused by gonadotrophic agents.

In one aspect, the disclosure provides an anti-mullerian hormone (AMH) analogue, comprising a polypeptide sequence which has at least 80% identity to a native AMH polypeptide set forth in SEQ ID NO:1 or at least 80% identity to amino acid residues 452 to 560 of SEQ ID NO:1 (FIG. 1A). Unless indicated otherwise, residue numbering throughout the specification is with reference to the human AMH precursor shown in SEQ ID NO:1 (FIG. 1A), where the first residue in the precursor sequence (e.g. methionine) is numbered as position 1 and the last residue in the sequence is numbered as position 560.

In one example, the AMH analogue comprises a sequence having at least 85%, 90%, 92%, 94%, 95%, 96%, 97%, 98%, or 99% identity to a native AMH polypeptide set forth in SEQ ID NO:1 or at least 80% identity to amino acid residues 452 to 560 of SEQ ID NO:1 (FIG. 1A).

In another aspect, the disclosure provides an AMH analogue comprising a polypeptide sequence comprising at least one amino acid residue modification relative to a native human AMH polypeptide set forth in SEQ ID NO:1, wherein the modification is present within amino acid residues 452 to 560 of SEQ ID NO:1.

In another aspect, the disclosure provides an isolated anti-mullerian hormone (AMH) analogue, comprising a C-terminal domain comprising an amino acid sequence which has at least 80% identity to amino acid residues 452 to 560 of SEQ ID NO:1 (FIG. 1A).

In another aspect, the disclosure provides an isolated anti-mullerian hormone (AMH) analogue, comprising a C-terminal domain sequence, wherein the C-terminal domain comprises at least one amino acid residue modification relative to a native human mature processed AMH polypeptide set forth in SEQ ID NO:5, wherein the modification is present within one or more of amino acid residues 533 to 548 of SEQ ID NO:1. In some examples, the modification present within amino acid residues 533 to 548 of SEQ ID NO:1 is at least one amino acid substitution. In some examples, the modification present within amino acid residues 533 to 548 of SEQ ID NO:1 is a single amino acid substitution.

In one example, the C-terminal domain comprises a sequence having at least 85%, 90%, 92%, 94%, 95%, 96%, 97%, 98%, or 99% identity to amino acid residues 452 to 560 of SEQ ID NO:1 (FIG. 1A).

In one example, the AMH analogue further comprises an N-terminal domain comprising an amino acid sequence which has at least 80% identity to amino acid residues 30 to 447 of SEQ ID NO:1. In one example, the N-terminal domain comprises a sequence having at least 85%, 90%, 92%, 94%, 95%, 96%, 97%, 98%, or 99% identity to amino acid residues 30 to 447 of SEQ ID NO:1 (FIG. 1A). In some examples, the AMH analogue further comprises a N-terminal domain comprising a sequence which has at least 90% identity to amino acid residues 30 to 451 of SEQ ID NO:1. In some examples, the AMH analogue further comprises a N-terminal domain comprising a sequence which has at least 90% identity to amino acid residues 26 to 447 of SEQ ID NO:1. In some examples, the AMH analogue further comprises a N-terminal domain comprising a sequence which has at least 90% identity to amino acid residues 26 to 451 of SEQ ID NO:1.

In one example, the N-terminal domain and C-terminal domain are not covalently bound. In one example, the N-terminal domain and C-terminal domain are not covalently bound by a peptide bond, i.e. the N-terminal domain and C-terminal domain are separate polypeptides. In an alternative example, the N-terminal domain and C-terminal domain are covalently bound. In one example, the covalent bond comprises a peptide bond.

In some examples, the N-terminal domain comprises a proprotein convertase site that comprises X₁X₂X₃RKKRX₈X₉X₁₀X₁₁ (SEQ ID NO:37), wherein X₁ is absent or isoleucine, X₂ is absent or serine, X₃ is absent or serine, X₈ is absent or serine, X₉ is absent or valine, X₁₀ is absent or serine and X₁₁ is absent or serine. In one example, the proprotein convertase site comprises ISSRKKRSVSS (SEQ ID NO:6). In one example, the proprotein convertase site comprises RKKR (SEQ ID NO:40).

In some examples the activity of the analogue is comparable to, or greater than the activity of native human AMH. In a particular example, the native human AMH comprises the polypeptide sequence of residues 452 to 560 of SEQ ID NO:1 or the polypeptide sequence set forth in SEQ ID NO:5.

In one example, the activity of the AMH analogue is equivalent to native human AMH, about 2-fold greater, about 2.5-fold greater, about 3-fold greater, about 3.5-fold greater, about 4-fold greater, about 4.5-fold greater, about 5-fold greater or greater than 5-fold compared to the activity of the native mature processed AMH polypeptide. In some embodiments, the native mature processed AMH polypeptide comprises the sequence set forth in SEQ ID NO:5. In some embodiments, the native mature processed AMH polypeptide comprises the sequence set forth in SEQ ID NO:36. In one example, the activity of the analogue is determined by luciferase assay.

In one example, the activity of the AMH analogue is at least 2.5-fold greater, or between 2.5-fold and 5-fold greater.

In one example, the sequence comprises a single amino acid residue modification.

In one example, the sequence comprises two amino acid residue modifications.

In one example, the sequence comprises three amino acid residue modification.

In another example, the sequence comprises no more than 5 amino acid residue modifications.

In one example, the modification is a substitution.

In one example, the C-terminal domain comprises at least one amino acid residue modification relative to a native human AMH polypeptide set forth in SEQ ID NO:5, wherein the modification is present within amino acid residues 533 to 548 of SEQ ID NO:1.

In another example, the C-terminal domain comprises a single amino acid residue modification relative to the mature processed AMH polypeptide comprising the sequence set forth in SEQ ID NO:5. In another example, the C-terminal domain comprises two amino residue modifications relative to the mature processed AMH polypeptide comprising the sequence set forth in SEQ ID NO:5. In another example, the C-terminal domain comprises three amino residue modifications relative to the mature processed AMH polypeptide comprising the sequence set forth in SEQ ID NO:5.

In some examples, the C-terminal domain optionally comprises one or more further amino acid residues at the N-terminus (for example, relative to a native human AMH polypeptide set forth in SEQ ID NO:5).

In some examples, the modification is an amino acid substitution.

In one example, the amino acid residue substitution is located at residue 533, 535 and/or 548 of SEQ ID NO:1.

In one example, the amino acid substitution is selected from the group consisting of G533, L535, and G533+L535. In another example, the substitution is selected from the group consisting of L535M, and G533A+L535M.

In one example, the amino acid modification is at G533 of SEQ ID NO:1. In another example, the modification is selected from the group consisting of G533A, G533S, G533K, G533L and G533R of SEQ ID NO:1. In another example, the modification is selected from the group consisting of G533A, G533S and G533K of SEQ ID NO:1.

In a particular example, the modification is G533K of SEQ ID NO:1.

In one example, the one or more further amino acid residues at the N-terminus comprise SVSS (SEQ ID NO:41).

In yet another example, the AMH analogue is an agonist of the AMH type II receptor (AMHR2). In one example, the AMH analogue specifically binds to AMHR2.

In some examples, the receptor binding affinity of the AMH analogue to AMHR2 relative to native human AMH is increased by at least 2-fold, at least 3-fold, at least 4-fold, at least 5-fold, or greater than 5-fold.

In one example, the AMH analogue is a human polypeptide.

In one example, the AMH is derived from a AMH precursor comprising the sequence set forth in SEQ ID NO:3. In one example, the amino acid residue at G533 (i.e. residue 535 in SEQ ID NO; 3) of the precursor is modified by substitution to alanine, serine or lysine.

In some examples, the AMH analogue or AMH precursor sequence is further modified to improve proteolytic processing. In one example the native proteolytic processing site at R448 to R451 is replaced with the sequence set forth in ISSRKKRSVSS (SEQ ID NO:6; FIG. 1B).

In some examples, the AMH analogue or AMH precursor is further modified to enhance recombinant production by replacing the native signalling peptide. In one example, the native human AMH signalling sequence consisting of the sequence set forth in MRDLPLTSLALVLSALGALLGTEAL (SEQ ID NO:7) is replaced with a rat albumin signal peptide sequence. In one example, the rat albumin signal sequence comprises or consists of the sequence set forth in MKWVTFLLLLFISGSAFS (SEQ ID NO:8).

In one example, the AMH analogue or AMH precursor comprises modifications to both the proteolytic processing site and signal peptide sequence.

In some examples, the AMH analogue or AMH precursor may further comprise a His tag, for example His6 tag to assist purification. In some examples, the N-terminal domain may further comprise an N-terminal His tag.

In a particular example, the AMH precursor comprises or consists of the sequence set forth in:

(SEQ ID NO: 3)
MKWVTFLLLLFISGSAFSHHHHHHPAVGTSGLIFREDLDWPPGSPQEPLC
LVALGGDSNGSSSPLRVVGALSAYEQAFLGAVQRARWGPRDLATFGVCNT
GDRQAALPSLRRLGAWLQDPGGQRLVVLHLEEVTWEPTPSLRFQEPPPGG
AGPPELALLVLYPGPGPEVTVTRAGLPGAQSLCPSRDTRYLVLAVDRPAG
AWRGSGLALTLQPRGEDSRLSTARLQALLFGDDHRCFTRMTPALLLLPRS
EPAPLPAHGQLDTVPFPPPRPSAELEESPPSADPFLETLTRLVRALRVPP
ARASAPRLALDPDALAGFPQGLVNLSDPAALERLLDGEEPLLLLLRPTAA
TTGDPAPLHDPTSAPWATALARRVAAELQAAAAELRSLPGLPPATAPLLA
RLLALCPGGPGGLGDPLRALLLLKALQGLRVEWRGRDPRGPGISSRKKRS
VSSSAGATAADGPCALRELSVDLRAERSVLIPETYQANNCQGVCGWPQSD
RNPRYGNHVVLLLKMQARGAALARPPCCVPTAYAGKLLISLSEERISAHH
VPNMVATECGCR

wherein the amino acid residue at G533 (shown as bold and underlined and numbered by reference to the native sequence provided in SEQ ID NO:1) is modified by amino acid substitution.

In another example, the AMH analogue comprises or consists of SEQ ID NO:3 lacking the signal sequence set forth as MKWVTFLLLLFISGSAFS (SEQ ID NO:8) wherein the amino acid residue at G533 (shown as bold and underlined) is modified by amino acid substitution.

For the avoidance of doubt, reference to G533 refers to the Gly located at residue position 533 in the native human AMH sequence shown in SEQ ID NO:1. G533 corresponds to G535 in the modified sequence set forth in SEQ ID NO:3.

In one example, the amino acid substitution at G533 of SEQ ID NO:3 is G533A, G533S or G533K.

In another aspect, the disclosure provides an AMH analogue having a C-terminal domain comprising a sequence selected from the group consisting of:

(SEQ ID NO: 9)
(i)SAGATAADGPCALRELSVDLRAERSVLIPETYQANNCQGVCGWPQSD
RNPRYGNHVLLLKMQARGAALARPPCCVPTAYAKKLLISLSEERISAHHV
PNMVATECGCR;
(SEQ ID NO: 10)
(ii)SAGATAADGPCALRELSVDLRAERSVLIPETYQANNCQGVCGWPQS
DRNPRYGNHVVLLLKMQARGAALARPPCCVPTAYASKLLISLSEERISAH
HVPNMVATECGCR;
(SEQ ID NO: 11)
(iii)SAGATAADGPCALRELSVDLRAERSVLIPETYQANNCQGVCGWPQ
SDRNPRYGNHVVLLLKMQARGAALARPPCCVPTAYAAKLLISLSEERISA
HHVPNMVATECGCR;
(SEQ ID NO: 12)
(iv)SAGATAADGPCALRELSVDLRAERSVLIPETYQANNCQGVCGWPQS
DRNPRYGNHVVLLLKMQARGAALARPPCCVPTAYAHKLLISLSEERISAH
HVPNMVATECGCR;
(SEQ ID: 13)
(v)SAGATAADGPCALRELSVDLRAERSVLIPETYQANNCQGVCGWPQSD
RNPRYGNHVVLLLKMQARGAALARPPCCVPTAYAGKMLISLSEERISAHH
VPNMVATECGCR;
(SEQ ID NO: 14)
(vi)SAGATAADGPCALRELSVDLRAERSVLIPETYQANNCQGVCGWPQS
DRNPRYGNHVVLLLKMQARGAALARPPCCVPTAYAAKMLISLSEERISAH
HVPNMVATECGCR;
and
(SEQ ID NO: 15)
(vii)SAGATAADGPCALRELSVDLRAERSVLIPETYQANNCQGVCGWPQ
SDRNPRYGNHVVLLLKMQARGAALARPPCCVPTAYAAKMLISLSEERISA
HKVPNMVATECGCR.

In some examples, the AMH analogue further comprises a fusion partner selected from one or more of an Fc protein, a detection tag, a purification tag, or a carrier molecule.

In a further aspect, the disclosure provides a vector comprising a polynucleotide encoding the AMH analogue or AMH precursor described herein operably linked to a promoter. In one example, the polynucleotide comprises or consists of the sequence set forth in:

(SEQ ID NO: 4)
atgaagtgggtaacctttctcctcctcctcttcatctccggttctgcctt
ttcccatcatcatcatcatcatccagctgtgggcaccagtggcctcatct
tccgagaagacttggactggcctccaggcagcccacaagagcctctgtgc
ctggtggcactgggcggggacagcaatggcagcagctcccccctgcgggt
ggtgggggctctaagcgcctatgagcaggccttcctgggggctgtgcaga
gggcccgctggggcccccgagacctggccaccttcggggtctgcaacacc
ggtgacaggcaggctgccttgccctctctacggcggctgggggcctggct
gcaggaccctggggggcagcgcctggtggtcctacacctggaggaagtga
cctgggagccaacaccctcgctgaggttccaggagcccccgcctggagga
gctggccccccagagctggcgctgctggtgctgtaccctgggcctggccc
tgaggtcactgtgacgagggctgggctgccaggtgcccagagcctctgcc
cctcccgagacacccgctacctggtgttagcggtggaccgccctgcgggg
gcctggcgcggctccgggctggccttgaccctgcagccccgcggagagga
ctcccggctgagtaccgcccggctgcaggcactgctgttcggcgacgacc
accgctgcttcacacggatgaccccggccctgctcctgctgccgcggtcc
gagcccgcgccgctgcctgcgcacggccagctggacaccgtgcccttccc
gccgcccaggccatccgcggaactggaggagtcgccacccagcgcagacc
ccttcctggagacgctcacgcgcctggtgcgggcgctgcgggtccccccg
gcccgggcctccgcgccgcgcctggccctggatccggacgcgctggccgg
cttcccgcagggcctagtcaacctgtcggaccccgcggcgctggagcgcc
tactcgacggcgaggagccgctgctgctgctgctgaggcccactgcggcc
accaccggggatcctgcgcccctgcacgaccccacgtcggcgccgtgggc
cacggccctggcgcgccgcgtggctgctgaactgcaagcggcggctgccg
agctgcgaagcctcccgggtctgcctccggccacagccccgctgctggcg
cgcctgctcgcgctctgtccaggaggccccggcggcctcggcgatcccct
gcgagcgctgctgctcctgaaggcgctgcagggcctgcgcgtggagtggc
gcgggcgggatccgcgcgggccgggtatctcatcgagaaagaaacgctca
gtctcatcaagcgcgggggccaccgccgccgacgggccgtgcgcgctgcg
cgagctcagcgtagacctccgcgccgagcgctccgtactcatccccgaga
cctaccaggccaacaattgccagggcgtgtgcggctggcctcagtccgac
cgcaacccgcgctacggcaaccacgtggtgctgctgctgaagatgcaggc
ccgtggggccgccctggcgcgcccaccctgctgcgtgcccaccgcctacg
cgggcaagctgctcatcagcctgtcggaggagcgcatcagcgcgcaccac
gtgcccaacatggtggccaccgagtgtggctgccggtaa

wherein the polynucleotide sequence at nucleotides 1603 to 1605 (shown as bold and underlined) is modified to encode an alanine (A), serine (S) or lysine (K).

In one example, polynucleotide residues 1603 to 1605 in SEQ ID NO:4 is selected from the group consisting of gct, gcc, gca, gcg, tct, tcc, tca, tcg, aaa or aag.

In a further aspect, there is provided an AMH precursor comprising a polypeptide comprising a C-terminal domain sequence, wherein the C-terminal domain comprises at least one amino acid residue modification relative to a native human mature processed AMH polypeptide set forth in SEQ ID NO:5, wherein the modification is present within one or more of amino acid residues 533 to 548 of SEQ ID NO:1.

In some examples, the modification is G533A, G533K or G533S.

In some examples, the polypeptide comprises the sequence set forth in SEQ ID NO:1 or SEQ ID NO:3, wherein the amino acid corresponding to G533 of SEQ ID NO:1 is modified by substitution to G533A, G533S or G533K.

In a further aspect, there is provided an AMH polynucleotide comprising the sequence set forth in SEQ ID NO:4, wherein the polynucleotide sequence at nucleotides 1603 to 1605 of SEQ ID NO:4 is modified to encode an alanine (A), serine (S) or lysine (K).

In a further aspect, there is provided an AMH polynucleotide comprising the sequence set forth in SEQ ID NO:34, wherein the polynucleotide sequence at nucleotides 1597 to 1599 of SEQ ID NO:33 is modified to encode an alanine (A), serine (S) or lysine (K).

In a further aspect, there is provided an AMH polynucleotide comprising the sequence set forth in SEQ ID NO:35, wherein the polynucleotide sequence at nucleotides 244 to 246 is modified to encode an alanine (A), serine (S) or lysine (K).

In one example, the AMH analogue is recombinantly produced. In one example, the AMH precursor is recombinantly produced.

In one example, the polypeptide encoding the AMH analogue is expressed in a vector, preferably, a mammalian vector. In another example, the vector is a bacterial vector. In a further example, the vector is a viral vector. In a particular example, the vector is the pcDNA3.1 vector.

In some examples, the vector is an adeno-associated virus (AAV) vector. In a particular example, the vector is AAV serotype 9 (AAV-9).

In a further aspect, the present disclosure provides a host cell comprising the vector described herein. In one example, the host cell is a mammalian host cell. In another example, the host cells are selected from embryonic kidney, CHO, or ovarian cells. In one example, the host cells are HEK293T cells.

In a further aspect, the present disclosure provides a composition comprising the AMH analogue described herein or the vector encoding the AMH analogue or AMH precursor described herein.

In one example, the composition comprises an AMH analogue comprising a sequence having at least 80% identity to amino acid residues 452 to 560 of SEQ ID NO:1. In some examples, the AMH analogue further comprises a N-terminal domain comprising a sequence which has at least 90% identity to amino acid residues 30 to 447 of SEQ ID NO:1. In some examples, the AMH analogue further comprises a N-terminal domain comprising a sequence which has at least 90% identity to amino acid residues 26 to 447 of SEQ ID NO:1. In some examples, the AMH analogue further comprises a N-terminal domain comprising a sequence which has at least 90% identity to amino acid residues 30 to 451 of SEQ ID NO:1. In some examples, the AMH analogue further comprises a N-terminal domain comprising a sequence which has at least 90% identity to amino acid residues 26 to 451 of SEQ ID NO:1. In some examples, the C-terminal domain and N-terminal domain are not covalently bound. In some examples, the AMH analogue comprises a quaternary complex comprising two C-terminal domains and two N-terminal domains. In some examples, the quaternary complex comprises a C-terminal homodimer and an N-terminal homodimer.

In another example, the N-terminal domain comprises a sequence having at least 92%, 94%, 95%, 96%, 97%, 98%, 99% or 100% identity to amino acid residues 30 to 447 of SEQ ID NO:1. In another example, the N-terminal domain comprises a sequence having at least 92%, 94%, 95%, 96%, 97%, 98%, 99% or 100% identity to amino acid residues 30 to 451 of SEQ ID NO:1. In another example, the N-terminal domain comprises a sequence having at least 92%, 94%, 95%, 96%, 97%, 98%, 99% or 100% identity to amino acid residues 26 to 447 of SEQ ID NO:1. In another example, the N-terminal domain comprises a sequence having at least 92%, 94%, 95%, 96%, 97%, 98%, 99% or 100% identity to amino acid residues 26 to 451 of SEQ ID NO:1.

In one example, the composition comprises an AMH analogue comprising a C-terminal domain selected from the group consisting of SEQ ID NO:9, SEQ ID NO:10, SEQ ID NO:11, SEQ ID NO:12, SEQ ID NO:13, SEQ ID NO:14 or SEQ ID NO:15.

Preferably, the AMH analogue according to any aspect when administered to a subject is a quaternary complex comprising two N-terminal domains and two C-terminal domains. In one example, the AMH analogue according to any aspect when administered to a subject is a quaternary complex comprising an N-terminal homodimer and a C-terminal homodimer. Preferably, the AMH analogue or AMH precursor following expression from the vector is a homodimer. In certain examples, the monomers are linked by disulphide bonds. In certain examples, the N-terminal domains are linked by disulphide bonds. In certain examples, the C-terminal domains are linked by disulphide bonds. In other examples, the monomers are joined by a linker. In another example, the monomers are non-covalently associated.

In one example, the AMH analogue is provided in a therapeutically effective amount. A “therapeutically effective amount” may differ depending on the intended use. In one example, a therapeutically effective amount is an amount administered at a concentration to provide complete arrest of folliculogenesis in the subject. In another example, a therapeutically effective amount is considered to be an amount which is sufficient to increase the concentration of AMH analogue in the blood of the subject by 10-50% higher, or by 50 to 100% higher compared to the absence of AMH. In one example, a therapeutically effective amount is an amount which is sufficient to increase the concentration of the AMH analogue in the blood of the subject between 1 μg/ml and 5 μg/ml.

In another example, the composition comprises a pharmaceutically acceptable carrier.

In one example, the composition is administered to a human or non-human primate. In another example, the composition is administered to a non-human animal. In one example, the composition is administered to a non-human animal selected from cat, dog and horse. In one example, the subject has cancer. In one example, the cancer is a gynaecological cancer. In another example, the subject is undergoing a treatment for cancer such as immunotherapy, cell therapy, radiotherapy or chemotherapy. In another example, the subject is mentally incapacitated such that sterilisation of the subject, and/or suppression of folliculogenesis is in the best interest of the welfare of the subject.

Administration of the composition may be by any method and route known to those skilled in the art. Administration may be intraperitoneal or subcutaneous or injection directly into the follicle. Administration may be transdermal. In some examples, the AMH analogue is administered for consistent delivery, for example as a one-time injection in vector format for gene therapy where permanent contraception is desirable. In other examples, the AMH analogue is delivered in intermittent pulse format wherein a single administration is followed by an interval of no administration, for example where it is desirable to have temporary arrest of folliculogenesis such as in a temporary method of contraception or where pregnancy is desired at a later period in the subject's lifetime. In some examples, pulsed administration comprises administration of the AMH analogue followed by an interval of at least 3 days, at least 7 days, between about 7 days and 3 weeks of no treatment between pulsed administration of the composition disclosed herein.

The composition of the disclosure may be provided in the form of a transdermal patch, vaginal ring, biogel or as a coating onto an implantable contraceptive device such as an intra uterine device (IUD).

The composition may be administered alone or in combination with a cell therapeutic, a immunotherapeutic, chemotherapeutic or radiotherapeutic agent.

In another aspect, the disclosure provides a method of preventing a decline in the functional ovarian reserve in a female subject, comprising administering to the subject, an AMH analogue or a composition of the disclosure. In some examples, preventing a decline in the functional ovarian reserve relates to a method of preserving ovarian follicle reserve in a female subject. In some examples, preventing a decline in the functional ovarian reserve relates to reducing the number of primordial follicles being recruited by at least 10% compared to in the absence of the AMH analogue, or reducing the number of primordial follicles being recruited by between 10% and 99% or causing a complete arrest in folliculogenesis, or slowing down of primordial follicle activation, as compared to in the absence of the AMH analogue.

In another aspect, the disclosure provides a method of contraception in a female subject, comprising administering to the subject an AMH analogue or a composition of the disclosure. In one example, the subject is a pre-menopausal female subject. In one example, the subject or a pre-pubescent female subject. Use of the AMH analogues of the disclosure may be short term or long term.

In another aspect, the disclosure provides a method for ovarian and/or uterine protection in a subject, comprising administering to the subject an AMH analogue or composition of the disclosure.

In one example, the subject is a human subject over the age of 35.

In one example, the subject is undergoing or will undergo treatment for cancer. In one example, the subject is undergoing immunotherapy, cell therapy, chemotherapy or radiotherapy treatment for cancer. In one example, the subject is undergoing treatment for a chronic disease or disorder.

In some examples, the method inhibits the natural age-related decline in functional ovarian reserve by at least 10%, or at least 20%, or at least 30% or at least 40%, or at least 50% or more than 50% as compared to an age-matched subject not administered the AMH analogue described herein.

In some examples, the subject is undergoing or about to undergo treatment for cancer or is undergoing treatment or about to undergo treatment for a chronic disease or disorder.

In some examples, the subject has an autoimmune disease and will be treated with, or is currently being treated with, or has been treated with, an immunotherapy.

In some examples, the subject will be treated with, or is currently being treated with, or has been treated with a cytotoxic drug or cytotoxic agent that causes cell death or cell damage to cells in the uterus or ovary.

In another aspect, the disclosure provides a method for treating a gynaecological cancer in a subject, comprising administering to the subject an AMH analogue or composition of the disclosure.

In another aspect, the disclosure provides an AMH analogue as described herein in the manufacture of a medicament for preserving ovarian follicle reserve, contraception, uterine protection, or treating a gynaecological cancer.

In some examples the female subject is in need of preserving their ovarian reserve, or has a need or desire to delay reproduction, or wherein the subject has, or is pre-disposed to any one of the following: diminished ovarian reserve (DOR), premature ovarian ageing (POA), primary ovarian insufficiency (POI), endometriosis, BRAC1 mutations, Turner syndrome, an autoimmune disease, thyroid autoimmunity, adrenal autoimmunity or autoimmunity polyglandular syndromes.

In a particular example according to any method or use described herein, the AMH analogue is administered as a quaternary complex comprising an N-terminal homodimer and a C-terminal homodimer.

In another aspect, the disclosure provides a kit for use according to any method described herein, the kit comprising:

(i) an administration device comprising the AMH analogue described herein; and

(ii) instructions for use in a subject.

In one example, the administration device is selected from a pump or infusion device, one or more single dose, or multi-dose pre-loaded injection syringes or a transdermal patch. In one example, the pump is an osmotic pump e.g. an alzet pump. In one example, the administration device is an autoinjector as described in for example, U.S. Pat. Nos. 5,267,963, 6,277,097, 6,386,306, or 6,793,646.

### BRIEF DESCRIPTION OF THE FIGURES

FIG. 1 (A) Annotated amino acid sequence of wild-type human AMH protein sequence (GenBank AAH49194; SEQ ID NO:1). The signal peptide is underlined and corresponds to amino acids 1-25. The proprotein convertase recognition site (also referred to as “proprotein convertase site”) includes Arg448 to Arg451 and is indicated by shading with cleavage occurring between the Arg451 and Ser452. The prodomain includes the sequence from Arg26 to Arg451. The mature domain sequence (Ser452 to Arg560; SEQ ID NO:5) is indicated in bold. Gly533 is indicated as bold and underline in the mature domain sequence. (B) Annotated amino acid sequence of modified human AMH (hAMH+SCUT+RSA; SEQ ID NO:3). The signal peptide is underlined and corresponds to amino acids 1-18. The hexa-histidine tag is indicated by bold and underline. The super-cut (SCUT) proprotein convertase recognition sequence (Ile447a to Ser451d) is shaded grey with cleavage occurring between Arg451 and Ser451a. The prodomain is the sequence from Pro30 to Arg451. Processed AMH comprises amino acids Ser451a to Arg560. The mature domain is indicated in bold (Ser452 to Arg560).

FIG. 2 shows expression of mAMH variants. Modifications were made to the mAMH cDNA via in vitro site-directed mutagenesis. To assess whether the modifications affected precursor processing, conditioned media from transfected HEK-293T cells was concentrated 12.5-fold and analysed by Western blotting under reducing conditions. The blots were probed with mAb-5/6A which specifically recognises a region toward the C-terminus of AMH. The 12.5 kDa monomeric mature domain and 70 kDa monomeric AMH proprotein are shown.

FIG. 3 shows expression of a first cohort of hAMH mutants. Modifications were made to the hAMH+SCUT+RSA cDNA via in vitro site-directed mutagenesis. To assess whether the mutations prevented protein secretion, conditioned media from transfected HEK-293T cells was concentrated 12.5-fold and analysed by Western blotting under reducing conditions. The blots were probed with mAb-5/6A which specifically recognises a region towards the C-terminus of AMH. The 12.5 kDa monomeric mature domain is shown.

FIG. 4 shows expression of a second cohort of hAMH mutants. Modifications were made to the hAMH+SCUT+RSA cDNA via in vitro site-directed mutagenesis. To assess whether the mutations prevented protein secretion, conditioned media from transfected HEK-293T cells was concentrated 12.5-fold and analysed by Western blotting under reducing conditions. The blots were probed with mAb-5/6A (Lower panel) which specifically recognises a region toward the C-terminus of AMH, and mAb-9/6A (Upper panel) which specifically detects the processed hAMH prodomain. The 12.5 kDa monomeric mature domain (Lower panel) and 55 kDa processed AMH prodomain (Upper panel) are shown.

FIG. 5 shows expression of third cohort of hAMH mutants. Modifications were made to the hAMH+SCUT+RSA cDNA via in vitro site-directed mutagenesis. To assess whether the mutations prevented protein secretion, conditioned media from transfected HEK-293T cells was concentrated 12.5-fold and analysed by Western blotting under reducing conditions. The blots were probed with mAb-5/6A which specifically recognises a region toward the C-terminus of AMH. The 12.5 kDa monomeric mature domain is shown.

FIG. 6 shows expression of fourth cohort of hAMH mutants. Modifications were made to the hAMH+SCUT+RSA cDNA via in vitro site-directed mutagenesis. To assess whether the mutations prevented protein secretion, conditioned media from transfected HEK-293T cells was concentrated 12.5-fold and analysed by Western blotting under reducing conditions. The blots were probed with mAb-5/6A (Lower panel) which specifically recognises a region toward the C-terminus of AMH, and mAb-9/6A (Upper panel) which specifically detects the processed hAMH prodomain. The 12.5 kDa monomeric mature domain (Lower panel) and 55 kDa processed AMH prodomain (Upper panel) are shown.

FIG. 7 shows Co-IMAC purification of hAMH variants. 200 mL of media conditioned by transiently transfected cells was concentrated to ˜1 mL and made back to a final volume of 5 mL with binding buffer. The concentrated conditioned media was incubated in a column containing HisPur™ cobalt resin for ˜2.5 hours. Unbound proteins were collected and the column then washed twice with PBS. To elute bound proteins, the HisPur™ cobalt resin was incubated in 3 mL of PBS containing 500 mM imidazole for ˜2.5 hours. To recover any proteins remaining bound, the resin was incubated in 3 mL of PBS containing 1M imidazole for ˜1 hour. 10 μL of each fraction was separated by SDS-PAGE followed by Western transfer. Recovery was assessed by probing the blot with mAb-5/6A.

FIG. 8 shows activity of G533A, G533S and G533K mutants. COV434 human granulosa cells, transfected with a Smad1/5-responsive luciferase reporter and AMHR2, were treated overnight with increasing concentrations (A, 3.1-50 ng/mL; B, 0.62-50 ng/mL) of Co-IMAC purified hAMH variants. The measured luciferase activity is presented as the fold-change relative to an adjusted value of 1.0 for the mean of control wells.

FIG. 9 shows activity of G533H, H548K, L535M and G533A+L535M mutants. COV434 human granulosa cells, transfected with a Smad1/5-responsive luciferase reporter and AMHR2, were treated overnight with increasing concentrations (0.62-50 ng/mL) of Co-IMAC purified hAMH variants. The measured luciferase activity is presented as the fold-change relative to an adjusted value of 1.0 for the mean of control wells.

FIG. 10 shows processing of AMH to form the mature hormone (A) and a structural model of mature AMH with the wrist and finger domains labelled (B).

FIG. 11 shows a sequence alignment of processed mature AMH from human, cat, dog and horse. Alignment prepared using ClustalW (Larkin et al., (2007). Bioinformatics, 23, 2947-2948; Thompson et al., (1994). Nucleic Acids Res., 22, 4673-4680). Figure prepared using ESPript (Robert & Gouet (2014) Nucleic Acids Res., 42(W1), W320-W324).

FIG. 12 shows activity of mature processed AMH produced from hAMH+SCUT+RSA compared to activity of hAMH purchased from R&D Systems. The activity was determined using the luciferase assay described herein.

Key to Sequence Listing

SEQ ID NO:1 is the amino acid sequence of native human AMH precursor polypeptide

SEQ ID NO:2 is the amino acid sequence of native mouse AMH precursor polypeptide

SEQ ID NO:3 is the amino acid sequence of hAMH+SCUT+RSA (human AMH precursor with modified signal sequence, hexahistindine purification tag and modified proteolytic site)

SEQ ID NO:4 is the nucleotide sequence of hAMH+SCUT+RSA (human AMH precursor with modified signal sequence, hexahistindine purification tag and modified proteolytic site)

SEQ ID NO:5 is the sequence of human mature processed AMH polypeptide

SEQ ID NO:6 is the sequence of the proteolytic processing site used in hAMH+SCUT+RSA

SEQ ID NO:7 is the sequence of the human AMH leader/signal sequence

SEQ ID NO:8 is the sequence of the leader/signal sequence used in hAMH+SCUT+RSA

SEQ ID NO:9 is the sequence of an AMH analogue

SEQ ID NO:10 is the sequence of an AMH analogue

SEQ ID NO:11 is the sequence of an AMH analogue

SEQ ID NO:12 is the sequence of an AMH analogue

SEQ ID NO:14 is the sequence of an AMH analogue

SEQ ID NO:14 is the sequence of an AMH analogue

SEQ ID NO:15 is the sequence of an AMH analogue

SEQ ID NO:16 is a signal sequence

SEQ ID NO:17 is a signal sequence

SEQ ID NO:18 is a signal sequence

SEQ ID NO:19 is a signal sequence

SEQ ID NO:20 is a signal sequence

SEQ ID NO:21 is a signal sequence

SEQ ID NO:23 is a signal sequence

SEQ ID NO:24 is a signal sequence

SEQ ID NO:25 is a signal sequence

SEQ ID NO: 26 is a signal sequence

SEQ ID NO:27 is the nucleotide sequence of mouse AMH precursor polynucleotide

SEQ ID NO:28 is the nucleotide sequence of horse AMH precursor polynucleotide

SEQ ID NO:29 is the nucleotide sequence of dog AMH precursor polynucleotide

SEQ ID NO:30 is the nucleotide sequence of cat AMH precursor polynucleotide

SEQ ID NO:31 is the amino acid sequence of horse AMH precursor polypeptide

SEQ ID NO:32 is the amino acid sequence of dog AMH precursor polypeptide

SEQ ID NO:33 is the amino acid sequence of cat AMH precursor polypeptide

SEQ ID NO:34 is the nucleotide sequence of human AMH precursor polynucleotide

SEQ ID NO:35 is the nucleotide sequence encoding human mature processed AMH

SEQ ID NO:36 is the amino acid sequence of processed hAMH+SCUT+RSA

SEQ ID NO:37 is the sequence of a proteolytic processing site

SEQ ID NO:38 is the sequence of a proteolytic processing site

SEQ ID NO:39 is the sequence of a proteolytic processing site

SEQ ID NO:40 is the sequence of a proteolytic processing site

SEQ ID NO:41 is the N-terminal extension to the C-terminal domain

### DETAILED DESCRIPTION

### General Techniques and Selected Definitions

The term “and/or”, e.g., “X and/or Y” shall be understood to mean either “X and Y” or “X or Y” and shall be taken to provide explicit support for both meanings or for either meaning.

As used herein, the terms “a”, “an” and “the” include both singular and plural aspects, unless the context clearly indicates otherwise.

Any discussion of documents, acts, materials, devices, articles or the like which has been included in the present specification is not to be taken as an admission that any or all of these matters form part of the prior art base or were common general knowledge in the field relevant to the present disclosure as it existed before the priority date of each claim of this application.

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or group of compositions of matter.

Each example described herein is to be applied mutatis mutandis to each and every other example of the disclosure unless specifically stated otherwise.

Those skilled in the art will appreciate that the disclosure is susceptible to variations and modifications other than those specifically described. It is to be understood that the disclosure includes all such variations and modifications. The disclosure also includes all of the steps, features, compositions and compounds referred to or indicated in this specification, individually or collectively, and any and all combinations or any two or more of said steps or features.

The present disclosure is not to be limited in scope by the specific examples described herein, which are intended for the purpose of exemplification only. Functionally-equivalent products, compositions and methods are clearly within the scope of the disclosure.

The present disclosure is performed without undue experimentation using, unless otherwise indicated, conventional techniques of molecular biology, recombinant DNA technology, cell biology and immunology. Such procedures are described, for example, in Sambrook, Fritsch & Maniatis, Molecular Cloning: A Laboratory Manual, Cold Spring Harbor Laboratories, New York, Second Edition (1989), whole of Vols I, II, and III; DNA Cloning: A Practical Approach, Vols. I and II (D. N. Glover, ed., 1985), IRL Press, Oxford, whole of text; Oligonucleotide Synthesis: A Practical Approach (M. J. Gait, ed, 1984) IRL Press, Oxford, whole of text, and particularly the papers therein by Gait, ppl-22; Atkinson et al, pp35-81; Sproat et al, pp 83-115; and Wu et al, pp 135-151; 4. Nucleic Acid Hybridization: A Practical Approach (B. D. Hames & S. J. Higgins, eds., 1985) IRL Press, Oxford, whole of text; Immobilized Cells and Enzymes: A Practical Approach (1986) IRL Press, Oxford, whole of text; Perbal, B., A Practical Guide to Molecular Cloning (1984); Methods In Enzymology (S. Colowick and N. Kaplan, eds., Academic Press, Inc.), whole of series, Sakakibara, D., Teichman, J., Lien, E. Land Fenichel, R. L. (1976). Biochem. Biophys. Res. Commun. 73 336-342; Merrifield, R. B. (1963). J. Am. Chem. Soc. 85, 2149-2154; Barany, G. and Merrifield, R. B. (1979) in The Peptides (Gross, E. and Meienhofer, J. eds.), vol. 2, pp. 1-284, Academic Press, New York. 12. Wünsch, E., ed. (1974) Synthese von Peptiden in Houben-Weyls Metoden der Organischen Chemie (Müler, E., ed.), vol. 15, 4th edn., Parts 1 and 2, Thieme, Stuttgart; Bodanszky, M. (1984) Principles of Peptide Synthesis, Springer-Verlag, Heidelberg; Bodanszky, M. & Bodanszky, A. (1984) The Practice of Peptide Synthesis, Springer-Verlag, Heidelberg; Bodanszky, M. (1985) Int. J. Peptide Protein Res. 25, 449-474; Handbook of Experimental Immunology, Vols. I-IV (D. M. Weir and C. C. Blackwell, eds., 1986, Blackwell Scientific Publications); and Animal Cell Culture: Practical Approach, Third Edition (John R. W. Masters, ed., 2000), ISBN 0199637970, whole of text.

Throughout this specification, unless the context requires otherwise, the word “comprise”, or variations such as “comprises” or “comprising”, will be understood to imply the inclusion of a stated step or element or integer or group of steps or elements or integers but not the exclusion of any other step or element or integer or group of elements or integers.

Unless specifically defined otherwise, all technical and scientific terms used herein shall be taken to have the same meaning as commonly understood by one of ordinary skill in the art (for example, in cell culture, molecular genetics, immunology, immunohistochemistry, protein chemistry, and biochemistry).

The term “consists of” or “consisting of” shall be understood to mean that a method, process or composition of matter has the recited steps and/or components and no additional steps or components.

The term “about”, as used herein when referring to a measurable value such as an amount of weight, time, dose, etc. is meant to encompass variations of ±20% or ±10%, more preferably ±5%, even more preferably ±1%, and still more preferably ±0.1% from the specified amount, as such variations are appropriate to perform the disclosed method.

The term AMH as used herein refers to anti-mullerian hormone. This term can be used interchangeably with the term mullerian-inhibiting substance (MIS). The term “pre-pro protein” as used herein refers to the full length protein including the leader sequence, for example the sequence set forth in SEQ ID NO:1 (wild-type AMH protein). The term “proprotein” or “prodomain” as used herein refers to the AMH protein sequence lacking the leader sequence, for example the sequence from amino acid residues Arg26 to Gly447. The term “mature” AMH protein or polypeptide as used herein refers to the AMH polypeptide following processing and cleavage. The mature sequence is the sequence from Ser452 to Arg560. The biologically active AMH protein is a homodimer comprising two monomer units wherein each monomer unit has the sequence from Ser452 to Arg560.

It will be understood that the AMH analogues described herein are in isolated form. By “isolated” it is meant a polypeptide, polynucleotide, vector or cell that is in a form not found in nature. Isolated polypeptides, polynucleotides, vectors, or cells include those which have been purified to a degree that they are no longer in a form in which they are found in nature. In some aspects, a polypeptide, polynucleotide, vector, or cell that is isolated is substantially pure.

The term “specifically binds” as used herein when referring to an AMH analogue refers to an AMH analogue that recognises and binds to AMHR2 but that does not substantially recognise and bind other molecules in a sample.

The term “identity” or “sequence identity” and grammatical variations thereof, mean that two or more referenced entities are the same. Thus, where two polypeptide sequences are identical, they have the same amino acid sequence, at least within the referenced region or portion. Where two nucleic acid sequences are identical, they have the same polynucleotide sequence, at least within the referenced region or portion. The identity can be over a defined area (region or domain) of the sequence. The % identity is calculated by comparing two optimally aligned sequences over the window of comparison, determining the number of positions at which the identical nucleic acid base or amino acid residue occurs in both sequences to yield the number of matched positions, dividing the number of matched positions by the total number of positions in the comparison window and multiplying the result by 100 to yield the percentage of sequence identity. The % identity can be determined by GAP (Needleman and Wunsch, J. Mol Biol. 48: 444-453.1970) analysis (GCG program), the homology algorithm of Smith and Waterman (Adv. Appl. Math. 2:482 (19801), or the method of Pearson and Lipman (PNAS USA 85:2444-48 (1988). Another algorithm that is suitable for determining the percent sequence identity and sequence similarity is the BLAST algorithm which is described by Altschul et al. J. Mol. Biol. 215:403-410 (1990).

The term “increased” as used herein refers to an increase relative to a reference level, for example the native AMH polypeptide. The increase may be at least 10%, at least 20%, at least 30%, at least 40%, at least 50%, at least 60%, at least 70% or more, including, for example, at least 2-fold, at least 3-fold, at least 4-fold, at least 5-fold or greater. The increase can refer to expression level, potency or protein activity.

The term “polynucleotide” as used herein refers to a molecule of greater than about 100 nucleobases in length. A nucleobase includes for example, a naturally occurring purine or pyrimidine base found in DNA (e.g. adenosine “A”, a guanine “G”, a thymine “T”, or a cytosine “C”) or RNA (e.g. an A, a G a uracil “U” or C).

The term “viral vector” as used herein refers to the use of viruses or virus-associated vectors as carriers of the nucleic acid construct into the cell. Constructs may be integrated and packaged into non-replicating, defective viral genomes like Adenovirus, Adeno-associated virus (AAV), or Herpes simplex virus (HSV) or others, including retroviral and lentiviral vectors, for infection or transduction into cells. The vector may or may not be incorporated into the cells genome. The constructs may include viral sequences for transfection, if desired. Alternatively, the construct may be incorporated into vectors capable of episomal replication, e.g., EPV and EBV vectors.

The term “pharmaceutical composition”, as used herein, means any composition, which contains at least one therapeutically or biologically active agent and is suitable for administration to the patient. Any of these formulations can be prepared by well-known and accepted methods of the art. See, for example, Gennaro, A. R., ed., Remington: The Science and Practice of Pharmacy, 20th Edition, Mack Publishing Co., Easton, Pa. (2000).

The phrase “pharmaceutically acceptable” is employed herein to refer to those compounds, materials, compositions, and/or dosage forms that are, within the scope of sound medical judgment, suitable for use in contact with the tissues of human beings and animals without excessive toxicity, irritation, allergic response, and/or other problem or complication, commensurate with a reasonable benefit/risk ratio.

As used herein, the term “treating” includes alleviation of symptoms associated with a specific disorder or condition. “Treatment” can also mean prolonging survival as compared to expected survival if not receiving treatment. Those in need of treatment include those already with the condition or disorder as well as those prone to have the condition or disorder or those in which the condition or disorder is to be prevented.

As used herein, the term “prevention” includes prophylaxis of the specific disorder or condition. The term “preventing” refers to the avoidance or delay in manifestation of one or more symptoms or measurable markers of a disease or disorder (e.g., POA or DOR). The term includes not only the avoidance or prevention of a symptom or marker of the disease but also a reduced severity or degree of any one of the symptoms or markers of the disease, relative to those symptoms or makers in a control or non-treated individual with a similar likelihood or susceptibility of developing the disease or disorder, or relative to symptoms or markers likely to arise based on historical or statistical measures of populations affected by the disease or disorder. Reduced severity is meant at least a 10% reduction in the severity or degree of a symptom or measurable disease marker, relative to a control or reference e.g. at least 15%, 20%, 30%. 40%, 50%, 60%, 70%, 80%, 90%, 95%, 99% or even 100% (i.e. no symptoms or measurable markers).

The term “therapeutically effective amount” shall be taken to mean a sufficient quantity of AMH analogue or polynucleotide as described herein to alleviate at least one or more symptoms of the disease or disorder and relates to a sufficient amount of composition to provide the desired effect or to provide a significant reduction in a symptom or clinical marker associated with a disorder. The efficacy of treatment can be assessed in animal models of fertility and any treatment or administration of the compositions that leads to preventing pregnancy, or arresting folliculogenesis indicates effective treatment. A therapeutically or prophylactically significant reduction in a symptom is e.g. at least about 10%, at least about 20%, at least about 30%, at least about 40%, at least about 50%, at least about 75% or more in a measured parameter as compared to a control or non-treated subject.

Unless indicated otherwise, the term “wild-type” or “native” as used herein refers to the naturally occurring polypeptide or polynucleotide sequence encoding human AMH as it normally exists in vivo. As disclosed herein, the wild-type amino acid sequence for the pre-pro protein of human AMH corresponds to SEQ ID NO:1 where amino acid residues 1-25 correspond to the leader/signal sequence. As disclosed herein, the wild-type amino acid sequence for the proprotein form of AMH comprises amino acid residues 26-560 of SEQ ID NO:1 (e.g. lacking the leader sequence) which is then post-translationally processed by proteolytic cleavage to form mature AMH. As disclosed herein, the wild-type amino acid sequence for mature AMH comprises amino acid residues 452 to 560 of SEQ ID NO:1. AMH is produced as a disulphide linked homodimeric precursor which requires post-translational processing to form the active AMH. Post-translational processing includes cleavage and dissociation from the pro-domain to release mature AMH. References to variant or mutant peptides; polypeptides, or proteins described herein include peptides, polypeptides, proteins, or fragments thereof, that contain at least one amino acid residue that differs from the “wild-type” or “native” peptides; polypeptides, or proteins; i.e. include at east one amino acid residue that differs from the pre-pro protein of human AMH provided in SEQ ID NO:1 (or a fragment thereof such as amino acid residues 452 to 560 of SEQ ID NO:1). In some embodiments, the at least one amino acid residue that differs is located within amino acid residues 452 to 560 of SEQ ID NO:1 (i.e. the C-terminal domain or mature processed AMH). As the person skilled in the art would appreciate, there is at least one naturally occurring sequence polymorph of human AMH. For the purpose of the present application, variant or mutant peptides, polypeptides, or proteins include any naturally occurring sequence polymorphs of human AMH that do not have the wild-type sequence.

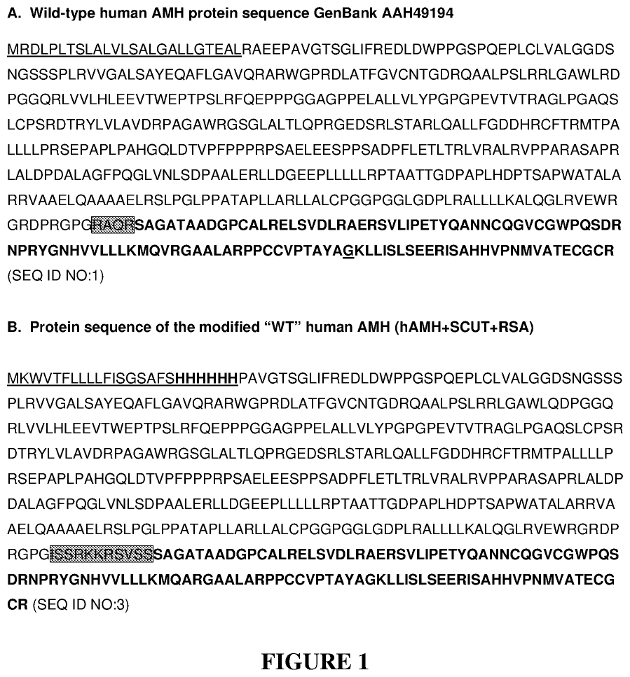

Unless indicated otherwise, residue numbering throughout the specification is with reference to the human AMH precursor shown in SEQ ID NO:1 (FIG. 1A), where the first residue in the sequence provided in SEQ ID NO:1 is numbered as position 1 and the last residue in the sequence provided in SEQ ID NO:1 is numbered as position 560. In embodiments where additional amino acids are inserted in the polypeptide, for example due to inclusion of a purification tag, a different leader sequence, a different protease cleavage sequence and the like, the inserted residues are labelled based on the residue number of the wild-type residue immediately preceding the insertion and a letter of the alphabet. See, for example, Table 1 below.

TABLE 1
Numbering of amino acid insertions at the primary cleavage site.

| | | | | | | Option 5 |
|---|---|---|---|---|---|---|
| Amino | | | | | | (includes |
| Acid | Wild | Option | Option | Option | Option | SEQ ID |
| Position | Type | 1 | 2 | 3 | 4 | NO: 37) |
| 447 | G | G | G | G | G | G |
| 447a | — | I | — | I | | X₁ |
| 447b | — | S | — | S | | X₂ |
| 447c | — | S | — | S | | X₃ |
| 448 | R | R | R | R | R | R |
| 449 | A | K | K | K | K | K |
| 450 | Q | K | K | K | K | K |
| 451 | R | R | R | R | R | R |
| 451a | — | S | — | — | S | X₈ |
| 451b | — | V | — | — | V | X₉ |
| 451c | — | S | — | — | S | X₁₀ |
| 451d | — | S | — | — | S | X₁₁ |
| 452 | S | S | S | S | S | S |

In embodiments where amino acids are deleted from the polypeptide, for example due to inclusion of a different leader sequence, a different protease cleavage sequence and the like, the residues are labelled based on the residue number of the corresponding wild-type residue provided in SEQ ID NO:1.

The term “polypeptide” as used herein refers to a polymer of amino acid residues and are not limited to a minimum length. The term includes post-translational modifications of the polypeptide such as disulphide bond formation, glycosylation, acetylation, phosphorylation, proteolytic cleavage and the like. The term also encompasses a polypeptide that includes modifications such as additions and substitutions (generally conservative in nature) to the native sequence, as long as the polypeptide maintains the desired activity. These modifications can be deliberate as through site-directed mutagenesis or errors due to PCR amplification or other recombinant methods. The incorporation of non-natural amino acids, including synthetic non-native amino acids substituted amino acids or one or more D-amino acids into the polypeptide is desirable in certain situations. D-amino acid-containing polypeptides exhibit increased stability in vitro and in vivo compared to L-amino acid containing forms.

The term “conservative substitution” when describing a polypeptide refers to a change in the amino acid composition of the polypeptide that does not substantially alter the polypeptide's activity. For example, a conservative substitution refers to substituting an amino acid residues for a different amino acid residue that has similar chemical properties. A conservative substitution of a particular amino acid sequence refers to substitution of those amino acids that are not critical for polypeptide activity or substitution of amino acids with other amino acids having similar properties such that the substitution of even critical amino acids does not reduce the activity of the polypeptide. For example, the following six groups each contain amino acids that are conservative substitutions for one another 1) alanine (A), serine (S), threonine (T); 2) aspartic acid (D), glutamic acid (E); 3) asparagine (N), glutamine (Q); 4) arginine (R), lysine (K); 5) isoleucine (I), leucine (L), methionine (M), valine (V); and 6) phenylalanine (F), tyrosine (Y), tryptophan (W).

The term “substitution” when referring to a polypeptide refers to a change in an amino acid for a different entity, for example another amino acid. The substitution may be conservative or non-conservative.

The term “recombinant” as used herein in the context of a polynucleotide means a polynucleotide of genomic, cDNA, semisynthetic and/or synthetic origin which by virtue of its origin or manipulation is not associated with all or a portion of the polynucleotide with which it is associated in nature. The term recombinant as used herein in the context of a polypeptide means a polypeptide produced by expression of a recombinant polynucleotide.

The term “subject” as used herein refers to an animal or non-human animal. The subject may be a human to whom treatment, including prophylactic treatment, is administered with the composition of the disclosure. In some examples, the non-human animal is a companion animal, preferably a cat, dog or horse. In some examples, the subject is a non-human primate, for example, chimpanzee, cynomologous monkey, spider monkey and macaque. The subject is preferably a human female. The subject can be of child-bearing age (e.g. 20 to 35 years old), a teenager (e.g. 13-19 years old), or pre-pubescent (e.g. 6-12 years old). The female subject may be older than 35 years.

The term “analogue” as used herein with reference to polypeptide (e.g., AMH) is defined broadly means a modified polypeptide wherein one or more amino acid residues of the peptide have been substituted by other amino acid residues and/or wherein one or more amino acid residues have been deleted from the peptide and/or wherein one or more amino acid residues have been added to the peptide. The analogue is derived from the native polypeptide. In some embodiments, addition or deletion of amino acid residues can take pace at the IN-terminal of the polypeptide and/or at the C-terminal of the polypeptide and/or at the N-terminal of the polypeptide of a domain of the polypeptide and/or at the C-terminal of the polypeptide of a domain of the polypeptide. The added and/or substituted amino acid residues can either be codable amino add residues or other naturally occurring residues or purely synthetic; amino acid residues. In some embodiments, the analogue is an agonist.

In nature, some polypeptides are produced as complex precursors which, in addition to targeting labels such as signal peptides, also contain other fragments of peptides which are removed (processed) at some point during protein maturation, resulting in a mature form of the polypeptide that is different from the primary translation product (aside, from the removal of the signal peptide). The term “mature” as used herein with reference to proteins refers to a post-transitionally processed polypeptide; i.e., one from which any pre- or propeptides present in the primary translation product have been cleaved or removed. The terms “precursor protein” or “prepropeptide” or “preproprotein” as used herein all refer to the primary product of translation of mRNA; i.e., with pre- and propeptides still present. “Pre” in this nomenclature generally refers to the signal peptide. The form of the translation product with only the signal peptide removed but no further processing is called a “propeptide” or “proprotein.” The fragments or segments to be removed may themselves also be referred to as “propeptides.” A proprotein or propeptide thus has had the signal peptide removed, but contains propeptides (here referring to propeptide segments) and the portions that will make up the mature protein. With reference to AMH, “mature” AMH is also referred to as the “C-terminal domain” and the propeptide segment is also referred to as the “N-terminal domain”. The C-terminal domain may comprise a sequence selected from the group consisting of SEQ ID NO:9 to SEQ ID NO:15. As would be appreciated by the skilled person, the C-terminal domain may optionally comprise additional residues at the N or C-terminus. In one embodiment, the C-terminal domain may optionally comprise SVSS at the N-terminus (e.g. SEQ ID NO:36). Similarly, the N-terminal domain may optionally comprise additional residues at the N or C-terminus.

The term “processed” as used herein with reference to AMH (i.e. “processed AMH”) refers to mature AMH (i.e. 452 to 560 of SEQ ID NO:1) following processing of the primary translation product (i.e. the preproprotein) to cleave the pre and pro domains as discussed herein.

As used herein, the term “domain” with reference to a polypeptide is defined broadly and refers to a polypeptide or a region, fragment or segment of a polypeptide. Preferably, the polypeptide or region, fragment or segment of a polypeptide forms a compact three-dimensional structure that can, for example, be independently stable and folded. However, the person skilled in the art would understand that some domains or parts thereof can be unstructured or have random coil structure. Many proteins only contain a single domain, while others may have several domains.

As used herein with reference to the AMH analogue, the term “activity” refers to the ability of the analogue to induce signalling by the AMH receptor complex. For example, in some embodiments, activity is measured by measuring Smad-1/5 response following treatment with the AMH analogue.

### Anti-Mullerian Hormone

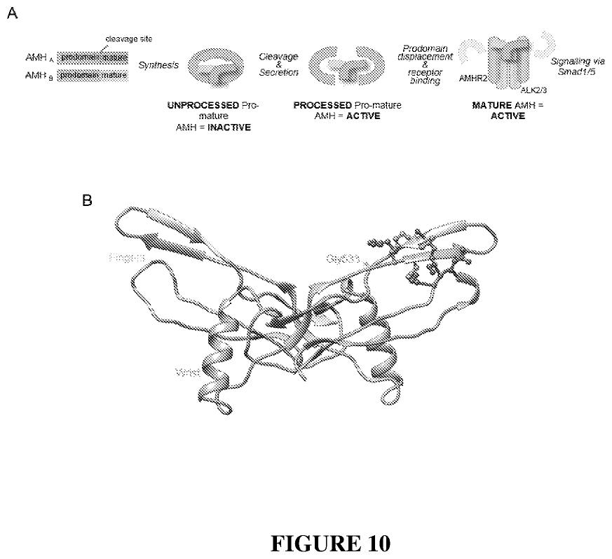

Anti-Mullerian hormone (AMH), also known as mullerian inhibiting substance (MIS) is produced as a dimeric precursor and undergoes posttranslational processing for activation requiring cleavage and dissociation from the N-terminal (pro) domain to release bioactive C-terminal fragments (see FIG. 10A).

AMH levels decline and follicle stimulating hormone (FSH) levels increase as women age. The US Centre for Human Reproduction has established age-specific levels of AMH and FSH to assess a woman's ovarian reserve. These baseline levels are as follows in Table 2:

TABLE 2
Age-specific levels of AMH and FSH

| | | |
|---|---|---|
| Age | FSH | AMH |

| | | | |
|---|---|---|---|
| <33 | Years | <7.0 mIU/mL | =2.1 ng/mL |
| 33-37 | Years | <7.9 mIU/mL | =1.7 ng/mL |
| 38-40 | Years | <8.4 mIU/mL | =1.1 ng/mL |
| =41+ | Years | <8.5 mIU/mL | =0.5 ng/mL |

AMH is a 140 kDa disulphide-linked glycoprotein member of the large transforming growth factor-β(TGF-β) multigene family of glycoproteins. Western blot analysis under reducing conditions indicates that AMH is a disulfide-linked dimer with each “monomer” cleaved into two smaller species, perhaps during the biosynthetic and secretion processes. After post-translational processing, AMH comprises a 57 kDa N-terminal domain dimer and a 12.5 kDa carboxy-terminal (C-terminal) domain dimer which together form a non-covalent complex. The C-terminal domain is the biologically active moiety and cleavage is required for activity. The N-terminal domain may assist with protein folding in vivo and facilitate delivery of the C-terminal peptide to its receptor, e.g., AMHR2.

The AMH prohormone can be cleaved by members of the subtilisin/kexin-like proprotein convertase (PC) family (e.g. furin) to generate the N-terminal domain (i.e. the prodomain) and the C-terminal domain (i.e. mature AMH). This proteolytic process is required for its physiological activity and occurs at a site in a position similar to the dibasic cleavage site found in the sequence of TGF-β. A non-cleavable mutant of AMH is biologically inactive.

Processing of the AMH precursor involves the proteolytic cleavage and removal of the leader sequence (e.g., amino acids 1-25 of SEQ ID NO: 1), the cleavage of the AMH protein to generate the N-terminal and C-terminal domains, and dissociation of C-terminal domain, which is disulfide linked to a second C-terminal domain to form the bioactive homodimer AMH protein. The mature AMH dimer is non-covalently associated with the prodomain dimer.

Cleavage occurs primarily at a kex-like site characterised by RAQR (448 to 451 of SEQ ID NO:1). The peptide bond after the second arginine is cleaved. Cleavage at this site produces the C-terminal domain of AMH (e.g., amino acids 452-560 of SEQ ID NO: 1). As used herein, the term “proprotein convertase site” refers to the primary AMH cleavage site and comprises residues 448 to 451 (RAQR) of SEQ ID NO:1.

A secondary cleavage site (referred to as “R/S”), whose significance is unknown is observed less frequently at residues 254-255 of SEQ ID NO:1. This site contains an R S, but otherwise does not follow the consensus Arg-X-(Arg/Lys)-Arg for furin cleavage.

Non-cleavable mutants of AMH are not biologically active and mutations in the human gene that truncate the carboxy-terminal domain lead to persistent Mullerian duct syndrome. The role of the amino terminal domain in vivo may assist in protein folding and to facilitate delivery of the C-terminal domain fragment to its receptor.

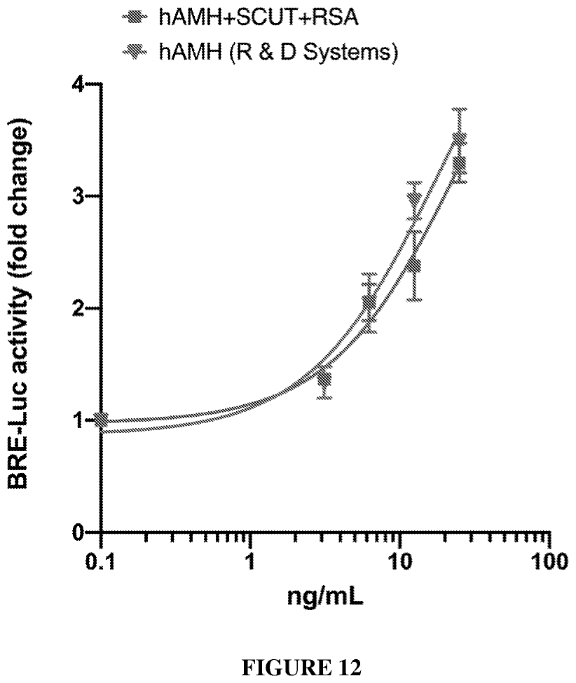

In some examples, the primary RAQR cleavage site at amino acid position 448-451 of SEQ ID NO:1 is replaced with a consensus sequence for the subtilisin/kexin-like proprotein convertase (PC) family, for example as described in Duckert, Brunark & Blom (2004) Protein Eng Des Sel, 17:107-112. In some examples, the primary cleavage site at amino acid position 448-451 of SEQ ID NO:1 is replaced by the furin consensus sequence, for example the sequence motif R-X-[K/R]-R↓ where X is any amino acid residue. In some examples, the primary RAQR cleavage site at amino acid position 448-451 of SEQ ID NO:1 is changed to RARR as described in PCT application PCT/US14/024010. In some examples, the primary cleavage site at amino acid position 448-451 of SEQ ID NO:1 is changed to X₁X₂X₃RKKRX₈X₉X₁₀X₁₁ (SEQ ID NO:37), wherein X₁ is absent or isoleucine, X₂ is absent or serine, X₃ is absent or serine, X₈ is absent or serine, X₉ is absent or valine, X₁₀ is absent or serine and X₁₁ is absent or serine. In some examples, the primary RAQR cleavage site at amino acid position 448-451 of SEQ ID NO:1 is changed to RKKR (SEQ ID NO:38). In some examples, the primary cleavage site at amino acid position 448-451 of SEQ ID NO:1 is changed to ISSRKKRSVSS (SEQ ID NO:6; also referred to herein as SCUT). In some examples, the primary cleavage site at amino acid position 448-451 of SEQ ID NO:1 is changed to ISSRKKR (SEQ ID NO:39). In some examples, the primary cleavage site at amino acid position 448-451 of SEQ ID NO:1 is changed to RKKRSVSS (SEQ ID NO:40). Inclusion or omission of any one or more of amino acids X₁ to X₃ and X₈ to X₁₁ is also within the scope of the disclosure. It should be noted that the use of a full-length SCUT i.e. Option 1 in Table 1, leaves an N-terminal SVSS on the mature AMH because cleavage occurs between R451 and the next S downstream (i.e. S451a). The activity of mature processed AMH produced from hAMH+SCUT+RSA is comparable to the activity of hAMH purchased from R&D Systems (FIG. 12). If a truncated SCUT is used (see, for example, the various options in Table 1), this could alter the presence, sequence and/or number of the additional N-terminal amino acids. For example, in Options 2 or 3 of Table 1, the SVSS will be entirely absent. Other options and outcomes will be obvious to the skilled person.

As discussed above, the mature wild-type AMH protein is initially produced as a precursor comprising a N-terminal leader sequence, which corresponds to amino acid residues 1-25 of wild-type AMH protein of SEQ ID NO: 1. This leader sequence is cleaved off to produce the AMH proprotein. In all aspects of the disclosure, the AMH precursor can have a non-endogenous leader/signal sequence, where the leader/signal sequence of amino acids 1-25 of SEQ ID NO: 1 has been replaced with different leader sequence, such as, for example, a human serum albumin leader sequences. In all aspects of the disclosure, the AMH precursor as disclosed herein can be a modified recombinant AMH polypeptide where the primary RAQR cleavage site is replaced by a furin consensus sequence and where the endogenous leader/signal sequence has been replaced with a heterologous leader sequence. In all aspects of the disclosure, the AMH precursor as disclosed herein can be a modified recombinant AMH polypeptide where the primary RAQR cleavage site is replaced by SEQ ID NO:6 and where the endogenous leader/signal sequence has been replaced with a heterologous leader sequence.

Secreted proteins are expressed initially inside the cell in a precursor form containing a leader sequence ensuring entry into the secretory pathway. Such leader sequences, also referred to as signal peptides, direct the expressed product across the membrane of the endoplasmic reticulum (ER). Signal peptides are generally cleaved off by signal peptidases during translocation to the ER. Once entered in the secretory pathway, the protein is transported to the Golgi apparatus. From the Golgi the protein can follow different routes that lead to compartments such as the cell vacuole or the cell membrane, or it can be routed out of the cell to be secreted to the external medium (Pfeffer and Rothman (1987) Ann. Rev. Biochem. 56:829-852).

In some examples, the AMH analogue comprises a modified leader/signal sequence in place of the wild-type leader sequence of the AMH protein corresponding to amino acid residues 1-25 of SEQ ID NO:1. In some examples, the native leader sequence of amino acid residues 1-25 of SEQ ID NO: 1 is replaced with a heterologous leader sequence, for example, but not limited to an albumin leader sequence, or functional fragment thereof. In some examples, the heterologous leader sequence is a human serum albumin sequence (HSA), for example, a leader sequence corresponding SEQ ID NO:26. In some examples, the heterologous leader sequence is a rat serum albumin sequence, for example, a leader sequence corresponding SEQ ID NO:8.

Modified versions of HSA leader sequence are also encompassed by the disclosure, as disclosed in, for example U.S. Pat. No. 5,759,802 which is incorporated herein in its entirety by reference. In some examples, a functional fragment of HSA leader sequence is MKWVTFISLLFLFSSAYS (SEQ ID NO:17) or variations therefor, which are disclosed in EP patent EP2277889 which is incorporated herein in its entirety. Variants of the pre-pro region of the HSA signal sequence (e g., MKWVTFISLLFLFSSAYSRGVFRR, SEQ ID NO: 18) include fragments, such as the pre region of the HSA signal sequence (e.g., MKWVTFISLLFLFSSAYS, SEQ ID NO:19) or variants thereof, such as, for example, MKWVSFISLLFLFSSAYS, (SEQ ID NO: 20).

In some embodiments, the leader sequence is a leader sequence is at least about 60%, or at least about 70%, or at least about 80%, or at least about 90%, or at least about 95%, or at least about 96%, or at least about 97%, or at least about 98%, or at least about 99% identical to amino acid residues of SEQ ID NO: 7. In some embodiments, the leader sequence is a leader sequence is at least about 60%, or at least about 70%, or at least about 80%, or at least about 90%, or at least about 95%, or at least about 96%, or at least about 97%, or at least about 98%, or at least about 99% identical to amino acid residues of SEQ ID NO: 8. In some embodiments, the leader sequence is a leader sequence is at least about 60%, or at least about 70%, or at least about 80%, or at least about 90%, or at least about 95%, or at least about 96%, or at least about 97%, or at least about 98%, or at least about 99% identical to amino acid residues of SEQ ID NO: 26.

Other leader sequence are also contemplated by the disclosure to replace amino acids 1 to 25 of SEQ ID NO:1. Such leader sequences are well known in the art, and include the leader sequences comprising an immunoglobulin signal peptide fused to a tissue-type plasminogen activator propeptide (IgSP-tPA), as disclosed in US 2007/0141666, which is incorporated herein in its entirety by reference. Numerous other signal peptides are used for production of secreted proteins. One of them is a murine immunoglobulin signal peptide (IgSP, EMBL Accession No. M13331). IgSP was first identified in 1983 by Loh et al. (Cell. 33:85-93). IgSP is known to give a good expression in mammalian cells. For example. EP patent No. 0382762 discloses a method of producing horseradish peroxidase by constructing a fusion polypeptide between IgSP and horseradish peroxidase. Other leader sequences include, for example, but not limited to, the MPIF-1 signal sequence (e.g., amino acids 1-21 of GenBank Accession number AAB51134) MKVSVAALSCLMLVTALGSQA (SEQ ID NO:16); the stanniocalcin signal sequence (MLQNSAVLLLLVISASA, SEQ ID NO:17); the invertase signal sequence (e g, MLLQAFLFLLAGFAAKISA, SEQ ID NO:18); the yeast mating factor alpha signal sequence (e.g., K. lactis killer toxin leader sequence); a hybrid signal sequence (e.g., MKWVSFISLLFLFSSAYSRSLEKR, SEQ ID NO:19); an HSA/MFa-1 hybrid signal sequence (also known as HSA kex2) (e.g., MKWVSFISLLFLFSSAYSRSLEKR, SEQ ID NO:20); a K. lactis killer/MFa-1 fusion leader sequence (e.g., MNIFYIFLFLLSFVQGSLDKR, SEQ ID NO:21); the Immunoglobulin Ig signal sequence (e.g., MGWSCIILFLVATATGVHS, SEQ ID NO:22); the Fibulin B precursor signal sequence (e.g., MERAAPSRRVPLPLLLLGGLALLAAGVDA, SEQ ID NO:23); the clusterin precursor signal sequence (e.g., MMKTLLLFVGLLLTWESGQVLG, SEQ ID NO:24); and the insulin-like growth factor-binding protein 4 signal sequence (e.g., MLPLCLVAALLLAAGPGPSLG, SEQ ID NO:25).

In further examples, the AMH precursor or a nucleic acid sequence encoding the same also comprises a tag to aid purification. In some examples, the tag can be a c-myc, a polyhistidine or FLAG tag. In some examples, the tag is a polyhistidine tag. In certain examples, the tag is located so that after posttranslational processing (e.g., cleavage with furin or a similar protease) the C-terminal domain fragment is not tagged. In other words, the tag is located so that after posttranslational processing (e.g., cleavage with furin or a similar protease) the N-terminal domain is tagged. In certain examples, the polyhistidine tag is located following the leader/signal sequence, for example after amino acid residue 25 of SEQ ID NO:1. In certain examples, the polyhistidine tag is located immediately before amino acid residue 30 of SEQ ID NO:1. The polyhistidine tag may be His6 or His8. The AMH precursor may comprise more than one tag which may be the same or different. Preferably, the tags do not interfere or substantially affect the bioactivity of the AMH analogue function at binding and activating AMHR2.

In further examples, the AMH analogue is glycosylated on one or more residues. In some examples, the AMH analogue is glycosylated on one or more residues in the N-terminal domain.

In some embodiments, the AMH analogue comprises at least one amino acid residue modification relative to a native human AMH polypeptide set forth in SEQ ID NO:5. In some embodiments, the at least one amino acid modification is in the putative finger domains of AMH (FIG. 10B). In some embodiments, the at least one amino acid modification is in putative finger 2 of AMH (FIG. 10B). In some embodiments, the modification is present within amino acid residues 533 to 548 of SEQ ID NO:1. In some embodiments, the modification is present within amino acid residues 533 to 535 of SEQ ID NO:1. In some embodiments, the modification is at amino acid residue 533 of SEQ ID NO:1. In some embodiments, the modification is at amino acid residue 535 of SEQ ID NO:1. In some embodiments, the modification is at amino acid residue 533 and 535 of SEQ ID NO:1.

In some embodiments, the AMH analogue comprises at least one C-terminal domain polypeptide comprising an amino acid sequence which has at least 80% identity to amino acid residues 452 to 560 of SEQ ID NO:1, and at least one N-terminal domain polypeptide comprising an amino acid sequence which has at least 80% identity to amino acid residues 30 to 447 of SEQ ID NO:1. In one example, the AMH analogue comprises two C-terminal domain polypeptides comprising an amino acid sequence which has at least 80% identity to amino acid residues 452 to 560 of SEQ ID NO:1, and two N-terminal domain polypeptides comprising an amino acid sequence which has at least 80% identity to amino acid residues 30 to 447 of SEQ ID NO:1.

The AMH analogue is able to modulate activity of the AMHR complex. In some embodiments, the AMH analogue is an agonist of AMHR2.

The AMH sequence from various non-human animals are provided in Table 3 below.

TABLE 3
AMH sequences

| | Reference to | Reference to |
|---|---|---|
| | polynucleotide | polypeptide |
| Species | sequence | sequence |
| Mouse (Mus musculus) | NM_007445.3 | NP_031471.2 |
| | [SEQ ID NO: 27] | [SEQ ID NO: 2] |
| Horse (Equus caballus) | NM_001317263.1 | NP_001304192.1 |
| | [SEQ ID NO: 28] | [SEQ ID NO: 31] |
| Dog (Canis lupis familiaris) | NM_001314127.1 | NP_001301056.1 |
| | [SEQ ID NO: 29] | [SEQ ID NO: 32] |
| Cat (Felis catus) | XM_011288073.3 | XP_011286375.2 |
| | [SEQ ID NO: 30] | [SEQ ID NO: 33] |

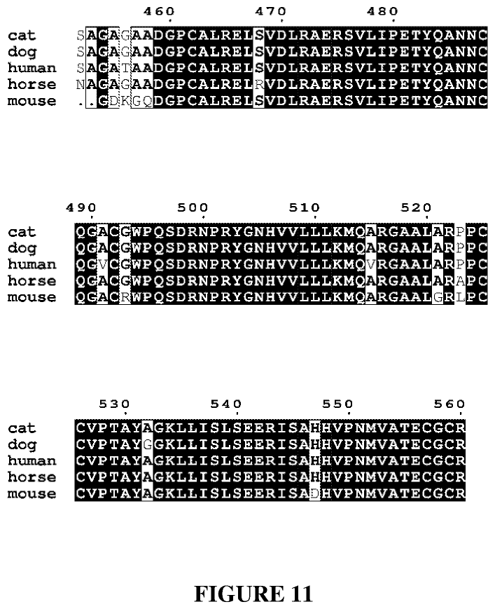

An alignment of the mature AMH sequence from human and non-human animals is provided in FIG. 11.

The AMH analogues described herein may also be further modified, for instance, by glycosylation, amidation, carboxylation, or phosphorylation, or by the creation of acid addition salts, amides, esters, in particular C-terminal esters, and N-acyl derivatives. In some embodiments, the AMH analogue polypeptide or its corresponding precursor can be fused to one or more fusion partners. In one example, the fusion partner is an Fc protein (e.g. animal or human Fc). The fusion protein may further include a second fusion partner such as a purification or detection tag, for example, proteins that may be detected directly or indirectly (such as green fluorescent protein, hemagglutinin, or alkaline phosphatase; DNA binding domains (e.g. GAL4 or LexA); gene activation domains (GAL4 or VP16), purification tags, or secretion signal peptides (e.g. preprotrypsin signal sequence).

In some examples, the AMH analogue polypeptide is fused to a second fusion partner such as a carrier molecule to enhance its bioavailability. Such carriers are known in the art and include poly (alkyl) glycol such as poly ethylene glycol (PEG). Other modifications contemplated include attachment to a polymer (e.g. PEG). Methods of PEGylation are known in the art. Other examples include conjugation or genetic fusion with transferrin, albumin, growth hormone or cellulose or other molecule which improve the pharmacokinetics of the polypeptide.

In certain examples, the AMH analogue may be modified to increase the half-life of the analogue. In some examples, the AMH analogue comprises or is conjugated to a half-life extending moiety which increases half-life of the AMH analogue in vivo. Suitable half-life extending moieties include, but are not limited to polyethylene glycol, lipids and proteins (e.g., Fc fragment, albumin binding proteins, polypeptides comprising as PAS sequence, XTEN). Fusion to serum albumin can also increase the serum half-life of the polypeptides. As understood by the person skilled in the art, increased, or extended, half-life means slowed clearance of a particular molecule from blood.

A half-life extending moiety may for example comprise a peptide or protein that will allow in vivo association to serum albumins. In particular, the half-life extending moiety may be an albumin binding moiety. An albumin binding moiety may e.g. consist of a naturally occurring polypeptide, or an albumin binding fragment thereof, or an engineered polypeptide. An engineered albumin binding polypeptide may for example be a variant of a protein scaffold, which variant has been selected for its specific binding affinity for albumin. In a specific embodiment, the protein scaffold may be selected from domains of streptococcal Protein C or derivatives thereof. Other examples of suitable albumin binding domains are disclosed in WO2009/016043.

A half-life extending moiety may for example comprise a polypeptide-based, random-coil domain. In some embodiments, the half-life extending moiety may be a PAS polypeptide (see, or example, Payne et al. (2010) Pharm. Dev. Technol., 1-18; Pisal et al. (2010) J. Pharm. Sci. 99 (6), 2557-2575; Veronese. (2001) Biomaterials 22 (5), 405-417; PCT/EP2011/058307 and PCT/EP2008/005020). PAS polypeptides contain sequences of praline, alanine, and optionally serine (PA/S or PAS) residues which form a stably disordered polypeptide. In some embodiments, the half-life extending moiety may be a XTEN polypeptide (see, for example; Podust et. al., (2016) J Control Release 240:52-66). XTENs are composed entirely of alanine, glutamate, glycine, praline, serine, and threonine residues that form highly hydrophilic, unstructured polypeptides.

Those skilled in the art will appreciate that a number of other well-known approaches exist to extend the in vivo half-life of polypeptides and any suitable approach can be used. Example strategies for extending the half-life of a therapeutic protein are also discussed in Zaman et. al., (2019) J. Control. Release. 301:176-189.

### Preparation of Mutant AMH Polypeptides

Altered polypeptides (AMH analogues) can be prepared using any technique known in the art. For example, a polynucleotide of the invention can be subjected to in vitro mutagenesis. Such in vitro mutagenesis techniques include sub-cloning the polynucleotide into a suitable vector, transforming the vector into a “mutator” strain such as the E. coli XL-1 red (Stratagene) and propagating the transformed bacteria for a suitable number of generations. Products derived from mutated/altered DNA can readily be screened using techniques described herein to determine if they have receptor-binding activity. In some embodiments, a polynucleotide of the invention can be subjected to site-directed mutagenesis using techniques known to the person skilled in art, for example the QuikChange™ method developed by Stratagene Inc. (now Agilent technologies) or other commercially available kits/strategies.

In designing amino acid sequence mutants, the location of the mutation site and the nature of the mutation will depend on characteristic(s) to be modified. The sites for mutation can be modified individually or in series, e.g., by (1) substituting first with conservative amino acid choices and then with more radical selections depending upon the results achieved, (2) deleting the target residue, or (3) inserting other residues adjacent to the located site.

Amino acid sequence deletions generally range from about 1 to 15 residues, more preferably about 1 to 10 residues and typically about 1 to 5 contiguous residues. Substitution mutants have at least one amino acid residue in the polypeptide removed and a different residue inserted in its place. The sites of greatest interest for substitutional mutagenesis include sites identified as important for receptor binding.

In certain examples, the site(s) are substituted in a relatively conservative manner. Such conservative substitutions are shown in Table 4 under the heading of “exemplary substitutions”.

TABLE 4
Exemplary Substitutions

| Original Residue | Exemplary Substitutions |
|---|---|
| Ala (A) | val; leu; ile; gly |
| Arg (R) | lys |
| Asn (N) | gln; his |
| Asp (D) | glu |
| Cys (C) | ser |
| Gln (Q) | asn; his |
| Glu (E) | asp |
| Gly (G) | pro, ala |
| His (H) | asn; gln |
| Ile (1) | leu; val; ala |
| Leu (L) | ile; val; met; ala; phe |
| Lys (K) | arg |
| Met (M) | leu; phe |
| Phe(F) | leu; val; ala |
| Pro (P) | gly |
| Ser(S) | thr |
| Thr (T) | ser |
| Trp (W) | tyr |
| Tyr (Y) | trp; phe |
| Val (V) | ile; leu; met; phe; ala |

The amino acids described herein are preferably in the “L” isomeric form. However, residues in the D isomeric form can be substituted for any L-amino acid residue, as long as the desired functional property of binding is retained by the polypeptide. Modifications also include structural and functional analogues, for example, peptidomimetics having synthetic or non-natural amino acids or amino acid analogues and derivatized forms.

### Recombinant Production

### Vectors

The AMH analogues of the disclosure can be produced recombinantly using techniques and materials readily obtainable for example an automated peptide synthesis apparatus (see, e.g., Applied Biosystems, Foster City, Calif.).

The term “recombinant” as used herein to describe a nucleic acid molecule, means a polynucleotide of genomic, cDNA, viral, semisynthetic, and/or synthetic origin, which, by virtue of its origin or manipulation, is not associated with all or a portion of the polynucleotide with which it is associated in nature. The term recombinant as used with respect to a protein or polypeptide, means a polypeptide produced by expression of a recombinant polynucleotide. The term recombinant as used with respect to a host cell means a host cell into which a recombinant polynucleotide has been introduced. Recombinant is also used herein to refer to, with reference to material (e.g., a cell, a nucleic acid, a protein, or a vector) that the material has been modified by the introduction of a heterologous material (e.g., a cell, a nucleic acid, a protein, or a vector).

For recombinant production, the nucleic acid encoding an AMH polypeptide of the disclosure is preferably isolated and inserted into a replicable vector for further cloning (amplification of the DNA) or for expression. DNA encoding the polypeptide is readily isolated or synthesized using conventional procedures (e.g., by using oligonucleotide probes that are capable of binding specifically to DNAs encoding the polypeptide).

Many vectors are available. The vector components generally include, but are not limited to, one or more of the following: a signal sequence, a sequence encoding a polypeptide of the disclosure, an enhancer element, a promoter, and a transcription termination sequence.

The term “vector” refers to a nucleic acid molecule capable of transporting another nucleic acid to which it has been linked; a plasmid is a species of the genus encompassed by “vector”. The term “vector” typically refers to a nucleic acid sequence containing an origin of replication and other entities necessary for replication and/or maintenance in a host cell. Vectors capable of directing the expression of genes and/or nucleic acid sequence to which they are operatively linked are referred to herein as “expression vectors”. In general, expression vectors of utility are often in the form of “plasmids” which refer to circular double stranded DNA loops which, in their vector form are not bound to the chromosome, and typically comprise entities for stable or transient expression or the encoded DNA.

(i) Signal sequence component. The AMH polypeptides of the disclosure may be produced recombinantly not only directly, but also as a fusion polypeptide with a heterologous polypeptide, which is preferably a signal sequence or other polypeptide having a specific cleavage site at the N-terminus of the mature protein or polypeptide. The heterologous signal sequence selected preferably is one that is recognized and processed (i.e., cleaved by a signal peptidase) by the host cell.

(ii) Promoter component. Expression and cloning vectors usually contain a promoter that is recognized by the host organism and is operably linked to the antibody nucleic acid. Promoters suitable for use with prokaryotic hosts include the phoA promoter, β-lactamase and lactose promoter systems, alkaline phosphatase, a tryptophan (trp) promoter system, and hybrid promoters such as the tac promoter. However, other known bacterial promoters are suitable. Promoters for use in bacterial systems also will contain a Shine-Dalgarno (S. D.) sequence operably linked to the DNA encoding the polypeptide.

As used herein, a “promoter” or “promoter region” or “promoter element” used interchangeably herein, refers to a segment of a nucleic acid sequence, typically but not limited to DNA or RNA or analogues thereof, that controls the transcription of the nucleic acid sequence to which it is operatively linked. The promoter region includes specific sequences that are sufficient for RNA polymerase recognition, binding and transcription initiation. This portion of the promoter region is referred to as the promoter. In addition, the promoter region includes sequences which modulate this recognition, binding and transcription initiation activity of RNA polymerase. These sequences may be cis-acting or may be responsive to trans-acting factors. Promoters, depending upon the nature of the regulation may be constitutive or regulated. A promoter can refer to a tissue specific promoter (e.g., specific for expression on the ovary, or uterus).

Promoters are known for eukaryotes. Virtually all eukaryotic genes have an AT-rich region located approximately 25 to 30 bases upstream from the site where transcription is initiated. Another sequence found 70 to 80 bases upstream from the start of transcription of many genes is a CNCAAT region where N may be any nucleotide. At the 3′ end of most eukaryotic genes is an AATAAA sequence that may be the signal for addition of the poly A tail to the 3′ end of the coding sequence. All of these sequences are suitably inserted into eukaryotic expression vectors. Examples of suitable promoting sequences for use with yeast hosts include the promoters for 3-phosphoglycerate kinase or other glycolytic enzymes, such as enolase, glyceraldehyde-3-phosphate dehydrogenase, hexokinase, pyruvate decarboxylase, phosphofructokinase, glucose-6-phosphate isomerase, 3-phosphoglycerate mutase, pyruvate kinase, triosephosphate isomerase, phosphoglucose isomerase, and glucokinase. Other yeast promoters, which are inducible promoters having the additional advantage of transcription controlled by growth conditions, are the promoter regions for alcohol dehydrogenase 2, isocytochrome C, acid phosphatase, degradative enzymes associated with nitrogen metabolism, metallothionein, glyceraldehyde-3-phosphate dehydrogenase, and enzymes responsible for maltose and galactose utilization. Suitable vectors and promoters for use in yeast expression are further described in EP 73,657. Yeast enhancers also are advantageously used with yeast promoters.

(iii) Enhancer element component. Transcription of a DNA encoding an AMH polypeptide of the disclosure by higher eukaryotes is often increased by inserting an enhancer sequence into the vector. Many enhancer sequences are now known from mammalian genes (globin, elastase, albumin, α-fetoprotein, and insulin). Typically, however, one will use an enhancer from a eukaryotic cell virus. Examples include the SV40 enhancer on the late side of the replication origin (bp 100-270), the cytomegalovirus early promoter enhancer, the polyoma enhancer on the late side of the replication origin, and adenovirus enhancers. See also Yaniv (1982) Nature 297: 17-18 on enhancing elements for activation of eukaryotic promoters. The enhancer may be spliced into the vector at a position 5′ or 3′ to the AMH polypeptide encoding sequence, but is preferably located at a site 5′ from the promoter.

(iv) Transcription termination component. Expression vectors used in eukaryotic host cells (yeast, fungi, insect, plant, animal, human, or nucleated cells from other multicellular organisms) will also contain sequences necessary for the termination of transcription and for stabilizing the mRNA. Such sequences are commonly available from the 5′ and, occasionally 3′, untranslated regions of eukaryotic or viral DNAs or cDNAs. These regions contain nucleotide segments transcribed as polyadenylated fragments in the untranslated portion of the mRNA encoding the antibody. One useful transcription termination component is the bovine growth hormone polyadenylation region. See WO94/11026 and the expression vector disclosed therein.

(v) Selection and transformation of host cells. Suitable host cells for cloning or expressing the DNA in the vectors herein are the prokaryote, yeast, or higher eukaryote cells described above. Suitable prokaryotes for this purpose include eubacteria, such as Gram-negative or Gram-positive organisms, for example, Enterobacteriaceae such as Escherichia, e.g., E. coli, Enterobacter, Erwinia, Klebsiella, Proteus, Salmonella, e.g., Salmonella typhimurium, Serratia, e.g., Serratia marcescans, and Shigella, as well as Bacilli such as B. subtilis and B. licheniformis, Pseudomonas such as P. aeruginosa, and Streptomyces. One preferred E. coli cloning host is E. coli 294 (ATCC 31,446), although other strains such as E. coli B, E. coli X 1776 (ATCC 31,537), and E. coli W3110 (ATCC 27,325) are suitable. These examples are illustrative rather than limiting.

Other expression vectors that may be useful herein include, but are not limited to, plasmids, episomes, bacterial artificial chromosomes, yeast artificial chromosomes, bacteriophages or viral vectors, and such vectors can integrate into the hosts genome or replicate autonomously in the particular cell. A vector can be a DNA or RNA vector. Other forms of expression vectors known by those skilled in the art which serve the equivalent functions can also be used, for example self-replicating extrachromosomal vectors or vectors which integrates into a host genome Preferred vectors are those capable of autonomous replication and/or expression of nucleic acids to which they are linked. Vectors capable of directing the expression of genes to which they are operatively linked are referred to herein as “expression vectors”. Expression vectors can result in stable or transient expression of the DNA. An exemplary expression vector for use in the present disclosure is pcDNA3.1.

In some examples, the nucleic acid encoding the AMH analogue is administered as a viral vector. Such viral vectors are suitable for use in gene therapy as described for example in U.S. Pat. No. 5,399,346. Entry into the cell can be facilitated by means known in the art such as providing the polynucleotide in a vector or by encapsulation of the polynucleotide in a liposome.

Expression vectors comparable with eukaryotic cells can be used to produce recombinant constructs for expression of an AMH analogue as described herein. Eukaryotic cell expression vectors are known in the art and are available from commercial sources. Such vectors are typically provided with restriction sites for insertion of the DNA. These vectors can be viral vectors, for example, adenovirus, adeno-associated virus, pox virus such as orthopox (e.g. vaccinia), avipox, lentivirus, or murine Maloney leukemia virus.

In some examples, a plasmid expression vector may be used. Plasmid expression vectors include, pcDNA3.1, pET vectors, pGEX vectors and pMAL vectors for protein expression in E. coli host cells such as BL21, AD494(DE3)pLys, Rosetta (DE3), Origami (DE3) and pCIneo. In some examples, the vector is a replication incompetent adenoviral vector, for example, pAdeno X, pAd5F35, pLP-Adeno-X-CMV (Clontech®), pAd/CMVN5-DEST, pAd-DEST vector (Invitrogen™ Inc.).

Viral vector systems which can be utilized in the present invention include, but are not limited to (a) adenovirus vectors; (b) retrovirus vectors; (c) adeno-associated virus vectors; (d) herpes simplex virus vectors; (e) SV 40 vectors; (f) polyoma virus vectors; (g) papilloma virus vectors; (h) picornavirus vectors; (i) pox virus vectors such as an orthopox, e.g., vaccinia virus vectors or avipox, e.g. canary pox or fowl pox; and (j) a helper-dependent or gutless adenovirus. In one example, the vector is an adenovirus. Replication-defective viruses can also be advantageous.

As used herein, the term “adeno-associated virus (AAV) vector” means an AAV viral particle containing an AAV vector genome (which, in turn, comprises the first and second expression cassettes referred to herein). Suitable AAV vectors are known to the person skilled in the art and includes AAV vectors of all serotypes, for example AAV-1 through AAV-10, such as preferably AAV-1 (U.S. Pat. No. 6,759,237), AAV-2, AAV-4, AAV-5, AAV-6, AAV-7, AAV-8, AAV-9, and combinations thereof. See for example the publication of International Patents Nos. WO 02/33269, WO 02/386122 (AAV8), and GenBank, and how such sequences have been altered to correct singlet errors, for example, AAV6.2, AAV6.1, AAV6.1.2, rh64R1 and rh8R (see, for example, WO 2006/110689, published Oct. 19, 2006.) Alternatively, other AAV sequences including those identified by one skilled in the art with the use of known techniques (see, for example, Patent publication International No. WO 2005/033321 and GenBank) or by other means, may be modified as described herein.

The vector may or may not be incorporated into the cells genome. The constructs may include viral sequences for transfection, if desired. Alternatively, the construct may be incorporated into vectors capable of episomal replication, e.g., EPV and EBV vectors.

### Host Cells

Suitable host cells for the expression of the AMH polypeptides are derived from multicellular organisms. Examples of invertebrate cells include plant and insect cells. Numerous baculoviral strains and variants and corresponding permissive insect host cells from hosts such as Spodoptera frugiperda (caterpillar), Aedes aegypti (mosquito), Aedes albopictus (mosquito), Drosophila melanogaster (fruitfly), and Bombyx mori have been identified. A variety of viral strains for transfection are publicly available, e.g., the L-I variant of Autographa californica NPV and the Bm-5 strain of Bombyx mori NPV, and such viruses may be used as the virus herein according to the present invention, particularly for transfection of Spodoptera frugiperda cells.

Examples of useful mammalian host cell lines are monkey kidney CVI line transformed by SV40 (COS-7, ATCC CRL 1651); human embryonic kidney line (293 or 293 cells subcloned for growth in suspension culture, Graham et al. (1977) Gen Virol. 36:59); baby hamster kidney cells (BHK, ATCC CCL 10); Chinese hamster ovary cells (CHO, Urlaub et al. (1980) Proc. Natl. Acad. Sci USA 77:4216); mouse Sertoli cells (TM4, Mather (1980) Biol. Reprod. 23:243-251); monkey kidney cells (CVI ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumour (MMT 060562, ATCC CCL51); TRI cells (Mather et al. (1982) Annals N. Y. Acad. Sci. 383:44-68); MRC 5 cells; FS4 cells; and PER.C6™ (Crucell Nev.).

In certain examples, the AMH analogue described herein can be isolated and/or purified or substantially purified by one or more purification methods described herein or known by those skilled in the art. Generally, the purities are at least 90%, in particular 95% and often greater than 99%.

In certain embodiments, the naturally occurring compound is excluded from the general description of the broader genus.

### Measuring Activity of AMH Analogues

As used herein with reference to the AMH analogue, the term “activity” refers to the ability of the analogue to induce signalling by the AMH receptor complex. The AMH analogues described herein may have activity that is comparable to, or greater than the activity of native human AMH. In some embodiments, the AMH analogues described herein may be able to activate AMHR2 at a level that is comparable to, or greater than the level of activation caused by native human AMH. In some embodiments, the AMH analogues described herein may be able to activate AMHR1 at a level that is comparable to, or greater than the level of activation caused by native human AMH. In some embodiments, the AMH analogues described herein may be able to activate SMAD1/5 (e.g. induce phosphorylation of SMAD1/5) at a level that is comparable to, or greater than the level of activation caused by native human AMH. The activity of the AMH analogues may be determined using techniques known to the person skilled in the art. For example, in some embodiments, activity is measured by measuring Smad-1/5 response following treatment with the AMH analogue. In some embodiments, the activity of the AMH analogues may be determined using the luciferase assay as described herein. Briefly, COV434 cells are transfected with the Smad1/5-responsive BRE-luciferase reporter and AMHR2. Twenty four (24) hours post transfection, the cells are treated overnight with the AMH analogue at a range of concentrations. The cells are harvested, lysed and luminescence is measured immediately after the addition of the substrate D-luciferin. Luciferase activity is analysed as the fold-change related to baseline activity.

### Therapeutic Uses of AMH Analogues

The AMH analogues described herein may be useful for ovarian and/or uterine protection. As used herein, “ovarian protection” refers to the protection against deleterious or adverse effects on one or both ovaries as a result of trauma, damage or the effect of an exogenous agent, e.g., a therapeutic agent or treatment. In some embodiments, an exogenous agent can be a chemotherapeutic agent or cytotoxic agent. “Ovarian protection” can also refer to the protection against any insult or trauma to the ovaries (e.g., an engraftment, or an injury). Ovarian protection can refer to the protection of one or both ovaries. “Ovarian protection” refers to protecting the function of the ovaries (e.g., produce reproductive hormones, maintain proper levels of follicle stimulating hormone, or follicle production), and the histology of the ovaries (e.g., size, and tissue health). Ovarian protection maintains at least 99%, at least 95%, at least 90%, at least 80%, at least 70%, at least 60%, at least 50%, at least 40%, at least 30%, at least 20%, at least 10% of the ovarian function following administration of a therapeutic agent of treatment, as compared to the ovarian function prior to said administration. Ovarian protection also encompasses protection of the ovaries due to damage during a cancer treatment.

The AMH analogues described herein may also be useful for reducing folliculogenesis and thus facilitate ovarian protection. Folliculogenesis is the maturation of the ovarian follicle which contains the immature oocyte, and is the progression of a number of small primordial follicles into large preovulatory follicles as part of the menstrual cycle. Depletion of the primordial follicles or primordial follicles that respond to hormonal cues, signals the beginning of menopause. By reducing folliculogenesis, the primordial follicles are preserved. Ovarian protection can reduce folliculogenesis in the female subject, or reduce the number of primordial follicles being recruited by at least 10%, at least 20%, at least 30%, at least 40%, at least 50%, at least 60%, at least 70%, at least 80%, at least 90%, at least 99%, or more as compared to in the absence of the AMH analogue.

In some examples, ovarian protection can be an inhibition of premature ovarian failure. As used herein, “premature ovarian failure” refers to the cessation of the ovarian function prior to the age of 40. Clinically, premature ovarian failure is diagnosed by high levels of follicle stimulating hormone and luteinizing hormone in the blood. Causes of premature ovarian failure include, but are not limited to, chemotherapy, radiotherapy, autoimmune disease, thyroid disease, diabetes, and surgically induced menopause (e.g., hysterectomy, or oophorectomy). Ovarian protection may also encompass methods of protecting the female reproductive system from cancer therapy regimens such as chemotherapy and radiotherapy or other artificial insults such as cytotoxic factors, hormone deprivation, growth factor deprivation, cytokine deprivation, cell receptor antibodies, and the like. Other insults include surgical insults wherein a woman's reproductive system, in part or in whole, is surgically removed. In particular, hormonal imbalance, resulting from the removal of one ovary, is fully or partially restored by administration of the AMH analogue described herein.

The AMH analogues described herein are also useful for uterine protection. As used herein, “uterine protection” refers to the protection against deleterious or adverse effects on the uterus as a result of a therapeutic agent or treatment, e.g. a chemotherapeutic agent or cytotoxic agent. “Uterine protection” refers to protecting the function of the uterus (e.g., embryo implantation, development of placenta, and capacity to carry a pregnancy to term (e.g., without miscarriage or premature birth)), and the histology of the uterus (e.g., uterine lining health). “Uterine protection” can also refer to the protection against any insult to the ovaries (e.g., surgery, e.g., caesarean section, or an injury). Uterine protection maintains at least 99%, at least 95%, at least 90%, at least 80%, at least 70%, at least 60%, at least 50%, at least 40%, at least 30%, at least 20%, at least 10% of the uterine function following administration of a therapeutic agent of treatment, as compared to the uterus function prior to said administration. Uterine protection can be an increase in the uterine lining. A thin uterine lining can lead to a hinder the capacity for an embryo to implant into the uterine lining. Non-invasive imaging, e.g., pelvic ultrasound or sonogram, can be used to assess the thickness of the uterine lining in a subject. During menstruation, the lining of the uterus is 2-4 mm; less than 2 mm indicates a thin uterine lining.

Uterine protection can inhibit or reduce the likelihood of endometriosis in a female subject. Endometriosis is condition that results in the uterine lining growing outside of the uterus, e.g., on the reproductive organs (e.g., ovaries, fallopian tubes, or the tissue surrounding the uterus). Endometriosis hinder the function of the ovaries, fallopian tubes, or the uterus, and can result in infertility. Uterine protection can reduce the incidence, or risk of, pregnancy-induced hypertension or preeclampsia.

The AMH analogues described herein may be used for contraception, meaning it halts the ability of decreases the likelihood of conception and thus pregnancy. Administration of an AMH analogue to a female subject allows said female to control menstrual cycling, and reproductive hormone secretion, and slows down, or prevents primordial follicle recruitment and/or activation (e.g., administration of AMH analogue stops menstrual cycling).

The AMH analogues described herein may be administered to prevent a decline in the functional ovarian reserve (FOR), or to reduce folliculogenesis in a female subject. The subject can between the ages of 15 and 55 years of age and will, or is being treated with, a treatment selected from immunotherapy, cell therapy, chemotherapy, radiotherapy or chemo-radiotherapy. The subject can have cancer or an autoimmune disease. The subject will, or is undergoing treatment with a cytotoxic drug. Reducing folliculogenesis in the female subject can be a reduction in the number of primordial follicles being recruited by at least 10% as compared to in the absence of the AMH analogue, or a reduction in the number of primordial follicles being recruited by between 10% and 99%, or a complete arrest in folliculogenesis as compared to in the absence of the AMH analogue.

The AMH analogue described herein may be useful for treating cancer, for example a cancer expressing the AMH receptor or an AMHR2 responsive cancer. In some embodiments, the cancer expresses AMHR2. In some embodiments, the cancer is an AMHR2 responsive cancer. In some embodiments, the AMH analogue described herein may be useful for treatment of gynaecological cancer. As used herein, “gynaecological cancer” refers to a cancer of the female reproductive system, for example cancer of the cervix, fallopian tubes, ovary, placenta, uterus, endometrium, vagina and vulva. In some embodiments the gynaecological cancer is selected from the group consisting of uterine cancer, ovarian cancer and cervical cancer. In some embodiments the gynaecological cancer is ovarian cancer or comprises an ovarian cancer cell. In some embodiments the gynaecological cancer is endometrial cancer or comprises an endometrial cancer cell. In some embodiments, the subject will be, or is being treated with, an additional agent or cancer therapy. In some examples, the subject will be, or is being treated with, a treatment selected from chemotherapy, radiotherapy, immunotherapy, cell therapy or chemo-radiotherapy. In some examples, the subject will be, or is undergoing treatment with a cytotoxic drug. In some embodiments, the expression of AMH receptor (e.g., AMHR2) is measured in a biological sample obtained from the subject, e.g., a cancer or tumour tissue sample or a cancer cell or tumour cell, e.g., a biopsy tissue sample.

### Administration of AMH Analogues

A therapeutically effective amount or dosage of the AMH analogues or polynucleotides or compositions described herein is administered to, for example, arrest folliculogenesis. For example, an effective amount is the amount of AMH analogue or nucleic acid encoding the same or nucleic acid encoding a precursor thereof to reduce the number of primordial follicles being recruited by at least 10%, at least 20%, at least 30%, at least 40%, at least 50%, at least 60%, at least 70%, at least 80%, at least 90%, or at least 99% compared to when the composition is not administered. An amount of the composition comprising an AMH analogue or nucleic acid encoding the same or nucleic acid encoding a precursor thereof administered to a female subject is considered effective when the amount is sufficient to reduce the number of primordial follicles being recruited to a desirable number, or decrease the probability of a primordial follicle being recruited to a desirable value. In some examples, the amount of composition administered is sufficient to achieve contraception.

Compositions described herein may be administered at one time or divided into sub-doses.

In some examples, administration can be chronic, e.g., one or more doses and/or treatments daily over a period of weeks or months. Examples of dosing and/or treatment schedules are administration daily, twice daily, three times daily or four or more times daily over a period of 1 week, 2 weeks, 3 weeks, 4 weeks, 1 month, 2 months, 3 months, 4 months, 5 months, or 6 months, or more. The dosage should not be so large as to cause adverse side effects.

Short or long-term administration to a subject is contemplated by the disclosure. An example of a short term administration is the administration to protect ovaries from radiation or chemical insults, or cancer treatment as described herein. In short term administration, the composition is administered, at least once, in a period of from about thirty days prior to immediately prior to exposure to the insult.

A lower dosage of the AMH analogue may be required in a more prolonged and continuous administration.

In some example, administration to the subject may be in vivo. In vivo administration encompasses orally, intravascularly, intraperitoneally, intrauterine, intra-ovarian, subcutaneously, intramuscularly, rectally, topically (powders, ointments, drops, bucally, sublingually), intravaginally, intracisternally or a combination thereof. In embodiments were intra-ovarian administration is desired, intra-ovarian administration can be achieved by several methods, including, for example, by direct injection into the ovary.

The AMH analogue thereof described herein, can be administered by any route known in the art or described herein, for example, oral, parenteral (e.g., intravenously or intramuscularly), intraperitoneal, rectal, cutaneous, nasal, vaginal, inhalant, skin (patch), or ocular. The AMH analogue may be administered in any dose or dosing regimen.

In some examples, the administration may be sufficient to maintain the ovary in a quiescent state.

In one example, a composition comprising an AMH analogue described herein can be administered to a subject for about 2, or about 3, or about 4, or about five weeks, or more than five weeks, e.g., about 2, or about 3, or about 4, or about 5, or about 6 or about 7 or more months, and then subsequently administered after an appropriate interval for an additional period of time, for example, for about 2, or about 3, or about 4, or about five days, or more than five days. Cycles of treatment may occur in immediate succession or with an interval of no treatment between cycles. Typically, where the subject is administered a composition comprising an AMH analogue as described herein for the preservation of fertility (e.g., in a method to arrest folliculogenesis), a subject can be administered the composition for a period of between about 3-4 months, or a period of between about 4-6 months, or a period of between about 6-8 months, or a period of between about 8-12 months, or a period of between about 12-24 months, or a period of between about 24-36 months or more than about 36 months, followed by an interval of no delivery.

In some examples, where the subject is administered a composition comprising an AMH analogue as described herein in a method for contraception, a subject can be administered the composition for a period of between about 3-4 months, or a period of between about 4-6 months, or a period of between about 6-8 months, or a period of between about 8-12 months, or a period of between about 12-24 months, or a period of between about 24-36 months or more than about 36 months, or for as long as the subject desires not to become pregnant, followed by an interval of no delivery.

In certain examples, a composition comprising an AMH analogue as described herein can be administered by most any means, but in some embodiments are delivered to the subject as an injection (e.g. intravenous, subcutaneous, intraarterial), infusion or instillation. In certain embodiments, the composition comprising an AMH analogue is delivered to the subject by oral ingestion or intravaginal administration.

In some examples, a AMH analogue as disclosed herein can be administered vaginally, e.g., using including hydrogels, vaginal tablets, pessaries/suppositories, particulate systems, and intravaginal rings, as known to one of ordinary skill in the art.

In some embodiments, it is preferable that the AMH analogue is administered to the subject before a chemotherapeutic treatment, immunotherapy, cytotoxic therapeutic, surgery, or radiation treatment. For example, where the AMH analogue is administered ovarian and/or uterine protection, for reducing folliculogenesis, for inhibiting premature ovarian failure, for contraception, or prevent a decline in the functional ovarian reserve (FOR) is preferable that the AMH analogue is administered to the subject before a chemotherapeutic treatment, immunotherapy, cytotoxic therapeutic, surgery, or radiation treatment.

In some examples, female subjects can be administered the following doses of AMH analogue: females 13-45 years: 1 to 10 ng/mL; females older than 45 years: Less than 1 ng/mL. Dosage values may vary depending upon, for example, the female's functional ovarian reserve or severity of the ovarian ageing or diminished ovarian reserve to be alleviated.

The efficacy and toxicity of the AMH analogue can be determined by standard pharmaceutical procedures in cell cultures or experimental animals e.g. ED₅₀ (wherein the dose is effective in 50% of the population) and LD₅₀ (the dose is lethal to 50% of the population). The dose ratio of toxic to therapeutic effects is the therapeutic index, and it can be expressed as the ratio, LD₅₀/ED₅₀.

An appropriate experimental model which can be used includes determining a dose that can be of use is the mullerian duct regression bioassay or a in vivo cancer model which is commonly known by ordinary skill in the art. In vivo cancer models are discussed in Frese et al., “Maximizing mouse cancer models” Nat Rev Cancer. 2007 September; 7(9):645-58 and Santos et al., Genetically modified mouse models in cancer studies. Clin Transl Oncol. 2008 December; 10(12):794-803, and “Cancer stem cells in mouse models of cancer”, 6th Annual MDI Stem Cell Symposium, MDI Biological Lab, Salisbury Cove, Me., Aug. 10-11, 2007″ which are incorporated herein in their entirety by reference. For example, the therapeutically effective amount of a recombinant human AMH polypeptide can be assessed in a mouse model of fertility.

If necessary, the compositions of the disclosure may be administered locally to the area in need of treatment (e.g. the ovary). This may be achieved for example by local infusion during surgery, topical application, e.g., by injection, by means of a catheter, or by means of an implant, the implant being of a porous, non-porous, or gelatinous material, including membranes, such as sialastic membranes, fibers, or commercial skin substitutes.

Agents, e.g., nucleic acid agents which encode an AMH analogue can also be delivered using a vector, e.g., a viral vector by methods which are well known to those skilled in the art.

In some examples, the AMH analogue is administered as a monotherapy. In some examples, the AMH analogue is administered in combination with (e.g., before, during and/or after) at least one additional therapeutic agent (i.e., co-administration). For example, the AMH analogue can be administered in combination with an chemotherapeutic agent, an anti-tumour agent, radiation, or surgery. The term “co-administer” indicates that each of the at least two compounds (one being the AMH analogue) are administered during a time frame wherein the respective periods of biological activity or effects overlap. Thus, the term includes sequential as well as coextensive administration of compounds. Similar to administering compounds, co-administration of more than one substance can be for therapeutic and/or prophylactic purposes. If more than one substance or compound is co-administered, the routes of administration of the two or more substances need not be the same. The scope of the methods and uses described herein are not limited by the identity of the substance or substances which may be co-administered with the AMH analogue.

In some examples, the AMH analogue is administered in combination with a checkpoint inhibitor. A checkpoint inhibitor can be a small molecule, inhibitory RNA/RNAi molecule (both single and double stranded), an antibody, antibody reagent, or an antigen-binding fragment thereof that specifically binds to at least one immune checkpoint protein. Common checkpoints that are targeted for therapeutics include, but are not limited to PD-1, CTLA4, TIM3, LAG3 and PD-LI.

In some examples, the AMH analogue is administered in combination with a immunotherapy (e.g., a drug or agent used to treat an auto-immune disease).

### Compositions

Compositions comprising an AMH analogue as described herein can be formulated in any suitable means e.g. as a sterile injectable solution containing any compatible carrier, such as various vehicles, adjuvants, additives, and diluents. Compounds utilized in the present disclosure can be administered parenterally to the subject in the form of slow-release subcutaneous implants or targeted delivery systems such as monoclonal antibodies, vectored delivery, iontophoretic, polymer matrices, liposomes, and microspheres.

Non-aqueous vehicles such a cottonseed oil, sesame oil, olive oil, soybean oil, corn oil, sunflower oil, or peanut oil and esters, such as isopropyl myristate, may also be used as solvent systems for compositions. Additionally, various additives which enhance the stability, sterility, and isotonicity of the compositions, including antimicrobial preservatives, antioxidants, chelating agents, and buffers, can be added. Prevention of the action of microorganisms can be ensured by various antibacterial and antifungal agents, e.g., parabens, chlorobutanol, phenol and sorbic acid. In many cases, it will be desirable to include isotonic agents, for example, sugars, sodium chlonde, and the like. Prolonged absorption of the injectable pharmaceutical form can be brought about by the use of agents delaying absorption, for example, aluminum monostearate and gelatin.

In some examples, the compositions may be lipid-based formulations. Examples include multivesicular liposomes, multilamellar liposomes and unilamellar liposomes which can provide a sustained release rate of the composition.

In some examples the composition used in the methods described herein can be in a controlled release form. A variety of known controlled- or extended-release dosage forms, formulations, and devices can be adapted for use with the compositions of the disclosure. Examples include those described in U.S. Pat. Nos. 3,845,770; 3,916,899; 3,536,809; 3,598,123; 4,008,719; 5674,533; 5,059,595; 5,591,767; 5,120,548; 5,073,543; 5,639,476; 5,354,556; 5,733,566; and 6,365,185. These dosage forms can be used to provide slow or controlled-release of one or more active ingredients using, for example, hydroxypropylmethyl cellulose, other polymer matrices, gels, permeable membranes, osmotic systems (such as OROS® (Alza Corporation, Mountain View, Calif. USA)), or a combination thereof to provide the desired release profile in varying proportions.

In one example, the composition is delivered in a liposome (see Langer (1990) Science 249: 1527-1533).

In some examples, a composition comprising an AMH analogue as described herein can be administered and/or formulated in conjunction (e.g., in combination) with any other therapeutic agent.

Pharmaceutical compositions of the present disclosure comprise a compound of this disclosure and a pharmaceutically acceptable carrier, wherein the compound is present in the composition in an amount which is effective to treat the condition of interest. Appropriate concentrations and dosages can be readily determined by one skilled in the art.

Pharmaceutically acceptable carriers are familiar to those skilled in the art. For compositions formulated as liquid solutions, acceptable carriers include saline and sterile water, and may optionally include antioxidants, buffers, bacteriostats and other common additives. Some examples of materials which can serve as pharmaceutically acceptable carriers include, without limitation: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations. The compositions can also be formulated as pills, capsules, granules, or tablets which contain, in addition to a compound of this invention, diluents, dispersing and surface active agents, binders, and lubricants. One skilled in this art may further formulate the compounds of this disclosure in an appropriate manner, and in accordance with accepted practices, such as those disclosed in Remington's Pharmaceutical Sciences, Gennaro, Ed., Mack Publishing Co., Easton, Pa. 1990.

In some examples, wetting agents, emulsifiers and lubricants, such as sodium lauryl sulfate and magnesium stearate, as well as colouring agents, release agents, coating agents, sweetening, flavoring and perfuming agents, preservatives and antioxidants can also be present in the compositions. Examples of pharmaceutically acceptable antioxidants include: water soluble antioxidants, such as ascorbic acid, cysteine hydrochloride, sodium bisulfate, sodium metabisulfate, sodium sulfite and the like; oil-soluble antioxidants, such as ascorbyl palmitate, butylated hydroxyanisole (BHA), butylated hydroxytoluene (BHT), lecithin, propyl gallate, alpha-tocopherol, and the like; and metal chelating agents, such as citric acid, ethylenediamine tetraacetic acid (EDTA), sorbitol, tartaric acid, phosphoric acid, and the like.

“Sustained-release”, “controlled-release”, or similar terms refer to formulations that allow the active ingredient (e.g., AMH analogue) to be released over time, and are used to maintain a more consistent level of the active ingredient in the body (e.g., in the bloodstream), and are known in the art.

Formulations of the present disclosure include those suitable for intravenous, oral, nasal, topical, transdermal, buccal, sublingual, rectal, vaginal and/or parenteral administration.

Formulations of the invention suitable for oral administration include capsules, cachets, pills, tablets, lozenges (using a flavoured basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present disclosure as an active ingredient.

Liquid dosage forms for oral administration of the compounds of the disclosure include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof Besides inert diluents, the oral compositions can also include adjuvants such as wetting agents, emulsifying and suspending agents, sweetening, flavouring, colouring, perfuming and preservative agents.

The composition may be formulated for rectal or vaginal administration, for example as a suppository, which may be prepared by mixing one or more compounds (e.g. AMH analogue) of the disclosure with one or more suitable excipients or carriers comprising, for example, cocoa butter, polyethylene glycol, a suppository wax or a salicylate, and which is solid at room temperature, but liquid at body temperature and, therefore release the active compound. Suitable carriers and formulations for such administration are known in the art.

Dosage forms for the topical or transdermal administration of a recombinant human AMH polypeptide as described herein, e.g., for muscular administration include powders, sprays, ointments, pastes, creams, lotions, gels, solutions, patches and inhalants.

Transdermal patches can be made by dissolving or dispersing the compound in the proper medium. Absorption enhancers can also be used to increase the flux of the compound across the skin. The rate of such flux can be controlled by either providing a rate controlling membrane or dispersing the active compound in a polymer matrix or gel.

Pharmaceutical compositions suitable for parenteral administration comprise one or more compounds of the disclosure in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

In certain examples, the pharmaceutical composition may optionally further comprise one or more additional therapeutic agents. Of course, such therapeutic agents are which are known to those of ordinary skill in the art can readily be identified by one of ordinary skill in the art.

In certain examples, the composition may comprise the AMH analogue conjugated or covalently attached to a targeting agent for example, to increase tissue specificity and targeting to a cell, for example muscle cells. Targeting agents can include, for example without limitation, antibodies, cytokines and receptor ligands.

### Subjects in Need of Treatment

In one example, the subject being administered an AMH analogue described herein has cancer and will be treated with, or is currently being treated with, or has been treated with a chemotherapy or anti-cancer agent. Cancer includes, for example, colon carcinoma, pancreatic cancer, breast cancer, ovarian cancer, fibrosarcoma, myxosarcoma, liposarcoma, chondrosarcoma, osteogenic sarcoma, chondroma, angiosarcoma, endotheliosarcoma, lymphangiosarcoma, lymphangioendotheliosarcoma, synovioma, mesothelioma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma, acute lymphocytic leukemia and acute myelocytic leukemia, chronic leukemia and polycythemia vera, lymphoma (Hodgkin's disease and non-Hodgkin's disease), multiple myeloma, Waldenstrom's macroglobulinemia, or immunoglobulin heavy chain diseases. The cancer may be a primary cancer or a metastatic cancer.

As used herein, an “anti-cancer agent” can refer to any therapeutic that has an intended use for the treatment of cancer (e.g., an immune checkpoint inhibitor, CAR T cells, or targeted therapies) that has been shown to have adverse effects on the uterus and/or ovaries. Damage to the ovaries and/or uterus can be measured by, e.g., the presence of cell death, tissue defects or decay, or abnormal function of the ovary or uterus (e.g., abnormal hormone secretion, increased folliculogenesis, or desensitization of follicle to hormone stimulation).

In some examples, the methods described herein enable the female subject to retain the ability and/or ovary reserves to produce viable offspring. In some examples, the administration of the composition is terminated prior to exposure of the female subject to the cytotoxic agent or chemotherapy agent, or alternatively concomitant with the treatment and/or subsequent to the treatment with the cytotoxic agent or chemotherapy agent, or cancer treatment.

In another example, the subject being administered an AMH analogue described herein has autoimmune disease and will be treated with, or is currently being treated with, or has been treated with an immunotherapy. As used herein, an “autoimmune disease or disorder” is characterized by the inability of one's immune system to distinguish between a foreign cell and a healthy cell. Non-limiting examples of autoimmune disease include oophoritis, inflammatory arthritis, type 1 diabetes mellitus, multiples sclerosis, psoriasis, inflammatory bowel diseases, SLE, and vasculitis, allergic inflammation, such as allergic asthma, atopic dermatitis, and contact hypersensitivity, rheumatoid arthritis, multiple sclerosis (MS), systemic lupus erythematosus, Graves' disease (overactive thyroid), Hashimoto's thyroiditis (underactive thyroid), chronic graft v. host disease, hemophilia with antibodies to coagulation factors, celiac disease, Crohn's disease and ulcerative colitis, Guillain-Barre syndrome, primary biliary sclerosis/cirrhosis, sclerosing cholangitis, autoimmune hepatitis, Raynaud's phenomenon, scleroderma, Sjogren's syndrome, Goodpasture's syndrome, Wegener's granulomatosis, polymyalgia rheumatica, temporal arteritis/giant cell arteritis, chronic fatigue syndrome CFS), psoriasis, autoimmune Addison's Disease, ankylosing spondylitis, Acute disseminated encephalomyelitis, antiphospholipid antibody syndrome, aplastic anemia, idiopathic thrombocytopenic purpura, Myasthenia gravis, opsoclonus myoclonus syndrome, optic neuritis, Ord's thyroiditis, pemphigus, pernicious anaemia, polyarthritis in dogs, Reiter's syndrome, Takayasu's arteritis, warm autoimmune hemolytic anemia, Wegener's granulomatosis and fibromyalgia (FM).

In one example, the female subject will be treated with, or is currently being treated with, or has been treated with, a cytotoxic drug or cytotoxic agent that causes cell death or cell damage to cells in the uterus or ovary.

In one example, the female subject will be treated with, or is currently being treated with, or has been treated with a long-term therapeutic regime, i.e., treatment for a chronic condition, relapse in chronic condition, human immunodeficiency virus (HIV), viral hepatitis, viral or bacterial meningitis, malaria, or a neurodegenerative disease. In one example, the female subject will be treated with, or is currently being treated with, or has been treated with a long-term therapeutic regime that does not result in damage to the uterus and/or ovaries. For example, a subject who is undergoing treatment for human immunodeficiency virus (HIV) may wish to delay or slow the recruitment and/or activation of primordial follicle recruitment, or preserve their fertility. The subject may wish to preserve her fertility and/or prevent pregnancy during a long-term treatment for reasons including, but not limiting to, because the therapeutic being administered has adverse effects on a foetus, or a pregnancy would not be ideal during the treatment due to side effects of the treatment (e.g., fatigue, or nausea).

In some examples, the female subject may wish to preserve their fertility for reasons other than ovarian or uterine protection during treatment. A female subject may wish to delay having children due to a number of different lifestyle factors. In some examples, a female subject is administered a AMH analogue described herein to preserve fertility, and is not undergoing additional treatment or receiving additional therapeutics. As used herein, “fertility preservation” refers to maintaining the fertility potential (the likelihood of conceiving a child based as factors e.g., age of eggs, regularity of menstrual cycle, ovarian reserve, ovarian function, ovarian hormone secretion) a subject in its existing state, e.g., at the time in which an AMH analogue is first administered to a patient. “Fertility preservation” can refer to extended fertility beyond its natural limit, e.g., past child-bearing age. “Fertility preservation” can refer to maintaining the same number of primordial follicles present in the ovary of a subject prior to administration of AMH analogue. AMH analogue can be administered to a female subject to inhibit age-related fertility decline. Age-related fertility decline” refers to a decrease in likelihood of conceiving due to the age of the female. The peak biological age for a female to have a child is in the late teens and early twenties; the rate of infertility increases with the age of the female.

In some examples, a female subject may wish to delay or slow the recruitment of primordial follicle recruitment, or preserve their fertility, if the subject has, or is pre-disposed diminished ovarian reserve (DOR), premature ovarian aging (POA), primary ovarian insufficiency (POI), endometriosis, one or more FMR1 premutations or 55-200 GCC FMR1 repeats, BRAC1 mutations, Turner syndrome, an autoimmune disease, an ovarian autoimmune disease (e.g., oophoritis) thyroid autoimmunity, adrenal autoimmunity or autoimmunity polyglandular syndromes.

In some examples, the AMH analogues are used as a contraceptive in a subject. Accordingly, the present disclosure also provides a method of contraception comprising administering to a subject an AMH analogue as described herein. In one example, the subject is undergoing chemotherapy. In a further example, the contraceptive comprises a polynucleotide encoding an AMH analogue as described herein within an adenovirus vector.

### Kits

The disclosure also provides a kit for use in any method described herein, the kit comprising a pump or infusion device comprising a recombinant AMH polypeptide described herein and instructions for implanting the pump or infusion device into the female subject for the treatment of a subject.

In one example, the female subject requiring treatment has one or more of a diminished ovarian reserve (DOR), premature ovarian aging (POA), primary ovarian insufficiency (P01), endometriosis, one or more FMR1 premutations or 55-200 GCC FMR1 repeats, or where the subject is undergoing, has, or will undergo a cancer treatment.

A kit is any manufacture (e.g., a package or container) comprising at least one reagent, e.g., AMH analogue, the manufacture being promoted, distributed, or sold as a unit for performing the methods described herein.

The kits described herein can optionally comprise additional components useful for performing the methods described herein. By way of example, the kit can comprise fluids (e.g., buffers) suitable for composition comprising an AMH analogue as described herein, an instructional material which describes performance of a method as described herein, and the like. A kit can further comprise devices and/or reagents for delivery of the composition as described herein. Additionally, the kit may comprise an instruction leaflet and/or may provide information as to the relevance of the obtained results.

It will be appreciated by persons skilled in the art that numerous variations and/or modifications may be made to the above-described embodiments, without departing from the broad general scope of the present disclosure. The present embodiments are, therefore, to be considered in all respects as illustrative and not restrictive.

### EXAMPLES

### Methods

AMH cDNA Sources and Site-Directed Mutagenesis

A cDNA sequence encoding a modified form of full-length human anti-Müllerian hormone (hAMH) contained within the mammalian expression vector pcDNA3.1 was kindly provided by Dr Axel Themmen (Erasmus University, The Netherlands), and has been described previously by his laboratory (Weenen C et al. (2004) Molecular Human Reproduction 10(2):77-83). In brief, modifications to the hAMH cDNA were the insertion of a His-6 purification tag after Glu29. Additionally, the proteolytic processing site at the 3′ end of the prodomain had been substituted to ‘RARR’ to enhance protein maturation (referred to herein as plasmid hAMH).

Two additional modifications to hAMH were made by overlap-extension PCR, with the modified cDNA then subcloned into pCDNA3.1(—) (Thermo Fisher Scientific, Waltham, Mass.) between the restriction sites NotI and HindIII. The first modification was to further enhance protein maturation by modifying the proteolytic processing site to the theoretically ideal ‘ISSRKKRSVSS’ Super-cut motif (Duckert, Brunak & Blom (2004) Protein Engineering Design & Selection 17(1):107-112). The Super-cut cDNA sequence was inserted between the sequence for Gly447 and Ser452, and in place of the ‘RARR’ residues (referred to herein as plasmid hAMH+SCUT). Replacement of the hAMH leader sequence (the first 25 amino acids) with a human serum albumin leader sequence has previously been reported to enhance the recombinant production of hAMH from a mammalian cell line (Peepin D et al. (2013) Technology 1(1):63-71). Therefore, the hAMH+SCUT cDNA was further modified to replace the amino acids N-terminal of the His-6 tag (residues MRDLPLTSLA LVLSALGALL GTEALRAEE) with a rat serum albumin (RSA) signal peptide sequence ‘MKWVTFLLLLFISGSAFS’ (referred to herein as hAMH+SCUT+RSA). The hAMH+SCUT+RSA plasmid was subsequently used as a template for further modifications, which were carried out using the QuikChange Lightning Site-Directed Mutagenesis Kit (Agilent Technologies, Santa Clara, Calif.) according to the manufacturer's instructions. For each construct generated, the entire cDNA cassette was confirmed by DNA sequencing carried out by Micromon genomics facility (Monash University, Clayton, VIC).

### Expression and Purification of Recombinant AMH Proteins

Production of recombinant AMH proteins was by transient transfection of HEK-293T cells using polyethylenimine (PEI)-MAX (Linear; MW 40,000) (Polysciences, Warrington, Pa.). For small scale production to assess the molecular forms being secreted, cells were plated at 8×10⁵ cells/well in 6-well plates coated with poly-D-lysine (Sigma-Aldrich) in Dulbecco's modified Eagle medium (DMEM) supplemented with 10% fetal calf serum (FCS) and incubated at 37° C. in 5% CO₂. After overnight incubation, the medium was changed to OPTI-MEM (Life Technologies, Carlsbad, Calif.) medium. Plasmid DNA (5 μg/well) was then combined with PEI-MAX for 10 minutes. DNA-PEI complexes were added directly to cells and incubated in OPTI-MEM medium for 4 hours at 37° C. in 5% CO₂ before replacing with fresh OPTI-MEM medium and incubating a further 90 hours before collection.

Following secretion, the AMH prodomain and mature domain remain non-covalently associated as a pro-mature complex (Pepinsky et al. (1988) Journal of Biological Chemistry 263(35):18961-18964). Therefore the purification strategy targeted the poly-histidine tag located on the N-terminus of the prodomain. This approach allowed the mature domain, which is responsible for the hormones bioactivity, to be purified without tagging. Prior to purification, larger scale production (200 mL) was first carried out using similar methodology to that described above. In brief, 10×10⁶ cells/plate were seeded in DMEM supplemented with 10% FCS onto 15 cm plates coated with poly-D-lysine and incubated at 37° C. in 5% CO₂. After overnight incubation, the medium was changed to OPTI-MEM medium. Plasmid DNA (60 μg DNA/plate) was combined with PEI-MAX for 10 minutes. DNA-PEI complexes were added directly to cells and incubated in OPTI-MEM medium for 4 hours at 37° C. in 5% CO₂ before replacing with fresh OPTI-MEM medium containing 0.02% bovine serum albumin (BSA) and incubated for 90 hours before collection.

Conditioned media containing recombinant proteins was pooled and concentrated (twice to ensure effective buffer exchange) by centrifugation (Centricon Plus-70, 5 kDa MW cut-off; Millipore, Billerica, Mass.) to ˜1 mL, then resuspended in binding buffer [50 mM phosphate buffer, 300 mM NaCl, pH 7.4] to a final volume of 5 mL. The concentrated media was subjected to cobalt-based immobilized metal affinity chromatography (Co-IMAC) by rolling in a column containing ˜0.5 mL of HisPur™ Cobalt Resin (Thermo Fisher Scientific) for ˜2.5 hours at room-temperature. Before elution, the beads were washed twice with 4 mL of binding buffer. Bound proteins were eluted by rolling in 3 mL of elution buffer [50 mM phosphate buffer, 300 mM NaCl, 500 mM imidazole] for ˜2.5 hours at room-temperature. To elute any proteins remaining bound, the HisPur™ Cobalt Resin was rolled in 3 mL of 1M imidazole [50 mM phosphate buffer, 300 mM NaCl, 1M imidazole] for ˜1 hour at room-temperature. Imidazole was removed from purified proteins by dialysis using 2 mL 3.5K MW Cut-off Slide-A-Lyzer® MINI Dialysis Devices (Thermo Fisher Scientific) according to the manufacturer's guidelines. Buffer exchange was with Dulbecco's phosphate buffered saline (Life Technologies). Purified proteins were stored at −80° C. in Protein LoBind Tubes (Eppendorf, Hamburg, Germany).

### AMH Protein Analysis by Western Blotting

Western blotting was used to assess the molecular forms of AMH being secreted into conditioned media and to provide mass estimates following Co-IMAC and dialysis. In brief, conditioned media was concentrated 12.5-fold with Nanosep microconcentrators (3 kDa MW cut-off; Pall Life Sciences, Port Washington, N.Y.), whilst Co-IMAC fractions required no further concentrating. Samples were prepared with 2×NuPAGE® LDS Sample Buffer (Life Technologies)+5% 2-mercaptoethanol (Bio-Rad, Hercules, Calif.).

Prepared samples were heated for ˜3 minutes in boiled water (˜95° C.). Proteins were separated in 10% handcast sodium dodecyl sulphate-polyacrylamide gel electrophoresis (SDS-PAGE) gels run in a Mini-PROTEAN® Vertical Electrophoresis Cell (Bio-Rad) with top running buffer [3.5 mM SDS, 100 mM tricine, 100 mM Trizma® base (Sigma-Aldrich)] and bottom running buffer [pH 8.9, 200 mM Trizma® base]. The unit ran at 80 volts until the dye front reached the separating gel and formed a single even line, after which the voltage was increased to 150 volts and run until the dye front ran off the bottom of the gel. Following separation by SDS-PAGE, gels were placed against a 0.45 μM nitrocellulose membrane (Bio-Rad) and assembled together into a Mini Trans-Blot® Cell (Bio-Rad) filled with Western transfer buffer [10% methanol, 200 mM glycine, 25 mM Trizma® base]. The transfer unit ran at 100 volts for at least 1 hour. Following transfer, the membrane was placed in blocking solution [1% BSA (Sigma-Aldrich) in tris-buffered saline (TBS) [pH 7.5, 25 mM Trizma® base, 250 mM NaCl]+0.05% Tween® 20 (Sigma-Aldrich)] for at least 1 hour, before then probing overnight with primary antibody (diluted 1:5000 in blocking solution) whilst shaking. Following overnight incubation with primary antibody at room temperature, the membrane was washed three times for 5 minutes each wash with TBS-Tween® [pH 7.5, 25 mM Trizma® base, 250 mM NaCl+0.05% Tween® 20] followed by two 5 minute washes with TBS. This was followed by 1 hour incubation at room temperature with horseradish peroxidase-conjugated anti-mouse IgG (GE Healthcare, Buckinghamshire, UK) secondary antibody (diluted 1:10,000 in blocking solution). Before developing, the membrane was washed five times again as detailed above. Membrane development was by the addition of 1 mL Lumi-Light Luminol/Enhancer solution (Roche, Basel, Switzerland) followed by 1 mL Lumi-Light stable peroxide solution (Roche). Images of chemiluminescent development were captured by a ChemiDoc™ (Bio-Rad) and analysed with Image Lab™ software (Bio-Rad).

To detect mature AMH protein, membranes were probed with mAb-5/6A (Oxford Brookes University, Oxford, UK). mAb-5/6A was raised to a 32 amino acid peptide (VPTAYAGKLLISLSEERISAHHVPNMVATECG, amino acids 527-558 in hAMH) corresponding to a region towards the C-terminus of the hAMH mature domain (Weenen et al. (2004) Molecular Human Reproduction 10(2):77-83). To detect the human AMH prodomain, membranes were probed with mAb-9/6A (Oxford Brookes University). mAb-9/6A was developed by immunising mice against the entire hAMH protein. mAb-9/6A has previously been reported to specifically detect the processed hAMH prodomain and was initially selected by its developers as a detection antibody for an AMH ELISA (Al-Qahtani et al. (2005) Clinical Endocrinology 63(3):267-273).

To assess molecular weight sizes the pre-stained protein standard SeeBlue Plus2 (Life Technologies) was used. Concentration estimates were determined using a commercially available recombinant hAMH mature domain (R&D Systems, Minneapolis, Minn.) as a standard. A range of five mass standards were used alongside four different volumes of purified material and the mean concentration of the four volumes was used as an estimate of the actual concentration. In the case of AMH mutants A546M and H548K, the epitope recognised by mAb-5/6A was disrupted. Therefore, detection and subsequent concentration estimates were determined by probing the membrane with mAb-9/6A and assessing densitometry of the processed prodomain relative to a previously quantitated preparation of pro-mature AMH.

In Vitro Transcriptional Reporter Assay with COV434 Cells

Granulosa cells are an AMH target within the ovary (Pepin, Sabatini & Donahoe (2018) Current Opinion in Endocrinology Diabetes and Obesity 25(6):399-405), with AMH activity mediated intracellularly via Smad-1/5 signalling (Sedes L et al. (2013) PLoS One 8(11):13). COV434 cells are an immortalised human granulosa cell line (Zhang H et al. (2000) Molecular Human Reproduction 6(2):146-153 previously reported not to endogenously express AMH (Weenen C et al. (2004) Molecular Human Reproduction 10(2):77-83), allowing them to be used as a human granulosa cell model for assessing AMH activity without interference from endogenous AMH. This laboratory (Al-Musawi S L et al. (2013) Endocrinology 154(2):888-899; Patino L C et al. (2017) Journal of Clinical Endocrinology & metabolism 102(3):1009-1019) and others (Moore, Otsuka & Shimasaki (2003) Journal of Biological Chemistry 278(1):304-310; Peng J et al. (2013) PNAS 110(8):E776-E785) have previously reported COV434 cells to show a robust Smad-1/5 response following treatment with BMP ligands. Therefore, to assess activation of the Smad-1/5 pathway by the Co-IMAC purified AMH proteins generated during this study, the Smad1/5-responsive BRE-luciferase assay was used in COV434 cells. This involved transient transfection of BRE-Luc, a plasmid containing a luciferase gene downstream of a promoter with two copies of BMP-response elements isolated from the Id1 gene promoter (Korchynskyi & ten Dijke (2002) JBC 277(7):4883-4891). Additionally, to ensure the cells were responsive to AMH, the cells were co-transfected with a small amount of plasmid for the AMH-specific type II receptor, AMHR2 (Imbeaud S et al. (1995) Nature Genetics 11(4):382-388).

In brief, cells were plated at 7.5×10⁴ cells/well in DMEM/10% FCS onto 48-well plates coated with poly-D-lysine and grown overnight at 37° C. in 5% CO₂. The following day, the cells were transfected with 250 ng/well of plasmid DNA composed of 248.44 ng of pBRE-Luc and 1.56 ng of pAMHR2 (GenScript HK Limited, Hong Kong; Catalogue No: OHu22327D; NM_020547). The plasmid DNA was combined with Lipofectamine 3000 (Life Technologies) according to the manufacturer's instructions, then added directly to the cells and incubated for 24 hours at 37° C. in 5% CO₂. The following day, transfected COV434 cells were treated with dose ranges of AMH variants diluted in low-serum media [DMEM with 50 mM HEPES and 0.2% FCS]. This occurred by removing transfection media from the cells and replacing with 200 μL treatment media per well. Cells were then incubated in treatment media overnight at 37° C. in 5% CO₂ with each dose tested in at least triplicate. To assess expression of luciferase protein, the media was removed and cells lysed in solubilisation buffer [26 mM glycylglycine (pH 7.8), 16 mM MgSO₄, 4 mM EGTA, 900 μM dithiothreitol, 1% Triton X-100] whilst shaking on ice for 20 minutes. The lysate was transferred to a white 96-well plate. Luciferase expression was assessed by measuring luminescence immediately after the addition of a mixture containing the substrate D-luciferin [25 mM glycylglycine (pH 7.8), 15 mM MgSO₄, 4 mM EGTA, 1 mM dithiothreitol, 1.5 mM ATP, 0.5 mM D-luciferin (Life Technologies)] using a CLARIOstar microplate reader (BMG Labtech, Ortenberg, Germany). Luciferase activity was analysed as the fold-change relative to baseline activity (Chand A L et al. (2007) Human Reproduction 22(12):3241-3248). Example 1 AMH Protein Sequences and Modifications

The sequence of native human AMH protein is available from GenBank as identifier AAH49194.1. This sequence is provided below.

(SEQ ID NO: 1)
MRDLPLTSLALVLSALGALLGTEALRAEEPAVGTSGLIFREDLDWPPGSP
QEPLCLVALGGDSNGSSSPLRVVGALSAYEQAFLGAVQRARWGPRDLATF
GVCNTGDRQAALPSLRRLGAWLRDPGGQRLVVLHLEEVTWEPTPSLRFQE
PPPGGAGPPELALLVLYPGPGPEVTVTRAGLPGAQSLCPSRDTRYLVLAV
DRPAGAWRGSGLALTLQPRGEDSRLSTARLQALLFGDDHRCFTRMTPALL
LLPRSEPAPLPAHGQLDTVPFPPPRPSAELEESPPSADPFLETLTRLVRA
LRVPPARASAPRLALDPDALAGFPQGLVNLSDPAALERLLDGEEPLLLLL
RPTAATTGDPAPLHDPTSAPWATALARRVAAELQAAAAELRSLPGLPPAT
APLLARLLALCPGGPGGLGDPLRALLLLKALQGLRVEWRGRDPRGPGRAQ
RSAGATAADGPCALRELSVDLRAERSVLIPETYQANNCQGVCGWPQSDRN
PRYGNHVVLLLKMQARGAALARPPCCVPTAYAGKLLISLSEERISAHHVP
NMVATECGCR

The 25 amino acid signal sequence is indicated by underlining. The proteolytic processing site is indicated in bold.

The sequence of the wild-type mouse AMH protein sequence is available from GenBank as identifier NP_031471.2. This sequence is provided below.

(SEQ ID NO: 2)
MQGPHLSPLVLLLATMGAVLQPEAVENLATNTRGLIFLEDELWPPSSPPE
PLCLVTVRGEGNTSRASLRVVGGLNSYEYAFLEAVQESRWGPQDLATFGV
CSTDSQATLPALQRLGAWLGETGEQQLLVLHLAEVIWEPELLLKFQEPPP
GGASRWEQALLVLYPGPGPQVTVTGTGLRGTQNLCPTRDTRYLVLTVDFP
AGAWSGSGLILTLQPSREGATLSIDQLQAFLFGSDSRCFTRMTPTLVVLP
PAEPSPQPAHGQLDTMPFPQPGLSLEPEALPHSADPFLETLTRLVRALRG
PLTQASNTQLALDPGALASFPQGLVNLSDPAALGRLLDWEEPLLLLLSPA
AATEREPMPLHGPASAPWAAGLQRRVAVELQAAASELRDLPGLPPTAPPL
LARLLALCPNDSRSSGDPLRALLLLKALQGLRAEWHGREGRGRTGRSAGT
GTDGPCALRELSVDLRAERSVLIPETYQANNCQGACAWPQSDRNPRYGNH
VVLLLKMQARGAALGRLPCCVPTAYAGKLLISLSEERISAHHVPNMVATE
CGCR

The signal sequence is indicated by underlining.

The human and mouse AMH sequences share 73% identity.

hAMH+SCUT+RSA Modification

The wild-type human AMH sequence was modified as described in the methods to incorporate a His6 tag, a super-cut pro-domain cleavage site and an RSA signal peptide. This protein was designated hAMH+SCUT+RSA and the protein sequence is provided below.

(SEQ ID NO: 3)

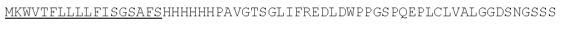

PLRVVGALSAYEQAFLGAVQRARWGPRDLATFGVCNTGDRQAALPSLRRLGAWLQDPGGQ
RLVVLHLEEVTWEPTPSLRFQEPPPGGAGPPELALLVLYPGPGPEVTVTRAGLPGAQSLCPSR
DTRYLVLAVDRPAGAWRGSGLALTLQPRGEDSRLSTARLQALLFGDDHRCFTRMTPALLLLP
RSEPAPLPAHGQLDTVPFPPPRPSAELEESPPSADPFLETLTRLVRALRVPPARASAPRLALDP
DALAGFPQGLVNLSDPAALERLLDGEEPLLLLLRPTAATTGDPAPLHDPTSAPWATALARRVA
AELQAAAAELRSLPGLPPATAPLLARLLALCPGGPGGLGDPLRALLLLKALQGLRVEWRGRDP
RGPGISSRKKRSVSSSAGATAADGPCALRELSVDLRAERSVLIPETYQANNCQGVCGWPQSD
RNPRYGNHVVLLLKMQARGAALARPPCCVPTAYAGKLLISLSEERISAHHVPNMVATECGCR

The signal sequence is indicated by underlining. The His6 tag is highlighted by shading. The proteolytic processing site is indicated in bold. Processing of hAMH+SCUT+RSA as defined in SEQ ID NO:3 produces mature AMH having the sequence provided in SEQ ID NO:36.

The corresponding nucleic acid sequence is provided below:

(SEQ ID NO: 4)
atgaagtgggtaacctttctcctcctcctcttcatctccggttctgcctt
ttcccatcatcatcatcatcatccagctgtgggcaccagtggcctcatct
tccgagaagacttggactggcctccaggcagcccacaagagcctctgtgc
ctggtggcactgggcggggacagcaatggcagcagctcccccctgcgggt
ggtgggggctctaagcgcctatgagcaggccttcctgggggctgtgcaga
gggcccgctggggcccccgagacctggccaccttcggggtctgcaacacc
ggtgacaggcaggctgccttgccctctctacggcggctgggggcctggct
gcaggaccctggggggcagcgcctggtggtcctacacctggaggaagtga
cctgggagccaacaccctcgctgaggttccaggagcccccgcctggagga
gctggccccccagagctggcgctgctggtgctgtaccctgggcctggccc
tgaggtcactgtgacgagggctgggctgccaggtgcccagagcctctgcc
cctcccgagacacccgctacctggtgttagcggtggaccgccctgcgggg
gcctggcgcggctccgggctggccttgaccctgcagccccgcggagagga
ctcccggctgagtaccgcccggctgcaggcactgctgttcggcgacgacc
accgctgcttcacacggatgaccccggccctgctcctgctgccgcggtcc
gagcccgcgccgctgcctgcgcacggccagctggacaccgtgcccttccc
gccgcccaggccatccgcggaactggaggagtcgccacccagcgcagacc
ccttcctggagacgctcacgcgcctggtgcgggcgctgcgggtccccccg
gcccgggcctccgcgccgcgcctggccctggatccggacgcgctggccgg
cttcccgcagggcctagtcaacctgtcggaccccgcggcgctggagcgcc
tactcgacggcgaggagccgctgctgctgctgctgaggcccactgcggcc
accaccggggatcctgcgcccctgcacgaccccacgtcggcgccgtgggc
cacggccctggcgcgccgcgtggctgctgaactgcaagcggcggctgccg
agctgcgaagcctcccgggtctgcctccggccacagccccgctgctggcg
cgcctgctcgcgctctgtccaggaggccccggcggcctcggcgatcccct
gcgagcgctgctgctcctgaaggcgctgcagggcctgcgcgtggagtggc
gcgggcgggatccgcgcgggccgggtatctcatcgagaaagaaacgctca
gtctcatcaagcgcgggggccaccgccgccgacgggccgtgcgcgctgcg
cgagctcagcgtagacctccgcgccgagcgctccgtactcatccccgaga
cctaccaggccaacaattgccagggcgtgtgcggctggcctcagtccgac
cgcaacccgcgctacggcaaccacgtggtgctgctgctgaagatgcaggc
ccgtggggccgccctggcgcgcccaccctgctgcgtgcccaccgcctacg
cgggcaagctgctcatcagcctgtcggaggagcgcatcagcgcgcaccac
gtgcccaacatggtggccaccgagtgtggctgccggtaa

### Example 2 Recombinant AMH Protein Expression

Modifications were made to the hAMH cDNA via overlap-extension PCR with the aim of improving precursor processing and protein secretion. To assess whether the desired outcomes were achieved, HEK-293T cells were transiently transfected with the modified expression vectors (hAMH+SCUT and hAMH+SCUT+RSA respectively) containing the modified hAMH cDNA.

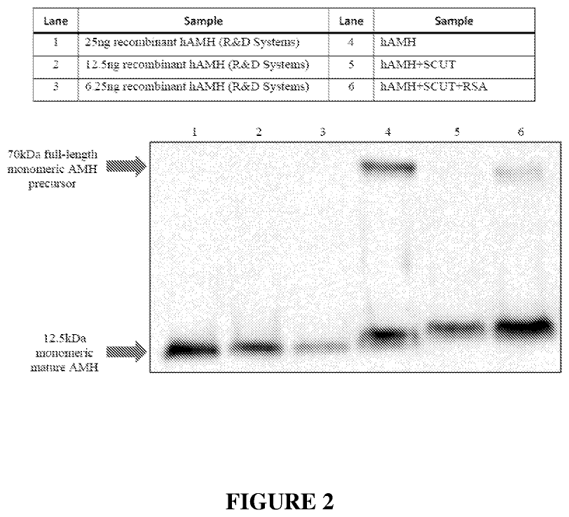

Analysis of the concentrated and reduced conditioned media by Western blotting with mAb-5/6A (FIG. 2) detected both the 12.5 kDa monomeric AMH mature domain and 70 kDa monomeric AMH precursor (FIG. 2; lane 4). The AMH variant containing the Super-Cut (SCUT) modification displayed improved precursor processing as judged qualitatively by faint detection of the 70 kDa precursor form relative to the 12.5 kDa mature form within the same sample (FIG. 2; lane 5). The hAMH form containing both the SCUT modification and RSA signal peptide (FIG. 2; lane 6) was used as the template for subsequent mutations targeted to the potential receptor-binding epitopes.

### Example 3 Mutations Targeted to the Potential Receptor-Binding Epitopes of hAMH

The structures of Transforming Growth Factor-β (TGF-β) superfamily ligands in complex with their receptors have previously been reported for a number of the superfamily members, (Hinck, Mueller & Springer (2016) Cold Spring harbor Perspectives in Biology 8(12):51. Using these reported structures and protein sequence homology as a guide, the inventors attempted to identify amino acids within hAMH which mediate the unique interaction with its type II receptor, AMHR2. To this aim, in vitro site-directed mutagenesis was used to produce a number of hAMH mutant expression vector cohorts.

Cohort One hAMH Mutant

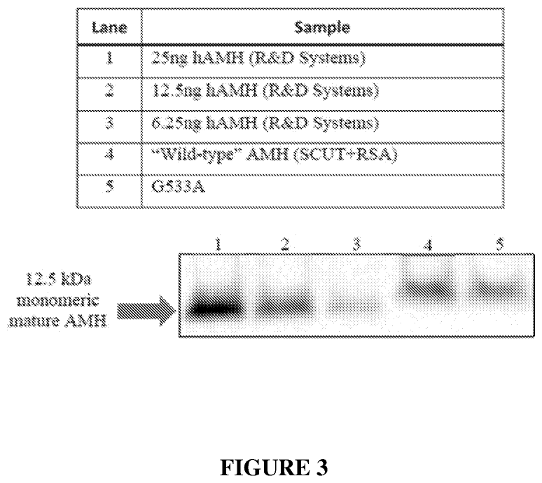

Initially (FIG. 3), the G533A and L535A mutants were generated. G533 and L535 are indicated in bold and underline below in the mature native hAMH sequence.

(SEQ ID NO: 5)
SAGATAADGPCALRELSVDLRAERSVLIPETYQANNCQGVCGWPQSDRNP
RYGNHVVLLLKMQARGAALARPPCCVPTAYAGKLLISLSEERISAHHVPN
MVATECGCR

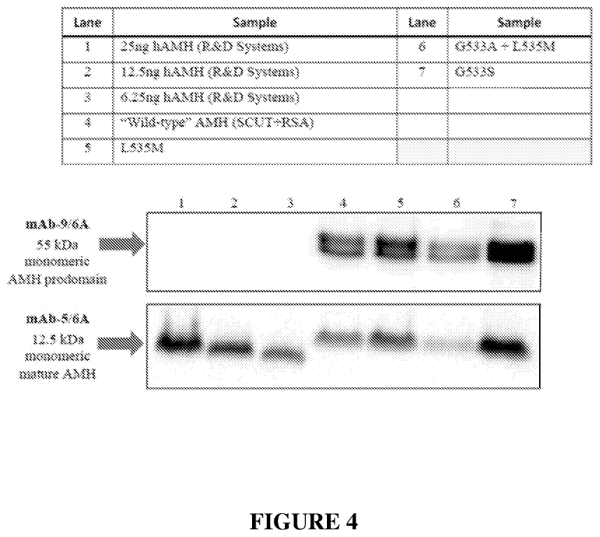

The L535A mutant was secreted poorly (data not shown). Therefore, the inventors generated the more conservative mutation, L535M, to try and assess the role of this amino acid (FIG. 4).

Cohort Two hAMH Mutants

In the second cohort (FIG. 4), the following mutants was generated: G533S and the double mutant G533A+L535M.

Cohort Three hAMH Mutants

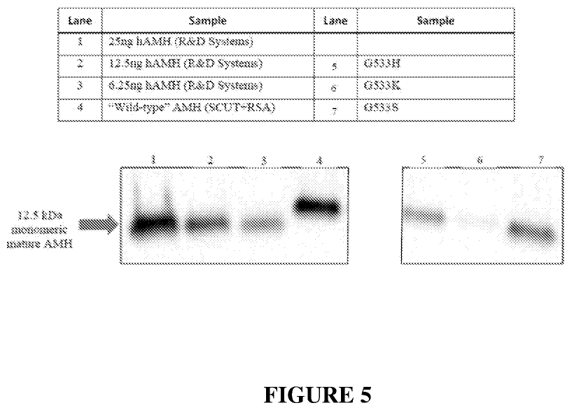

In the third cohort (FIG. 5) the following mutants were generated: G533H, G533K, G533R and G533L. The G533L mutant was not secreted, whilst the G533R mutant was poorly secreted, therefore the activity of these two mutants was not determined.

Cohort Four hAMH Mutants

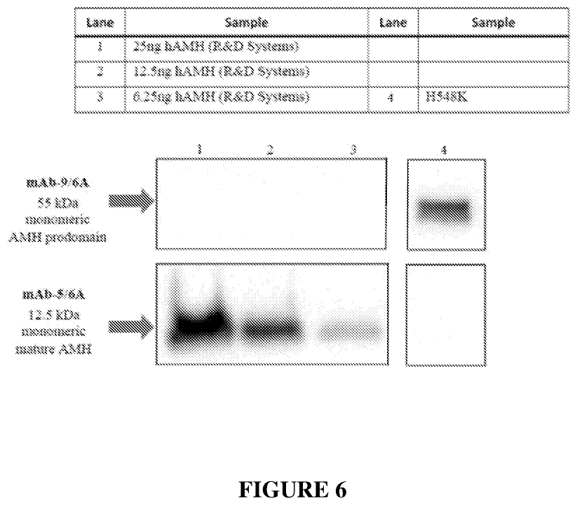

In the fourth cohort (FIG. 6) the H548K mutant was generated.

### Example 4 Purification of Recombinant AMH Protein

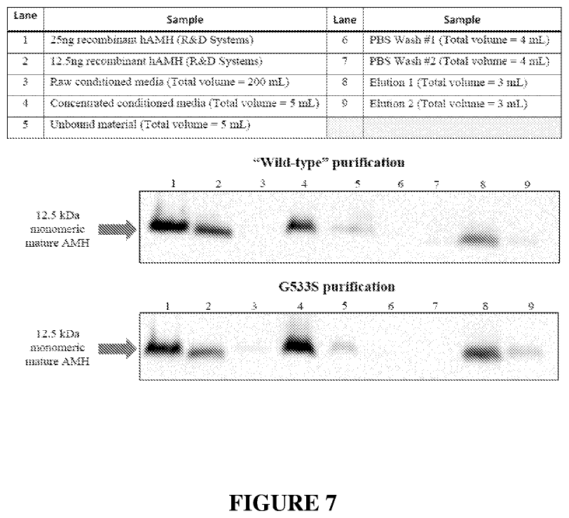

To purify AMH, HisPur™ cobalt resin was used which targeted the poly-histidine tag located on the N-terminus of the AMH prodomain. This approach allowed the mature domain, which is responsible for the hormones bioactivity, to be purified without tagging. Briefly, after concentrating 200 mL of conditioned media and incubating for ˜2.5 hours with the resin, unbound proteins were collected and the resin washed twice with PBS to remove any loosely bound proteins. Two different concentrations of imidazole were used to release the cobalt-bound AMH. Recoveries were assessed by Western blotting probed with mAb-5/6A, with the majority of bound AMH recovered in the first elution. Some AMH protein was also eluted in the second elution containing a higher concentration of imidazole, whilst some AMH protein did not bind to the resin. Imidazole was removed from the preparation by dialysis against Dulbecco's phosphate buffered saline. Mutant AMH proteins were recovered at similar proportions to hAMH+SCUT+RSA (designated as “wild-type” herein), see FIG. 7.

### Example 5 Activity of Recombinant AMH Proteins

To assess the activity of the purified recombinant AMH preparations generated, COV434 cells were transfected with the Smad1/5-responsive BRE-luciferase reporter and AMHR2. Twenty four (24) hours later, the cells were treated overnight with a dose range of AMH before lysis and measurement of luminescence immediately after the addition of the substrate D-luciferin. Luciferase activity was analysed as the fold-change related to baseline activity. The different AMH mutants have been grouped below on their ability to stimulate the BRE-luciferase reporter relative to hAMH+SCUT+RSA (Wild type) AMH.

(i) AMH Mutant Proteins with Moderate to Major Increases in Activity

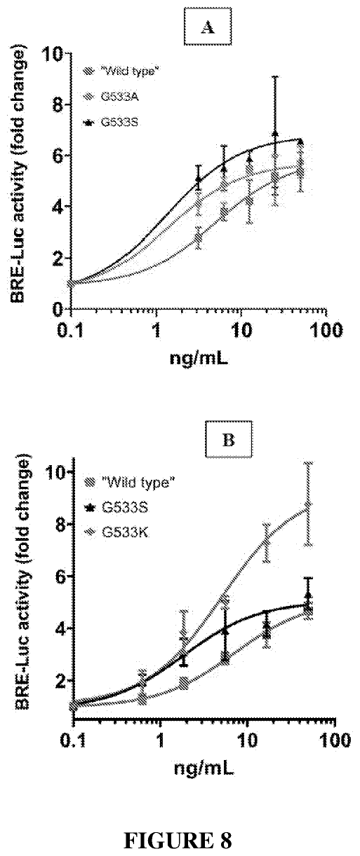

hAMH+SCUT+RSA (Wild type) AMH typically gives a peak response in the COV434 BRE-luciferase assay of between 15-50 ng/mL, with an EC50 of ˜6 ng/mL. The G533A mutant displayed ˜2-fold greater activity than “wild-type” AMH (FIG. 8A), whilst the G533S mutant displayed ˜3-fold greater activity (FIG. 8A,B). The G533K mutant displayed ˜5-fold greater activity than “wild type” AMH, whilst also stimulating a substantially higher maximal response at the top dose (FIG. 8B).

(ii) AMH Mutant Proteins with No Observable Change in Activity

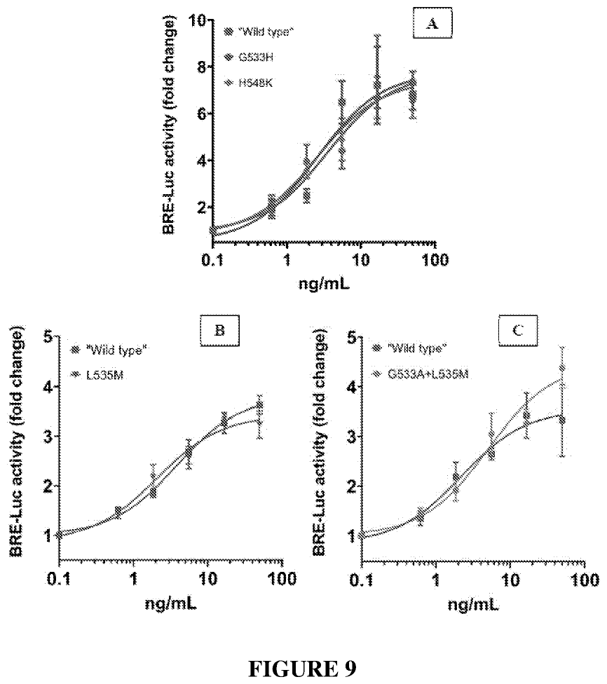

The mutants G533H, H548K, L535M and the double mutant G533A+L535M displayed no observable difference relative to hAMH+SCUT+RSA (Wild type) AMH in the lucifierase assay (FIG. 9).

The hAMH mutants generated are summarised in Table 5 below.

TABLE 5
hAMH mutants

| | Protein secreted | EC₅₀ in COV434 | | Potency relative | AMH activity relative | Maximal response relative |
|---|---|---|---|---|---|---|
| Mutation | (Yes/No) | BRE-Luc assay | | to ‘wild-type’ | to wild type (%) | to wild-type (%) |
| “WT” | Yes | ~6 | ng/mL | | 100 | |
| G533A | Yes | ~2.8 | ng/mL | ~2-fold more potent | 250 | comparable |
| G533S | Yes | ~1.8 | ng/mL | ~3-fold more potent | 350 | ~140 |
| G533K | Yes | -1.2 | ng/mL | ~5-fold more potent | 500 | ~190 |
| G533H | Yes | ~6 | ng/mL | comparable | 100 | comparable |
| G533R | Yes (poorly | ND | | ND | ND | ND |
| | produced) | | | | | |
| G533L | No | NA | | NA | NA | NA |
| L534A | Yes | | | Inactive | | Inactive |
| L535A | Yes (poorly | ND | | | ND | |
| | produced) | | | | | |
| L535M | Yes | ~6 | ng/mL | comparable | 100 | comparable |
| G533A + L535M | Yes | ~6 | ng/mL | comparable | 100 | comparable |
| L536A | Yes (poorly | ND | | ND | ND | ND |
| | produced) | | | | | |
| L536M | Yes | | | ~3-fold less potent | | ~80 |
| L536I | Yes | | | ~7-fold less potent | | ~65 |
| L536N | No | NA | | NA | NA | NA |
| S538A | Yes | | | ~4-fold less potent | | ~65 |
| L539A | Yes (poorly | ND | | ND | ND | ND |
| | produced) | | | | | |
| A546M | Yes | | | Almost inactive | | ~5 |
| H548K | Yes | ~6 | ng/mL | comparable | 100 | comparable |
| AMH-BMP2 chimera | Yes | | | Almost inactive | | |
| (Substituted Ala473-Ser476 | | | | | | |
| to the homologous portion | | | | | | |
| of BMP2 “DVGWNDW”) | | | | | | |

The location of the G533 residue in the finger domain of AMH is shown in FIG. 10.
Potency, activity and response is relative to “WT” mature AMH (produced from hAMH + SCUT + RSA; SEQ ID NO: 36). The inventors have shown that the activity of mature processed AMH produced from hAMH + SCUT + RSA (SEQ ID NO: 36) is comparable to the activity of hAMH purchased from R&D Systems.
G = glycine
A = alanine
K = lysine
S = serine
H = histidine
R = arginine
L = leucine
M = methionine
I = isoleucine
N = asparagine

1. An isolated anti-mullerian hormone (AMH) analogue, comprising a C-terminal domain sequence, wherein the C-terminal domain comprises at least one amino acid residue modification relative to a native human mature processed AMH polypeptide set forth in SEQ ID NO:5, wherein the modification is present within one or more of amino acid residues 533 to 548 of SEQ ID NO:1.

2. The AMH analogue of claim 1, wherein the AMH analogue has at least 2.5-fold greater activity compared to the activity of the native mature processed AMH polypeptide.

3. The AMH analogue of claim 1 or claim 2, further comprising a N-terminal domain comprising an amino acid sequence which has at least 90% identity to amino acid residues 30 to 447 of SEQ ID NO:1.

4. The AMH analogue of claim 3, wherein the N-terminal domain comprises a proprotein convertase site that comprises X₁X₂X₃RKKRX₈X₉X₁₀X₁₁ (SEQ ID NO:37), wherein X₁ is absent or isoleucine, X₂ is absent or serine, X₃ is absent or serine, X₈ is absent or serine, X₉ is absent or valine, X₁₀ is absent or serine and X₁₁ is absent or serine.

5. The AMH analogue of claim 4, wherein the proprotein convertase site comprises ISSRKKRSVSS (SEQ ID NO:6).

6. The AMH analogue of claim 4, wherein the proprotein convertase site comprises RKKR (SEQ ID NO:40).

7. The AMH analogue according to any one of claims 3 to 6, wherein the N-terminal domain and C-terminal domain are separate polypeptides.

8. The AMH analogue according to any one of claims 1 to 7, wherein the modification present within amino acid residues 533 to 548 of SEQ ID NO:1 is at least one amino acid substitution.

9. The AMH analogue according to claim 8, wherein the modification present within amino acid residues 533 to 548 of SEQ ID NO:1 is a single amino acid substitution.

10. The AMH analogue according to claim 8 or 9, wherein the amino acid substitution is located at amino acid residue 533, 535 or 548 of SEQ ID NO:1.

11. The AMH analogue according to any one of claims 5 to 7, wherein the amino acid substitution is selected from the group consisting of (i) G533, (ii) L535 and (iii) G533+L535.

12. The AMH analogue according to any one of claims 8 to 11, wherein the substitution is selected from the group consisting of (i) L535M and (ii) G533A+L535M.

13. The AMH analogue according to any one of claims 1 to 12, wherein the modification is selected from the group consisting of (i) G533A, (ii) G533S, (iii) G533K, (iv) G533L and (v) G533R.

14. The AMH analogue according to any one of claims 1 to 13, wherein the modification is G533K.

15. The AMH analogue according to any one of claims 1 to 14 wherein the C-terminal domain comprises a sequence selected from the group consisting of:
(SEQ ID NO: 9)
(i)SAGATAADGPCALRELSVDLRAERSVLIPETYQANNCQGVCGWPQSD
RNPRYGNHVVLLLKMQARGAALARPPCCVPTAYAKKLLISLSEERISAHH
VPNMVATECGCR;
(SEQ ID NO: 10)
(ii)SAGATAADGPCALRELSVDLRAERSVLIPETYQANNCQGVCGWPQS
DRNPRYGNHVVLLLKMQARGAALARPPCCVPTAYASKLLISLSEERISAH
HVPNMVATECGCR;
(SEQ ID NO: 11)
(iii)SAGATAADGPCALRELSVDLRAERSVLIPETYQANNCQGVCGWPQ
SDRNPRYGNHVVLLLKMQARGAALARPPCCVPTAYAAKLLISLSEERISA
HHVPNMVATECGCR;
(SEQ ID NO: 12)
(iv)SAGATAADGPCALRELSVDLRAERSVLIPETYQANNCQGVCGWPQS
DRNPRYGNHVVLLLKMQARGAALARPPCCVPTAYAHKLLISLSEERISAH
HVPNMVATECGCR;
(SEQ ID: 13)
(v)SAGATAADGPCALRELSVDLRAERSVLIPETYQANNCQGVCGWPQSD
RNPRYGNHVVLLLKMQARGAALARPPCCVPTAYAGKMLISLSEERISAHH
VPNMVATECGCR;
(SEQ ID NO: 14)
(vi)SAGATAADGPCALRELSVDLRAERSVLIPETYQANNCQGVCGWPQS
DRNPRYGNHVVLLLKMQARGAALARPPCCVPTAYAAKMLISLSEERISAH
HVPNMVATECGCR;
and
(SEQ ID NO: 15)
(vii)SAGATAADGPCALRELSVDLRAERSVLIPETYQANNCQGVCGWPQ
SDRNPRYGNHVVLLLKMQARGAALARPPCCVPTAYAAKMLISLSEERISA
HKVPNMVATECGCR.

16. The AMH analogue according to any one of claims 1 to 15, wherein the C-terminal domain optionally comprises one or more further amino acid residues at the N-terminus.

17. The AMH analogue according claim 16, wherein the one or more further amino acid residues at the N-terminus comprise SVSS (SEQ ID NO:41).

18. The AMH analogue according to any one of claims 1 to 17 further comprising a fusion partner selected from one or more of an Fc protein, a detection tag, a purification tag, or a carrier molecule.

19. An AMH precursor comprising a polypeptide comprising a C-terminal domain sequence, wherein the C-terminal domain comprises at least one amino acid residue modification relative to a native human mature processed AMH polypeptide set forth in SEQ ID NO:5, wherein the modification is present within one or more of amino acid residues 533 to 548 of SEQ ID NO:1.

20. The AMH precursor according to claim 19, wherein the modification is G533A, G533K or G533S.

21. The AMH precursor according to claim 19 or claim 20, wherein the polypeptide comprises the sequence set forth in SEQ ID NO:1 or SEQ ID NO:3, wherein the amino acid corresponding to G533 of SEQ ID NO:1 is modified by substitution to G533A, G533S or G533K.

22. An AMH polynucleotide comprising the sequence set forth in SEQ ID NO:4, wherein the polynucleotide sequence at nucleotides 1603 to 1605 of SEQ ID NO:4 is modified to encode an alanine (A), serine (S) or lysine (K).

23. An AMH polynucleotide comprising the sequence set forth in SEQ ID NO:34, wherein the polynucleotide sequence at nucleotides 1597 to 1599 of SEQ ID NO:33 is modified to encode an alanine (A), serine (S) or lysine (K).

24. An AMH polynucleotide comprising the sequence set forth in SEQ ID NO:35, wherein the polynucleotide sequence at nucleotides 244 to 246 is modified to encode an alanine (A), serine (S) or lysine (K).

25. A polynucleotide encoding the AMH analogue according to any one of claims 1 to 18 or the AMH precursor according to any one of claims 19 to 21.

26. A vector comprising the polynucleotide according to any one of claims 22 to 25.

27. The vector according to claim 26 which is an AAV vector.

28. A host cell comprising the vector according to claim 26 or 27.

29. A composition comprising the AMH analogue according to any one of claims 1 to 18, a polynucleotide according to any one of claims 22 to 25 or the vector according to claim 26 or 27.

30. The composition according to claim 29, wherein the composition is administered in combination with a cell therapeutic, immunotherapeutic, chemotherapeutic or radiotherapeutic agent.

31. A method of preventing a decline in the functional ovarian reserve in a female subject, comprising administering to the subject, the AMH analogue according to any one of claims 1 to 18 or the composition according to claim 29 or 30.

32. A method of contraception in a female subject, comprising administering to the subject the AMH analogue according to any one of claims 1 to 18 or the composition according to claim 29 or 30.

33. A method for ovarian and/or uterine protection in a subject, comprising administering to the subject the AMH analogue according to any one of claims 1 to 18 or the composition according to claim 29 or 30.

34. A method for treating gynaecological cancer in a subject, comprising administering to the subject the AMH analogue according to any one of claims 1 to 18 or the composition according to claim 29 or 30.

35. The method according to any one of claims 31 to 34, wherein the subject is undergoing or about to undergo treatment for cancer or is undergoing treatment or about to undergo treatment for a chronic disease or disorder.

36. The method according to claim 35, wherein the subject has an autoimmune disease and will be treated with, or is currently being treated with, or has been treated with, an immunotherapy, or the subject will be treated with, or is currently being treated with, or has been treated with a cytotoxic drug or cytotoxic agent that causes cell death or cell damage to cells in the uterus or ovary.

37. The method according to any one of claims 31 to 36, wherein the subject is a human.

38. The method according to any one of claims 31 to 36, wherein the subject is a non-human animal selected from cat, dog and horse.

39. Use of an AMH analogue according to any one of claims 1 to 18 in the manufacture of a medicament for preserving ovarian follicle reserve, contraception, uterine protection, or treating a gynaecological cancer.

40. The method according to any one of claims 31 to 38 or use according to claim 39 wherein the AMH analogue is administered as a quaternary complex comprising an N-terminal homodimer and a C-terminal homodimer.

41. A kit for use according to the method of any one of claims 31 to 38 comprising:
(i) an administration device comprising the AMH analogue according to any one of claims 1 to 18; and
(ii) instructions for use in a subject.


SEQUENCE LISTING (41 sequences)

SEQ ID NO: 1 - protein, 560 aa, Homo sapiens
MRDLPLTSLALVLSALGALLGTEALRAEEPAVGTSGLIFREDLDWPPGSPQEPLCLVALGGDSNGSSSPLRVVGALSAYEQAFLGAVQRARWGPRDLATFGVCNTGDRQAALPSLRRLGAWLRDPGGQRLVVLHLEEVTWEPTPSLRFQEPPPGGAGPPELALLVLYPGPGPEVTVTRAGLPGAQSLCPSRDTRYLVLAVDRPAGAWRGSGLALTLQPRGEDSRLSTARLQALLFGDDHRCFTRMTPALLLLPRSEPAPLPAHGQLDTVPFPPPRPSAELEESPPSADPFLETLTRLVRALRVPPARASAPRLALDPDALAGFPQGLVNLSDPAALERLLDGEEPLLLLLRPTAATTGDPAPLHDPTSAPWATALARRVAAELQAAAAELRSLPGLPPATAPLLARLLALCPGGPGGLGDPLRALLLLKALQGLRVEWRGRDPRGPGRAQRSAGATAADGPCALRELSVDLRAERSVLIPETYQANNCQGVCGWPQSDRNPRYGNHVVLLLKMQARGAALARPPCCVPTAYAGKLLISLSEERISAHHVPNMVATECGCR

SEQ ID NO: 2 - protein, 554 aa, Mus musculus
MQGPHLSPLVLLLATMGAVLQPEAVENLATNTRGLIFLEDELWPPSSPPEPLCLVTVRGEGNTSRASLRVVGGLNSYEYAFLEAVQESRWGPQDLATFGVCSTDSQATLPALQRLGAWLGETGEQQLLVLHLAEVIWEPELLLKFQEPPPGGASRWEQALLVLYPGPGPQVTVTGTGLRGTQNLCPTRDTRYLVLTVDFPAGAWSGSGLILTLQPSREGATLSIDQLQAFLFGSDSRCFTRMTPTLVVLPPAEPSPQPAHGQLDTMPFPQPGLSLEPEALPHSADPFLETLTRLVRALRGPLTQASNTQLALDPGALASFPQGLVNLSDPAALGRLLDWEEPLLLLLSPAAATEREPMPLHGPASAPWAAGLQRRVAVELQAAASELRDLPGLPPTAPPLLARLLALCPNDSRSSGDPLRALLLLKALQGLRAEWHGREGRGRTGRSAGTGTDGPCALRELSVDLRAERSVLIPETYQANNCQGACAWPQSDRNPRYGNHVVLLLKMQARGAALGRLPCCVPTAYAGKLLISLSEERISAHHVPNMVATECGCR

SEQ ID NO: 3 - protein, 562 aa, Artificial Sequence
  modified AMH precursor
MKWVTFLLLLFISGSAFSHHHHHHPAVGTSGLIFREDLDWPPGSPQEPLCLVALGGDSNGSSSPLRVVGALSAYEQAFLGAVQRARWGPRDLATFGVCNTGDRQAALPSLRRLGAWLQDPGGQRLVVLHLEEVTWEPTPSLRFQEPPPGGAGPPELALLVLYPGPGPEVTVTRAGLPGAQSLCPSRDTRYLVLAVDRPAGAWRGSGLALTLQPRGEDSRLSTARLQALLFGDDHRCFTRMTPALLLLPRSEPAPLPAHGQLDTVPFPPPRPSAELEESPPSADPFLETLTRLVRALRVPPARASAPRLALDPDALAGFPQGLVNLSDPAALERLLDGEEPLLLLLRPTAATTGDPAPLHDPTSAPWATALARRVAAELQAAAAELRSLPGLPPATAPLLARLLALCPGGPGGLGDPLRALLLLKALQGLRVEWRGRDPRGPGISSRKKRSVSSSAGATAADGPCALRELSVDLRAERSVLIPETYQANNCQGVCGWPQSDRNPRYGNHVVLLLKMQARGAALARPPCCVPTAYAGKLLISLSEERISAHHVPNMVATECGCR

SEQ ID NO: 4 - DNA, 1689 bp, Artificial Sequence
  modified AMH precursor
atgaagtgggtaacctttctcctcctcctcttcatctccggttctgccttttcccatcatcatcatcatcatccagctgtgggcaccagtggcctcatcttccgagaagacttggactggcctccaggcagcccacaagagcctctgtgcctggtggcactgggcggggacagcaatggcagcagctcccccctgcgggtggtgggggctctaagcgcctatgagcaggccttcctgggggctgtgcagagggcccgctggggcccccgagacctggccaccttcggggtctgcaacaccggtgacaggcaggctgccttgccctctctacggcggctgggggcctggctgcaggaccctggggggcagcgcctggtggtcctacacctggaggaagtgacctgggagccaacaccctcgctgaggttccaggagcccccgcctggaggagctggccccccagagctggcgctgctggtgctgtaccctgggcctggccctgaggtcactgtgacgagggctgggctgccaggtgcccagagcctctgcccctcccgagacacccgctacctggtgttagcggtggaccgccctgcgggggcctggcgcggctccgggctggccttgaccctgcagccccgcggagaggactcccggctgagtaccgcccggctgcaggcactgctgttcggcgacgaccaccgctgcttcacacggatgaccccggccctgctcctgctgccgcggtccgagcccgcgccgctgcctgcgcacggccagctggacaccgtgcccttcccgccgcccaggccatccgcggaactggaggagtcgccacccagcgcagaccccttcctggagacgctcacgcgcctggtgcgggcgctgcgggtccccccggcccgggcctccgcgccgcgcctggccctggatccggacgcgctggccggcttcccgcagggcctagtcaacctgtcggaccccgcggcgctggagcgcctactcgacggcgaggagccgctgctgctgctgctgaggcccactgcggccaccaccggggatcctgcgcccctgcacgaccccacgtcggcgccgtgggccacggccctggcgcgccgcgtggctgctgaactgcaagcggcggctgccgagctgcgaagcctcccgggtctgcctccggccacagccccgctgctggcgcgcctgctcgcgctctgtccaggaggccccggcggcctcggcgatcccctgcgagcgctgctgctcctgaaggcgctgcagggcctgcgcgtggagtggcgcgggcgggatccgcgcgggccgggtatctcatcgagaaagaaacgctcagtctcatcaagcgcgggggccaccgccgccgacgggccgtgcgcgctgcgcgagctcagcgtagacctccgcgccgagcgctccgtactcatccccgagacctaccaggccaacaattgccagggcgtgtgcggctggcctcagtccgaccgcaacccgcgctacggcaaccacgtggtgctgctgctgaagatgcaggcccgtggggccgccctggcgcgcccaccctgctgcgtgcccaccgcctacgcgggcaagctgctcatcagcctgtcggaggagcgcatcagcgcgcaccacgtgcccaacatggtggccaccgagtgtggctgccggtaa

SEQ ID NO: 5 - protein, 109 aa, Artificial Sequence
  modified mature AMH sequence
SAGATAADGPCALRELSVDLRAERSVLIPETYQANNCQGVCGWPQSDRNPRYGNHVVLLLKMQARGAALARPPCCVPTAYAGKLLISLSEERISAHHVPNMVATECGCR

SEQ ID NO: 6 - protein, 11 aa, Artificial Sequence
  processing site
ISSRKKRSVSS

SEQ ID NO: 7 - protein, 25 aa, Artificial Sequence
  signal sequence
MRDLPLTSLALVLSALGALLGTEAL

SEQ ID NO: 8 - protein, 18 aa, Artificial Sequence
  signal sequence
MKWVTFLLLLFISGSAFS

SEQ ID NO: 9 - protein, 109 aa, Artificial Sequence
  AMH analogue
SAGATAADGPCALRELSVDLRAERSVLIPETYQANNCQGVCGWPQSDRNPRYGNHVVLLLKMQARGAALARPPCCVPTAYAKKLLISLSEERISAHHVPNMVATECGCR

SEQ ID NO: 10 - protein, 109 aa, Artificial Sequence
  AMH analogue
SAGATAADGPCALRELSVDLRAERSVLIPETYQANNCQGVCGWPQSDRNPRYGNHVVLLLKMQARGAALARPPCCVPTAYASKLLISLSEERISAHHVPNMVATECGCR

SEQ ID NO: 11 - protein, 109 aa, Artificial Sequence
  AMH analogue
SAGATAADGPCALRELSVDLRAERSVLIPETYQANNCQGVCGWPQSDRNPRYGNHVVLLLKMQARGAALARPPCCVPTAYAAKLLISLSEERISAHHVPNMVATECGCR

SEQ ID NO: 12 - protein, 109 aa, Artificial Sequence
  AMH analogue
SAGATAADGPCALRELSVDLRAERSVLIPETYQANNCQGVCGWPQSDRNPRYGNHVVLLLKMQARGAALARPPCCVPTAYAHKLLISLSEERISAHHVPNMVATECGCR

SEQ ID NO: 13 - protein, 109 aa, Artificial Sequence
  AMH analogue
SAGATAADGPCALRELSVDLRAERSVLIPETYQANNCQGVCGWPQSDRNPRYGNHVVLLLKMQARGAALARPPCCVPTAYAGKMLISLSEERISAHHVPNMVATECGCR

SEQ ID NO: 14 - protein, 109 aa, Artificial Sequence
  AMH analogue
SAGATAADGPCALRELSVDLRAERSVLIPETYQANNCQGVCGWPQSDRNPRYGNHVVLLLKMQARGAALARPPCCVPTAYAAKMLISLSEERISAHHVPNMVATECGCR

SEQ ID NO: 15 - protein, 109 aa, Artificial Sequence
  AMH analogue
SAGATAADGPCALRELSVDLRAERSVLIPETYQANNCQGVCGWPQSDRNPRYGNHVVLLLKMQARGAALARPPCCVPTAYAAKMLISLSEERISAHKVPNMVATECGCR

SEQ ID NO: 16 - protein, 21 aa, Artificial Sequence
  signal sequence
MKVSVAALSCLMLVTALGSQA

SEQ ID NO: 17 - protein, 17 aa, Artificial Sequence
  signal sequence
MLQNSAVLLLLVISASA

SEQ ID NO: 18 - protein, 19 aa, Artificial Sequence
  signal sequence
MLLQAFLFLLAGFAAKISA

SEQ ID NO: 19 - protein, 24 aa, Artificial Sequence
  signal sequence
MKWVSFISLLFLFSSAYSRSLEKR

SEQ ID NO: 20 - protein, 24 aa, Artificial Sequence
  signal sequence
MKWVSFISLLFLFSSAYSRSLDKR

SEQ ID NO: 21 - protein, 21 aa, Artificial Sequence
  signal sequence
MNIFYIFLFLLSFVQGSLDKR

SEQ ID NO: 22 - protein, 19 aa, Artificial Sequence
  signal sequence
MGWSCIILFLVATATGVHS

SEQ ID NO: 23 - protein, 29 aa, Artificial Sequence
  signal sequence
MERAAPSRRVPLPLLLLGGLALLAAGVDA

SEQ ID NO: 24 - protein, 22 aa, Artificial Sequence
  signal sequence
MMKTLLLFVGLLLTWESGQVLG

SEQ ID NO: 25 - protein, 21 aa, Artificial Sequence
  signal sequence
MLPLCLVAALLLAAGPGPSLG

SEQ ID NO: 26 - protein, 18 aa, Artificial Sequence
  Signal sequence
MKWVTFISLLFLFSSAYS

SEQ ID NO: 27 - DNA, 1665 bp, Mus musculus
atgcaggggccacacctctctccactggtactgctgctagcgactatgggggctgtgttacagcctgaggcggttgaaaacctggccaccaataccaggggcctcatcttccttgaagatgagctctggccccccagcagcccccctgaacctttgtgcctggtgacagtgagaggagaggggaacacaagcagagcttccctgagggtggtggggggtctgaacagctatgagtatgccttcctggaggctgtccaggagtctcgctggggaccccaagacctggccaccttcggagtctgcagcactgactcccaggctaccctgcctgccctgcagcgccttggggcctggctaggggagactggagaacagcagttgctagtcctacatctggctgaagtgatatgggagcccgagctcttgctgaagttccaagagcctccacctgggggagccagccgctgggagcaggccctgttagtgctataccctggaccaggcccccaggtcacagtcacagggactggactgcggggcacacagaacctctgccctactcgggacacccgctatttggtgctaaccgtggacttcccagcgggggcctggagcggctcgggcctcatcttaacccttcaaccaagcagagaaggtgccaccctgagcatcgatcagctgcaagcctttctatttggctctgattcccgctgtttcacgcggatgactcccaccctggtggtgctgccacccgccgagccgtcaccgcagccagcacacggccagctggacaccatgcctttcccgcagcctggactgtccctggagcctgaggccctgccacacagcgccgaccccttcctagagaccctcactcgcttggttcgtgctctgcggggacctctgacccaggcttcgaacacgcaactggccctggaccctggtgcgctggccagcttcccacagggcctggtcaacctgtcagaccccgcagcactgggacgcctgctcgactgggaggaacccctattactgctgctgtcacccgctgcggccacggagagggaacctatgccgctgcacggccccgcttctgctccctgggcagcgggcctgcaacgcagggtggcagtggagctgcaggcggcagcctcagagctgcgggacctcccgggtctgccacccacagctcccccgctgctggcgcgcctgctagcgctgtgtcccaacgactcccgcagctccggggacccgctgcgcgcgctgctgctgctaaaggcgctgcagggcttacgtgccgagtggcatgggcgggaagggcgtgggagaacggggcgcagcgcggggacagggacagacgggccttgcgcgctgcgcgagctgagtgtagatctgcgcgcggagcgttcagtgctcatcccggagacctaccaagccaacaactgccaaggcgcctgcgcgtggccgcagtctgaccgtaatccgcgctacgggaaccacgtggtgctgctgctaaaaatgcaggctcgcggggctgccctgggccgcctgccctgctgcgtgcccactgcctacgcgggcaagctgctcatcagcctgtccgaggagcgcatcagcgcgcaccacgtgcccaacatggtagccaccgagtgcggctgccggtga

SEQ ID NO: 28 - DNA, 1722 bp, Equus caballus
atgcgggctccgtctctctcgtggctggcgcttgtgctgtcagctgtgggggctctgctgagggccgggacccccggagaagaggtctccagcaccccagctttgcctggggagccagccacaggcaccgggggcctcatcttccaccaggactgggactggccgccaggcagcccccaagaacccctggggtgcttggtgacgctggacgagggtggcaaccagagcagcgccccgctgcgggtggcgggggccctgagcggctacgagcaggccttcctggaggccgtgcagcgcacccactggagcccccgcgacctgcccaccttcggggtctgctcccctgccgaccggcaggcggccgtaccatctctgcagcgcctgcaggcctggctgggggcaccctgggggcagcggctggtggtcctgcacctggaggaagtgatgtgggagccgacgccctcgctgaggttccaggagcccccatctggaggagccaaccccctggagccagcgctgctggtgctgtacgccgggcccggccctgaggtcactgtcacgggggccgggctgccaggggcccagagcctctgcccgtccccggacacccgctacctggcgctggccgtcgaccacccagcacgggcctggcgccaccctgggctcatcctgaccctgcagcctcgcggagacggtgcccccctgagcacggcccagctgcagacgctgctgttcggcgccgacccccgctgcttcacgcggatgacccccgccctgttcctgctgcagcggccggggcccgcgcccatgcccgcgcacggccgactcgacacggtgcccttcccgccagccaggccgtccccggagcccgaggagccgcggcccagcgccgaccccttcctggagacgctcacgcgcctggtgcgcgcgctgcgggggcccccgactccggcctcgccgccacgcctggccctggacccgggcgcgctggccagcttcccgcagggcctggtcaacctgtcggaccccgcggcgctggagcgcctgctcgacggcgaggagccgctgctgctgctgctgccgcccaccgcggccgctgccggggaccccgcgccattgccggaccccgcgtcggcgccctgggccgcgggcctggcgcgccgcgtggccgccgagctgcaggcggcggcggccgagctgcgcagcctcccggggctgccccccgccgcagagccgctgctggcgcgcctgctcgcactgtgcccgggggacgccgaggaccagggcggcccgggcggcccgctgcgcgcgctgctgctgctgaaggcgctgcaaggcctgcgcgccgagtggcgcgggcgcgagcggagcgggcccgggcgggcgcagcgcaacgcgggggccggggccgccgacgggccgtgcgcgctgcgcgagctgcgcgtggacttgcgcgccgagcgctccgtgctcatccccgagacgtaccaggccaacaactgccagggcgcgtgcggctggccgcagtcggaccgcaacccgcgctacggcaaccacgtggtgctgctgctaaagatgcaggcccgcggcgccgccctggcgcgcgcgccctgctgcgtgcccactgcctacgcgggcaagctgctcatcagcctgtccgaggagcgcatcagcgcgcaccacgtgcccaacatggtggccaccgagtgcggctgccggtga

SEQ ID NO: 29 - DNA, 1719 bp, Canis lupus familiaris
atgggggctctggcgctctggccgctggccctggcgctgtcggggatggggcccctgctgggagccgaggcccccggaggtgaggtctcgggcaccccagcctcgcccggagagccggccacaggcactgggggtctcctcttccagccagactgggactggccgcctagcgccccacaagaccccctgtgcctggtgaccctggacaagggaggcaacgggagcagccccccgcttcgggtggcgggggccctccgaggctacgagcacaccttcctcgaggccgtgcggcgagcccgctggggtccccacgacctggccaccttcggggcctgcgccgccagcgacggccggaccacccagctctccctgcggcagctgcaagcctggctgggggcgcccggggggcggcggctggtggtgctgcacctggaggaagtgacatgggagccagcgctgtcgctgaagttccaggagcccccacctggaggagccagtcccctagagctggcgctgctggtgctctaccctgggccaggccctgaggtcgctgtcaccggggctggactgccaggcacccagaacctttgccggtcccggaacacgcgctacctggtgctggccctggaccacccggtgggggcctggcacagccctagggtcaccctgaccgtgcacgcccgaggagacggtgccccgctgagcaccccccagctgcaggagctgctgttcgggcccgatgcccgctgcttcacgcggatgacgcccgcgctgctcgtgctgcggctgcccgggcccacggcggtgccggcgcgcggcctgctggacctcgtgcccttcccgccgcccaggccctcccgggagcccgcggagccccctcccagcgcagaccccttcctggagacgctcacgcgcctggtgcgcgccctgcgcgggccgccgacccccgcctcgccgccgcgcctggccctggaccccggcgcgctggcgggcttcccgcagggcctgctcaacctgtcggaccccgcgacacaggagcgcctgctgggcggcgaggagcccctgctgctgctgctgccaccccccacggccgcggccgggccccccgcgccgccgccccgccccgcgtccgcgccctgggccgcgggcctcgccctgcgcgtggccgccgagctgcgggccgcggccgccgagctccgcgggctcccggggctgccccccgccgccgcgccgctgctggagcgcctgctcgccctctgccccgggggctcggggggctcggggggctcgggggacccgctgcgcgcgctgctgctgctcaaggcgttgcagggcctgcgcgccgagtggcgcgggcgggagcggggcgggcccccccgggcgcagcgcagcgcgggggcgggggcggccgacgggccctgcgcgctgcgcgagctgagcgtggacctgcgcgccgagcgctccgtgctcatccccgagacctaccaggccaacaactgccagggcgcgtgcgggtggccgcagtccgaccgcaacccgcgctacggcaaccacgtggtgctgctgctcaagatgcaggcccgcggcgccgcgctggctcgcccgccctgctgcgtgcccacggcctacggcggcaagctgctcatcagcctgtcggaggagcgcatcagcgcgcaccacgtgcccaacatggtggccaccgagtgcggctgccggtga

SEQ ID NO: 30 - DNA, 1767 bp, Felis catus
atgcctggtctgctctctccgccggccctggtgctgtcggtgatgggggctctgctgatggccggggaccctggggaagaggtctccagcaccccggccctgcctggagggccagccacaggcaccgggggtctcatcttccacccggattgggactggcagcccccgggcagtccccaagaccccctgtgcctggtgacgctggacaggggtggtaacgggagcggctccccgcttcgggtggtgggggcgctgagaggctacgagcacgccttcctcgaggctgtgcggcgggcccgctggggtccccacggcctggccaccttcggagtttgcacccccagggacaggcaggccgccccgttctctctgcggcagctgcaggcgtggctgggggagcccggggggcggcggctggtggtgctgcacctggaggaagtgacatgggagccgacaccctcactgaagttccaggagcccccgcctggaggggccggtcccctagagctggcgatgctggtgctgtaccctggccctggccccgaggtcacggtcacaggggctgggctgccaggcacccagagcctctgtcagtcccgggacacccgctacctggtgctggcggtggaccacccagaaggggcctggcgcagccccgggctcaccctgaccctgcaaccccgcagagacggtgcgcccctgagcaccgcccagctgcaggagctgctgttcggccccgacccccgctgcttcacacgcatgaccccggcgctgctcctgctgccggggcccgcgcctgcaccgctgcccgcgcgtggcctgctggaccaagtgcctctcccgccgcccaggccctcccaggagcaggcgcccgaggagccacggtccagcgccgaccccttcctggagacgctcacgcgcctggtgcgcgcgctgcgggggcccccggcccaggcctcgccggcgcgcctggccctggaccccggcgcgctggccggcttcccgcagggcctggtcaacctgtcggaccccgcggcgcaggagcgcctgctcaacggcggcgacgagccgctgctgctgcttctgctgccacccgccacgcccaccgccgccgccgccgccgccggggaccccgcgccgccgcgcggcccggcgtccgcgccctgggccgccggcctagcgcgtcgcgtggccgccgagctgcaggccgcggccgccgagctgcgagggctcccggggctgccgccggccgccacgccgctgctggcgcgcctgctcgcgctgtgccccggggattcgggggactcgggggacccgggggccccccccggcggcccgggcggcccgctgcgcgcgctgctgctgctcaaggcgctgcagggtctgcgcgcggagtggcgcgggcgcgagcaggccggaccggcgcgggcacagcgcagcgcgggggccggggcggccgacgggccgtgcgcgctgcgcgagctgagcgtggacctgcgcgccgagcgctccgtgctcatcccggagacgtaccaggccaacaactgccagggcgcgtgcggctggccgcagtccgaccgcaacccgcgctacggcaaccacgtggtgctgctgctcaagatgcaggcccgcggcgccgccctggcgcgcccgccctgctgcgtgcccacggcctacgcgggcaagctcctcatcagcctgtcggaggagcgcatcagcgcgcaccacgtgcccaacatggtggccaccgagtgcggctgccggtga

SEQ ID NO: 31 - protein, 573 aa, Equus caballus
MRAPSLSWLALVLSAVGALLRAGTPGEEVSSTPALPGEPATGTGGLIFHQDWDWPPGSPQEPLGCLVTLDEGGNQSSAPLRVAGALSGYEQAFLEAVQRTHWSPRDLPTFGVCSPADRQAAVPSLQRLQAWLGAPWGQRLVVLHLEEVMWEPTPSLRFQEPPSGGANPLEPALLVLYAGPGPEVTVTGAGLPGAQSLCPSPDTRYLALAVDHPARAWRHPGLILTLQPRGDGAPLSTAQLQTLLFGADPRCFTRMTPALFLLQRPGPAPMPAHGRLDTVPFPPARPSPEPEEPRPSADPFLETLTRLVRALRGPPTPASPPRLALDPGALASFPQGLVNLSDPAALERLLDGEEPLLLLLPPTAAAAGDPAPLPDPASAPWAAGLARRVAAELQAAAAELRSLPGLPPAAEPLLARLLALCPGDAEDQGGPGGPLRALLLLKALQGLRAEWRGRERSGPGRAQRNAGAGAADGPCALRELRVDLRAERSVLIPETYQANNCQGACGWPQSDRNPRYGNHVVLLLKMQARGAALARAPCCVPTAYAGKLLISLSEERISAHHVPNMVATECGCR

SEQ ID NO: 32 - protein, 572 aa, Canis lupus familiaris
MGALALWPLALALSGMGPLLGAEAPGGEVSGTPASPGEPATGTGGLLFQPDWDWPPSAPQDPLCLVTLDKGGNGSSPPLRVAGALRGYEHTFLEAVRRARWGPHDLATFGACAASDGRTTQLSLRQLQAWLGAPGGRRLVVLHLEEVTWEPALSLKFQEPPPGGASPLELALLVLYPGPGPEVAVTGAGLPGTQNLCRSRNTRYLVLALDHPVGAWHSPRVTLTVHARGDGAPLSTPQLQELLFGPDARCFTRMTPALLVLRLPGPTAVPARGLLDLVPFPPPRPSREPAEPPPSADPFLETLTRLVRALRGPPTPASPPRLALDPGALAGFPQGLLNLSDPATQERLLGGEEPLLLLLPPPTAAAGPPAPPPRPASAPWAAGLALRVAAELRAAAAELRGLPGLPPAAAPLLERLLALCPGGSGGSGGSGDPLRALLLLKALQGLRAEWRGRERGGPPRAQRSAGAGAADGPCALRELSVDLRAERSVLIPETYQANNCQGACGWPQSDRNPRYGNHVVLLLKMQARGAALARPPCCVPTAYGGKLLISLSEERISAHHVPNMVATECGCR

SEQ ID NO: 33 - protein, 588 aa, Felis catus
MPGLLSPPALVLSVMGALLMAGDPGEEVSSTPALPGGPATGTGGLIFHPDWDWQPPGSPQDPLCLVTLDRGGNGSGSPLRVVGALRGYEHAFLEAVRRARWGPHGLATFGVCTPRDRQAAPFSLRQLQAWLGEPGGRRLVVLHLEEVTWEPTPSLKFQEPPPGGAGPLELAMLVLYPGPGPEVTVTGAGLPGTQSLCQSRDTRYLVLAVDHPEGAWRSPGLTLTLQPRRDGAPLSTAQLQELLFGPDPRCFTRMTPALLLLPGPAPAPLPARGLLDQVPLPPPRPSQEQAPEEPRSSADPFLETLTRLVRALRGPPAQASPARLALDPGALAGFPQGLVNLSDPAAQERLLNGGDEPLLLLLLPPATPTAAAAAAGDPAPPRGPASAPWAAGLARRVAAELQAAAAELRGLPGLPPAATPLLARLLALCPGDSGDSGDPGAPPGGPGGPLRALLLLKALQGLRAEWRGREQAGPARAQRSAGAGAADGPCALRELSVDLRAERSVLIPETYQANNCQGACGWPQSDRNPRYGNHVVLLLKMQARGAALARPPCCVPTAYAGKLLISLSEERISAHHVPNMVATECGCR

SEQ ID NO: 34 - protein, 1683 aa, Homo sapiens
ATGCGGGACCTGCCTCTCACCAGCCTGGCCCTAGTGCTGTCTGCCCTGGGGGCTCTGCTGGGGACTGAGGCCCTCAGAGCAGAGGAGCCAGCTGTGGGCACCAGTGGCCTCATCTTCCGAGAAGACTTGGACTGGCCTCCAGGCAGCCCACAAGAGCCTCTGTGCCTGGTGGCACTGGGCGGGGACAGCAATGGCAGCAGCTCCCCCCTGCGGGTGGTGGGGGCTCTAAGCGCCTATGAGCAGGCCTTCCTGGGGGCCGTGCAGAGGGCCCGCTGGGGCCCCCGAGACCTGGCCACCTTCGGGGTCTGCAACACCGGTGACAGGCAGGCTGCCTTGCCCTCTCTACGGCGGCTGGGGGCCTGGCTGCGGGACCCTGGGGGGCAGCGCCTGGTGGTCCTACACCTGGAGGAAGTGACCTGGGAGCCAACACCCTCGCTGAGGTTCCAGGAGCCCCCGCCTGGAGGAGCTGGCCCCCCAGAGCTGGCGCTGCTGGTGCTGTACCCTGGGCCTGGCCCTGAGGTCACTGTGACGAGGGCTGGGCTGCCGGGTGCCCAGAGCCTCTGCCCCTCCCGAGACACCCGCTACCTGGTGTTAGCGGTGGACCGCCCTGCGGGGGCCTGGCGCGGCTCCGGGCTGGCCTTGACCCTGCAGCCCCGCGGAGAGGACTCCCGGCTGAGTACCGCCCGGCTGCAGGCACTGCTGTTCGGCGACGACCACCGCTGCTTCACACGGATGACCCCGGCCCTGCTCCTGCTGCCGCGGTCCGAGCCCGCGCCGCTGCCTGCGCACGGCCAGCTGGACACCGTGCCCTTCCCGCCGCCCAGGCCATCCGCGGAACTCGAGGAGTCGCCACCCAGCGCAGACCCCTTCCTGGAGACGCTCACGCGCCTGGTGCGGGCGCTGCGGGTCCCCCCGGCCCGGGCCTCCGCGCCGCGCCTGGCCCTGGATCCGGACGCGCTGGCCGGCTTCCCGCAGGGCCTAGTCAACCTGTCGGACCCCGCGGCGCTGGAGCGCCTACTCGACGGCGAGGAGCCGCTGCTGCTGCTGCTGAGGCCCACTGCGGCCACCACCGGGGATCCTGCGCCCCTGCACGACCCCACGTCGGCGCCGTGGGCCACGGCCCTGGCGCGCCGCGTGGCTGCTGAACTGCAAGCGGCGGCTGCCGAGCTGCGAAGCCTCCCGGGTCTGCCTCCGGCCACAGCCCCGCTGCTGGCGCGCCTGCTCGCGCTCTGCCCAGGTGGCCCCGGCGGCCTCGGCGATCCCCTGCGAGCGCTGCTGCTCCTGAAGGCGCTGCAGGGCCTGCGCGTGGAGTGGCGCGGGCGGGATCCGCGCGGGCCGGGTCGGGCACAGCGCAGCGCGGGGGCCACCGCCGCCGACGGGCCGTGCGCGCTGCGCGAGCTCAGCGTAGACCTCCGCGCCGAGCGCTCCGTACTCATCCCCGAGACCTACCAGGCCAACAATTGCCAGGGCGTGTGCGGCTGGCCTCAGTCCGACCGCAACCCGCGCTACGGCAACCACGTGGTGCTGCTGCTGAAGATGCAGGTCCGTGGGGCCGCCCTGGCGCGCCCACCCTGCTGCGTGCCCACCGCCTACGCGGGCAAGCTGCTCATCAGCCTGTCGGAGGAGCGCATCAGCGCGCACCACGTGCCCAACATGGTGGCCACCGAGTGTGGCTGCCGGTGA

SEQ ID NO: 35 - DNA, 330 bp, Artificial Sequence
  mature processed AMH
agcgcgggggccaccgccgccgacgggccgtgcgcgctgcgcgagctcagcgtagacctccgcgccgagcgctccgtactcatccccgagacctaccaggccaacaattgccagggcgtgtgcggctggcctcagtccgaccgcaacccgcgctacggcaaccacgtggtgctgctgctgaagatgcaggtccgtggggccgccctggcgcgcccaccctgctgcgtgcccaccgcctacgcgggcaagctgctcatcagcctgtcggaggagcgcatcagcgcgcaccacgtgcccaacatggtggccaccgagtgtggctgccggtga

SEQ ID NO: 36 - protein, 113 aa, Artificial Sequence
  Mature processed AMH
SVSSSAGATAADGPCALRELSVDLRAERSVLIPETYQANNCQGVCGWPQSDRNPRYGNHVVLLLKMQARGAALARPPCCVPTAYAGKLLISLSEERISAHHVPNMVATECGCR

SEQ ID NO: 37 - protein, 11 aa, Artificial Sequence
  protease cleavage site
  X (1)..(1): X is absent or isoleucine
  X (2)..(2): X is absent or serine
  X (3)..(3): X is absent or serine
  X (8)..(8): X is absent or serine
  X (9)..(9): X is absent or isoleucine
  X (10)..(10): X is absent or serine
  X (11)..(11): X is absent or serine
XXXRKKRXXXX

SEQ ID NO: 38 - protein, 4 aa, Artificial Sequence
  protease cleavage site
RKKR

SEQ ID NO: 39 - protein, 7 aa, Artificial Sequence
  protease cleavage site
ISSRKKR

SEQ ID NO: 40 - protein, 8 aa, Artificial Sequence
  protease cleavage site
RKKRSVSS

SEQ ID NO: 41 - protein, 4 aa, Artificial Sequence
  N-terminal extension
SVSS